(19) United States
(12) Patent Application Publication
(10) Pub. No.: US 2021/0338626 A1
(43) Pub. Date: Nov. 4, 2021

(54) METHODS FOR TREATING OR PREVENTING A VIRAL INFECTION OR INHIBITING VIRAL REPLICATION

(71) Applicant: Dalcor Pharma UK Ltd., Leatherhead, Zug Branch, Zug (CH)

(72) Inventor: Eric NIESOR, Zug (CH)

(21) Appl. No.: 17/237,777

(22) Filed: Apr. 22, 2021

Related U.S. Application Data
(60) Provisional application No. 63/055,879, filed on Jul. 23, 2020.
(60) Provisional application No. 63/016,922, filed on Apr. 28, 2020.

(51) Int. Cl. A61K 31/265; A61K 45/06; A61P 31/14

(52) U.S. Cl. CPC A61K 31/265; A61P 31/14; A61K 45/06

(57) ABSTRACT

Provided herein are compositions and methods for the treatment or prevention of viral infections, including coronavirus disease (COVID-19), using dalcetrapib, an analog of dalcetrapib, or a pharmaceutically acceptable salt, hydrate or solvate thereof.

### CROSS REFERENCE TO RELATED APPLICATIONS

This application claims the benefit of U.S. Provisional Application No. 63/055,879, filed Jul. 23, 2020, and U.S. Provisional Application No. 63/016,922, filed Apr. 28, 2020, the disclosure of each of which is incorporated by reference herein in its entirety.

### FIELD OF THE INVENTION

The present application is generally directed to the treatment or prevention of a viral infection, such as Coronavirus Disease 2019 (COVID-19), or for inhibiting replication of a virus.

### BACKGROUND

Severe Acute Respiratory Syndrome Coronavirus 2 (SARS-CoV-2) is a virus that causes a respiratory disease called Coronavirus Disease 2019 or COVID-19. COVID-19 is a respiratory disease that was first observed in Wuhan, China in late 2019. Symptoms of COVID-19 vary in each individual. Common symptoms include fever, dry cough, fatigue, loss of appetite, loss of smell, gastrointestinal symptoms, or body ache. In some people, COVID-19 causes more severe symptoms like high fever, severe cough, and shortness of breath, which often indicates pneumonia. Serious cases of COVID-19 may require hospitalization, and may be lethal. Patients over the age of 65 are more likely to die from COVID-19 than younger patients. In early 2020, the World Health Organization (WHO) declared the pandemic of COVID-19 a Public Health Emergency of International Concern.

In some individuals, SARS-CoV-2 infection causes COVID-19. Other individuals infected with SARS-CoV-2 remain asymptomatic. The SARS-CoV-2 genome is about 30,000 nucleotides in size, and has at least 13 open reading frames. SARS-CoV-2 shares about 79.6% genomic sequence identity and the same cellular receptor (angiotensin-converting enzyme-2, ACE-2) with SARS-CoV. Various strains of SARS-CoV-2 have been identified, although sequence homology among all viral strains is generally high (i.e., greater than about 99% at the nucleotide and amino acid level.) SARS-CoV-2 can be an “S-strain” or an “L-strain” of the virus. These two strains are differentiated by nucleotide substitutions at positions 8,782 and 28,144 of the genome. The strains are labeled S- or L- based on the amino acid encoded at amino acid 84 of open reading frame 8 (ORFS).

The SARS-CoV-2 genome encodes several proteins, including the RNA-dependent RNA polymerase, main 3CL protease and papain-like protease which activities represent rate-limiting steps in viral replication. Upon entrance in cells, the viral genome is released as a single-stranded positive RNA and translated into a large viral polyprotein using the host's machinery. This polyprotein is then cleaved at 11 different sites and the recognition sequence at most locations was found to be Leu-GlN(Ser/Ala/Gly). This proteolytic cleavage (indicated by 1) occurs after the amino acid Gln and generates the main 3CL protease and papain-like protease. The catalytic site of these cysteine proteases contains the Cys145-His41 diad and classic Cys112-His273-Asp287 triad for the former and latter, respectively.

There is an urgent need in the art for compositions and methods useful for treating and/or preventing COVID-19.

### BRIEF SUMMARY OF THE INVENTION

Provided herein are compositions and methods useful for treating or preventing a viral infection or for inhibiting replication of a virus in a cell. In particular, provided herein are compositions useful for treating or preventing a viral infection or for inhibiting replication of a virus, comprising an effective amount of an antiviral compound provided herein and a pharmaceutically effective carrier or vehicle.

Also provided herein are methods for treating or preventing a viral infection in a subject in need thereof, comprising administering to the subject an effective amount of an antiviral compound provided herein.

Further provided herein are methods for inhibiting the replication of a virus in a cell, comprising contacting the cell with an effective amount of an antiviral compound provided herein.

Still further provided herein are methods for reducing the risk of acquiring a viral infection in a subject in need thereof, the methods comprising administering to the subject an effective amount of an antiviral compound provided herein.

Still further provided herein are methods for reducing the risk of transmission of a viral infection from a first subject to a second subject, the method comprising administering to the first subject an effective amount of an antiviral compound provided herein.

These and other embodiments of the invention will be described in further detail below.

### BRIEF DESCRIPTION OF THE DRAWINGS

FIGS. 1A-1D show docking of dalcetrapib thiol (Dal-thiol) and dalcetrapib disulfide (Dal-disulfide) in the M^{Pro} of SARS-COV-2. FIG. 1A shows docking of Dal-thiol to the M^{pro} (PDB 6W63) using the SCAR protocol which resulted in a distance of 2.1 Å between the thiol of Dal-thiol and Cys145. The Dal-thiol (carbon in cyan) and the surrounding residues (carbon in green) are shown in stick mode. FIG. 1B shows a surface representation of the image of FIG. 1A with the S1′, S1, S2, S4 sites indicated. FIG. 1C shows docking of Dal-disulfide to the M^{pro} (PDB 6LU7). The carbon atoms of Dal-disulfide and the surrounding residues are shown in light blue and yellow, respectively. FIG. 1D shows a surface representation of the image of FIG. 1C with the S1′, S1, S2, S4 sites indicated. Hydrogen bonds in FIGS. 1A and 1C are shown in black dash lines.

FIG. 2 shows inhibition of the SARS-CoV-2 main 3CL protease enzymatic activity by dalcetrapib.

FIG. 3 shows the effect of dalcetrapib on SARS-CoV-2.

FIG. 4 shows inhibition of the SARS-CoV-2 main 3CL protease enzymatic activity by dalcetrapib, in an in vitro assay.

FIG. 5 is a schematic showing the experimental protocol used to test reversibility of the inhibitory activity of dalcetrapib, as described in Example 6.

FIG. 6 shows inhibition of the proteolytic activity of the SARS-CoV-2 main 3CL protease by dalcetrapib at high (200 uM) concentration and low (˜0.2 μM) concentration.

### DETAILED DESCRIPTION OF THE INVENTION

Unless otherwise defined, all technical and scientific terms used herein have the same meaning as commonly understood by one of skill in the art to which this invention belongs. The terminology used in the detailed description herein is for the purpose of describing particular embodiments only and is not intended to be limiting.

All publications, patent applications, patents, CAS numbers, GenBank or other accession numbers, and other references mentioned herein are incorporated by reference in their entirety for all purposes.

### Definitions

The following terms are used in the description herein and the appended claims.

The singular forms “a,” “an” and “the” are intended to include the plural forms as well, unless the context clearly indicates otherwise.

The term “about” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified value.

As used herein, an “effective amount” of is the amount of an antiviral compound provided herein that is effective to treat or prevent a viral infection in a subject or to inhibit replication of a virus in a cell.

As used herein, the term “COVID-19” refers to Coronavirus Disease 2019.

As used herein, the term “SARS-CoV-2” refers to Severe Acute Respiratory Syndrome Coronavirus 2. In some embodiments, SARS-CoV-2 is an “S-strain” of the virus. In some embodiments, SARS-CoV-2 is an “L-strain” of the virus.

The term “virulence factor” refers to any molecule produced by a virus that enables the virus to achieve at least one of the following: (i) colonization of a niche in the host, including attachment to one or more cells; (ii) evasion of the host's immune response; (ii) inhibition of the host's immune response; and (iii) entry into or exit from one or more cells. Illustrative virulence factors of SARS-CoV-2 include the viral envelope (E) protein, the membrane (M) protein, the nucleocapsid (N) protein, the spike (S) protein, and the 3C-like protease (3CLpro or “main 3CL protease”).

The term “subject,” as used herein, unless otherwise defined, is a mammal, e.g., a human, mouse, rat, guinea pig, dog, cat, horse, cow, pig, or non-human primate, such as a monkey, chimpanzee, or baboon. In some embodiments, the subject is a human. In some embodiments, the subject is an adult human. In some embodiments, the subject is a pediatric human.

As used herein, the term “adult human” refers to a human that is 18 years or older.

As used herein, the term “pediatric human” refers to a human that is about 1 day old to about 18 years old.

The term “straight chain or branched C₁₋₁₀ alkyl group” used herein means an alkyl group having 1-10 carbon atoms which may be straight or branched. Non-limiting examples thereof include methyl, ethyl, propyl, isopropyl, butyl, isobutyl, sec-butyl, tert-butyl, pentyl, isopentyl, neopentyl, tert-pentyl, 1-ethylbutyl, 2-ethylbutyl, 1-propylbutyl, 1,1-dimethylbutyl, 1-isobutyl-3-methylbutyl, 1-ethylpentyl, 1-propylpentyl, 1-isobutylpentyl, 2-ethylpentyl, 2-isopropylpentyl, 2-tert-butylpentyl, 3-ethylpentyl, 3-isopropylpentyl, 4-methylpentyl, 1,4-dimethylpentyl, 2,4-dimethylpentyl, 1-ethyl-4-methylpentyl, hexyl, 1-ethylhexyl, 1-propylhexyl, 2-ethylhexyl, 2-isopropylhexyl, 2-tert-butylhexyl, 3-ethylhexyl, 3-isopropylhexyl, 3-tert-butylhexyl, 4-ethylhexyl, 5-methylhexyl, heptyl, 1-ethylheptyl, 1-isopropylheptyl, 2-ethylheptyl, 2-isopropylheptyl, 3-propylheptyl, 4-propylheptyl, 5-ethylheptyl, 6-methylheptyl, octyl, 1-ethyloctyl, 2-ethyloctyl, nonyl, 1-methylnonyl, 2-methylnonyl, decyl, and the like groups. In one embodiment, a straight chain or branched alkyl group has 1-8 carbon atoms.

The term “C₁₋₄ lower alkyl group” used herein means an alkyl group having 1-4 carbon atoms, and specifically includes methyl, ethyl, propyl, isopropyl, butyl, isobutyl, sec-butyl, tert-butyl, and the like groups.

The term “straight chain or branched C₂₋₁₀ alkenyl group” means an alkenyl group having 2-10 carbon atoms with at least one or more double bonds, which may be straight or branched. Non-limiting examples thereof include allyl, vinyl, isopropenyl, 1-propenyl, 1-methyl-2-propenyl, 2-methyl-2-propenyl, 1-methyl-1-butenyl, crotyl, 1-methyl-3-butenyl, 3-methyl-2-butenyl, 1,3-dimethyl-2-butenyl, 1-pentenyl, 1-methyl-2-pentenyl, 1-ethyl-3-pentenyl, 4-pentenyl, 1,3-pentadienyl, 2,4-pentadienyl, 1-hexenyl, 1-methyl-2-hexenyl, 3-hexenyl, 4-hexenyl, 1-butyl-5-hexenyl, 1,3-hexadienyl, 2,4-hexadienyl, 1-heptenyl, 2-heptenyl, 3-heptenyl, 4-heptenyl, 5-heptenyl, 6-heptenyl, 1,3-heptadienyl, 2,4-heptadienyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 9-decenyl, and the like groups. In one embodiment, an alkenyl group has 2-8 carbon atoms, which may be straight or branched.

The term “halogen atom” means fluorine, chlorine, and bromine atoms.

The term “halo-C₁₋₄ alkyl group” means the above-described C₁₋₄ lower alkyl group substituted with 1-3 halogens, which may be the same or different. Non-limiting examples thereof include fluoromethyl, chloromethyl, bromomethyl, difluoromethyl, dichloromethyl, trifluoromethyl, trichloromethyl, chloroethyl, difluoroethyl, trifluoroethyl, pentachloroethyl, bromopropyl, dichloropropyl, trifluorobutyl, and the like groups. In some embodiments, the C₁₋₄ lower alkyl group is trifluoromethyl and chloroethyl.

The term “C₁₋₄ lower alkoxy group” means the alkoxy group containing the C₁₋₄ lower alkyl group as described above. Examples thereof include methoxy, ethoxy, propoxy, isopropoxy, butoxy, isobutoxy, sec-butoxy, tert-butoxy, and the like groups.

The term “C₁₋₄ lower alkylthio group” means the alkylthio group containing the C₁₋₄ lower alkyl group as described above. Examples thereof include methylthio, ethylthio, propylthio, isopropylthio, butylthio, isobutylthio, sec-butylthio, tert-butylthio, and the like groups.

The term “C₃₋₁₀ cycloalkyl group” means a cycloalkyl group having 3-10 carbon atoms, which may be monocyclic or polycyclic. Examples thereof include cyclopropyl, cyclobutyl, cyclopentyl, cyclohexyl, cycloheptyl, cyclooctyl, octahydroindenyl, decahydronaphthyl, bicyclo[2.2.1]heptyl, adamantyl, and the like groups. In some embodiments, the group has 5-7 carbon atoms, including cyclopentyl, cyclohexyl, and cycloheptyl.

The term “C₅₋₈ cycloalkenyl group” means a cycloalkenyl group having 5-8 carbon atoms with one or more double bonds on the ring. Examples thereof include cyclopentenyl, cyclohexenyl, cycloheptenyl, cyclooctenyl, cyclopentadienyl, cyclohexadienyl, cycloheptadienyl, cyclooctadienyl, and the like groups. In some embodiments, the group has 5-7 carbon atoms, including cyclopentenyl, cyclohexenyl, and cycloheptenyl.

The term “C₃₋₁₀ cycloalkyl C₁₋₁₀ alkyl group” means the above-described straight chain or branched C₁₋₁₀ alkyl group substituted with the above-described C₃₋₁₀ cycloalkyl group. Non-limiting examples thereof include cyclopropylmethyl, cyclopentylmethyl, cyclohexylmethyl, cyclohexyl cyclopentylmethyl, dicyclohexylmethyl, 1-cyclopentylethyl, 1-cyclohexylethyl, 2-cyclopropylethyl, 2-cyclopentylethyl, 2-cyclohexylethyl, 2-cycloheptylethyl, 1-cyclohexyl-1-methylethyl, 1-cyclohexylpropyl, 2-cyclopentylpropyl, 3-cyclobutylpropyl, 3-cyclopentylpropyl, 3-cyclohexylpropyl, 3-cycloheptylpropyl, 1-cyclopropyl-1-methylpropyl, 1-cyclohexyl-2-methylpropyl, 1-cyclopentylbutyl, 1-cyclohexylbutyl, 3-cyclohexylbutyl, 4-cyclopropylbutyl, 4-cyclobutylbutyl, 4-cyclopentylbutyl, 1-cyclohexyl-1-methylbutyl, 1-cyclopentyl-2-ethylbutyl, 1-cyclohexyl-3-methylbutyl, 1-cyclopentylpentyl, 1-cyclohexylpentyl, 1-cyclohexylmethylpentyl, 2-cyclohexylpentyl, 2-cyclohexylmethylpentyl, 3-cyclopentylpentyl, 1-cyclohexyl-4-methylpentyl, 5-cyclopentylpentyl, 1-cyclopentylhexyl, 1-cyclohexylhexyl, 1-cyclopentylmethylhexyl, 2-cyclopentylhexyl, 2-cyclopropylethylhexyl, 3-cyclopentylhexyl, 1-cyclohexylheptyl, 1-cyclopentyl-1-methylheptyl, 1-cyclohexyl-1,6-dimethylheptyl, 1-cycloheptyloctyl, 2-cyclopentyloctyl, 3-cyclohexyloctyl, 2-cyclopentylmethyloctyl, 1-cyclopentylnonyl, 1-cyclohexylnonyl, 3-cyclopropylnonyl, 1-cyclopentyldecyl, 1-cyclohexylundecyl, 1-cyclopentyltridecyl, 2-cyclohexyltridecyl, and the like groups.

The “aryl group” includes phenyl, naphthyl, anthryl, phenanthryl, biphenyl, and the like groups. In some embodiments, the aryl group is a phenyl, naphthyl, or biphenyl.

The “aralkyl group” means the above-described C₁₋₄ lower alkyl group substituted with one or more aryl groups as described above. Examples thereof include benzyl, benzhydryl, trityl, phenethyl, 3-phenylpropyl, 2-phenylpropyl, 4-phenylbutyl, naphthylmethyl, 2-naphthylethyl, 4-biphenylmethyl, 3-(4-biphenyl) propyl, and the like groups.

The “arylalkenyl group” means an alkenyl group having 2-4 carbon atoms substituted with the above-described aryl group. Examples thereof include 2-phenylvinyl, 3-phenyl-2-propenyl, 3-phenyl-2-methyl-2-propenyl, 4-phenyl-3-butenyl, 2-(1-naphthyl)vinyl, 2-(2-naphthyl)vinyl, 2-(4-biphenyl)vinyl, and the like groups.

The “arylthio group” means an arylthio group containing the above-described aryl group and specifically include phenylthio, naphthylthio, and the like groups.

The “heterocyclic ring group” means 5- and 6-membered aromatic or non-aromatic heterocyclic ring groups containing at least one or more, specifically 1-4 or 1-3, hetero atoms selected from nitrogen, oxygen, and sulfur atoms. Non-limiting examples thereof include aromatic heterocyclic rings such as thiatriazolyl, tetrazolyl, dithiazolyl, oxadiazolyl, thiadiazolyl, triazolyl, oxazolyl, pyrazolyl, pyrrolyl, furyl, thienyl, tetrazinyl, triazinyl, pyrazinyl, pyridazinyl, pyrimidinyl, pyridyl, or the like groups and non-aromatic heterocyclic rings such as dioxoranyl, pyrrolidinyl, tetrahydrofuryl, tetrahydrothienyl, dithiadiazinyl, thiadiazinyl, morpholino, morpholinyl, oxazinyl, thiazinyl, piperazinyl, piperidyl, piperidino, pyranyl, thiopyranyl, or the like groups. Non-limiting examples are aromatic heterocyclic (heteroaryl) groups including furyl, thienyl, pyrrolyl, pyridyl, and the like and non-aromatic heterocyclic groups containing at least one nitrogen atom, including pyrrolidinyl, tetrahydrofuryl, piperazinyl, piperidyl, piperidino, and the like groups.

The “heteroarylalkyl group” means the above-described C₁₋₄ lower alkyl group substituted with the above-described 5- or 6-membered aromatic heterocyclic (heteroaryl) group and specifically include 2-thienylmethyl, 2-furylmethyl, 2-pyridylmethyl, 3-pyridylmethyl, 2-thienyl-2-ethyl, 3-furyl-1-ethyl, 2-pyridyl-3-propyl, and the like groups.

The “acyl group” specifically includes formyl, acetyl, propionyl, butyryl, isobutyryl, valeryl, isovaleryl, pivaloyl, hexanoyl, acryloyl, propioloyl, metacryloyl, crotonoyl, benzoyl, naphthoyl, toluoyl, hydroatropoyl, atropoyl, cinnamoyl, furoyl, thenoyl, nicotinoyl, isonicotinoyl, glucoloyl, lactoyl, glyceroyl, tropoyl, benzyloyl, salicyloyl, anisoyl, vaniloyl, veratoroyl, piperoniroyl, protocatechoyl, galloyl, cyclopentanecarbonyl, cyclohexanecarbonyl, cycloheptanecarbonyl, 1-methyl cyclohexanecarbonyl, 1-isopentylcyclopentanecarbonyl, 1-isopentyl cyclohexanecarbonyl, tert-butoxycarbonyl, methoxycarbonyl, ethoxycarbonyl, 2-(1-isopentylcyclohexanecarbonylamino)phenylthiocarbonyl, and the like groups. Non-limiting examples acetyl, tert-butoxycarbonyl, benzoyl, 1-methylcyclohexanecarbonyl, 1-isopentylcyclopentanecarbonyl, 1-isopentylcyclohexanecarbonyl, and 2-(1-isopentylcyclohexanecarbonylamino)phenylthiocarbonyl.

The term “substituted or unsubstituted” of the “substituted or unsubstituted C₃₋₁₀ cycloalkyl group”, the “substituted or unsubstituted C₅₋₈ cycloalkenyl group”, and the “substituted or unsubstituted C₃₋₁₀cycloalkyl C₁₋₁₀ alkyl group” described for R, R₁, and the like means that the group may be substituted with 1-4 substituents which may be the same or different and any position may be arbitrarily substituted without any limitation. Non-limiting examples of these groups are the above-described straight chain or branched C₁₋₁₀ alkyl group; the above-described straight chain or branched C₂₋₁₀ alkenyl group; the above-described C₃₋₁₀ cycloalkyl group; the above-described C₁₋₁₀ cycloalkenyl group; the above-described C₃₋₁₀cycloalkyl C₁₋₁₀ alkyl group; the above-described aryl group; an amino group; a C₁₋₄ lower alkylamino group such as methylamino, ethylamino, or the like groups; an acylamino group such as acetylamino, propionylamino, benzylamino, or the like groups; an oxo group; the above-described aralkyl group; the above-described arylalkenyl group, and the like.

The above substituents are recommended as substituents for R. Among these, non-limiting examples for R₁ are the above-described straight chain or branched C₁₋₁₀ alkyl group, the above-described C₃₋₁₀ cycloalkyl group, the above-described C₅₋₈ cycloalkenyl group, the above-described aryl group, and the above-described amino group.

The term “substituted or unsubstituted” of the “substituted or unsubstituted aryl group”, the “15- or 6-membered heterocyclic group containing 1-3 nitrogen, oxygen, or sulfur atoms”, the “substituted or unsubstituted aralkyl group”, the “substituted or unsubstituted arylalkenyl group”, the “substituted or unsubstituted arylthio group”, and the “substituted or unsubstituted 5- or 6-membered heteroarylalkyl group” described with respect to R, R₁, and the like means that the groups may be substituted with 1-4, or 1-3, substituents which may be the same or different and any position may be arbitrarily substituted without particular restriction. Examples of these groups include the above-described straight chain or branched C₁₋₁₀ alkyl group, including a straight chain or branched C₁₋₆ aralkyl group; the above-described straight chain or branched C₂₋₁₀ alkenyl group, including a straight chain or branched C₂₋₆ alkenyl group; the above-described halogen atom; a nitro group; the above-described amino group that may be substituted with the above-described C₁₋₄ lower alkyl group or the above-described acyl group; a hydroxyl group; the above-described C₁₋₄ lower alkoxy group; the above-described C₁₋₄ lower alkylthio group; the above-described halo-C₁₋₄ lower alkyl group; the above-described acyl group; an oxo group, and the like.

The above substituents are recommended as substituents mainly for R₁. Among these, non-limiting examples for R include the above-described straight chain or branched C₁₋₆ alkyl group, the above-described halogen atom, and a nitro group.

The “substituted or unsubstituted” of the “substituted or unsubstituted straight chain or branched C₁₋₁₀ alkyl group” described for R₁ and the like means that the group may be substituted with 1-3 substituents which may be the same or different and any position may be arbitrarily substituted without particular restriction. Examples of these groups are the above-described C₁₋₄ lower alkoxy group; the above-described C₁₋₄ lower alkyl group; the above-described amino group that may be substituted with an acyl or hydroxyl group; the above-described lower C₁₋₄ alkylthio group; a carbamoyl group; a hydroxyl group; the above-described halogen atom; the above-described acyloxy group containing an acyl group; a carboxyl group; the above-described acyl group; the above-described aryloxy group containing an aryl group that may be substituted; and the like.

The “substituted or unsubstituted” of the “C₁₋₄ lower alkyl group” described with respect to R₂ and the like means that the group may be substituted with 1-3 substituents which may be the same or different and any position may be arbitrarily substituted without particular restriction. Examples of the group include the above-described C₁₋₄ lower alkoxy group; the above-described amino group that may be substituted with the above-described C₁₋₄ lower alkyl group or the above-described acyl group; the above-described C₁₋₄ lower alkylthio group; a carbamoyl group; a hydroxyl group; a carboxyl group; the above-described acyl group; the above-described heterocyclic group (particularly aromatic heterocyclic groups such as thienyl or non-aromatic heterocyclic group such as tetrahydrofuryl); and the like.

The term “substituted or unsubstituted” of the “substituted or unsubstituted amino group” and the “substituted or unsubstituted ureido group” described with respect to R₁ means that the groups may be substituted with one or more, e.g., 1-2, substituents, which may be the same or different and any position may be arbitrarily substituted without particular restriction. Examples of these groups are the above-described C₁₋₄ lower alkyl group; a hydroxyl group; the above-described acyl group; the above-described aryl group which may be substituted with the above-described C₁₋₄ lower alkoxy group; and the like.

The “mercapto-protecting group” described with respect to Z means commonly used mercapto protecting groups. Any organic residues that can be dissociated in vivo may be used without particular restriction. It may form a disulfide structure, that is dimer. Non-limiting examples thereof include C₁₋₄ lower alkoxymethyl; C₁₋₄ lower alkylthiomethyl; aralkyloxymethyl; aralkylthiomethyl; C₃₋₁₀ cycloalkyloxymethyl; C₅₋₈ cycloalkenyloxymethyl; C₃₋₁₀ cycloalkyl C₁₋₁₀ alkoxymethyl; aryloxymethyl; arylthiomethyl; acyl; acyloxy; aminocarbonyloxymethyl; thiocarbonyl; and thio groups. Non-limiting examples thereof include a C₁₋₄ lower alkoxymethyl group with the above-described C₁₋₄ lower alkoxy group; a C₁₋₄ lower alkylthiomethyl group with the above-described C₁₋₄ lower alkylthio group; an aralkyloxymethyl group with the above-described aralkyl group; an aralkylthiomethyl group with the above-described aralkyl group; a C₃₋₁₀ cycloalkyloxymethyl group with the above-described C₃₋₁₀ cycloalkyl group; a C₅₋₈ cycloalkenyloxymethyl group with the above-described C₅₋₈ cycloalkenyl group; a C₃₋₁₀ cycloalkyl C₁₋₁₀ alkoxymethyl group with the above-described C₃₋₁₀ cycloalkyl C₁₋₁₀ alkyl group; an aryloxymethyl group with the above-described aryl group; an arylthiomethyl group with the above-described arylthio group; an acyl group containing the above-described substituted or unsubstituted straight chain or branched C₁₋₁₀ alkyl group, the above-described halo-C₁₋₄ lower alkyl group, the above-described C₁₋₄ lower alkoxy group, the above-described C₁₋₄ lower alkylthio group, the above-described substituted or unsubstituted amino group, the above-described substituted or unsubstituted ureido group, the above-described substituted or unsubstituted C₃₋₁₀ cycloalkyl group, the above-described substituted or unsubstituted C₃₋₁₀ cycloalkyl C₁₋₁₀ alkyl group, the above-described substituted or unsubstituted aryl group, the above-described substituted or unsubstituted aralkyl group, the above-described substituted or unsubstituted arylalkenyl group, the above-described substituted or unsubstituted arylthio group, the above-described substituted or unsubstituted 5- or 6-membered heterocyclic group with 1-3 nitrogen, oxygen, or sulfur atoms, or the above-described substituted or unsubstituted 5- or 6-membered heteroarylalkyl group; an acyloxy group containing the above-described substituted or unsubstituted straight chain or branched C₁₋₁₀ alkyl group, the above-described halo-C₁₋₄ lower alkyl group, the above-described C₁₋₄ lower alkoxy group, the above-described C₁₋₄ lower alkylthio group, the above-described substituted or unsubstituted amino group, the above-described substituted or unsubstituted ureido group, the above-described substituted or unsubstituted C₃₋₁₀ cycloalkyl group, the above-described substituted or unsubstituted C₃₋₁₀ cycloalkyl C₁₋₁₀ alkyl group, the above-described substituted or unsubstituted aryl group, the above-described substituted or unsubstituted aralkyl group, the above-described substituted or unsubstituted arylalkenyl group, the above-described substituted or unsubstituted arylthio group, the above-described substituted or unsubstituted 5- or 6-membered heterocyclic group with 1-3 nitrogen, oxygen, or sulfur atoms, or the above-described substituted or unsubstituted 5- or 6-membered heteroarylalkyl group; an aminocarbonyloxymethyl group that may be substituted with the above-described substituted or unsubstituted straight chain or branched C₁₋₁₀ alkyl group, the above-described halo-C₁₋₄ alkyl group, the above-described C₁₋₄ lower alkoxy group, the above-described C₁₋₄ lower alkylthio group, the above-described substituted or unsubstituted C₃₋₁₀ cycloalkyl group, the above-described substituted or unsubstituted C₃₋₁₀cycloalkyl C₁₋₁₀ alkyl group, the above-described substituted or unsubstituted aryl group, the above-described substituted or unsubstituted aralkyl group, the above-described substituted or unsubstituted arylalkenyl group, the above-described substituted or unsubstituted 5- or 6-membered heterocyclic group with 1-3 nitrogen, oxygen, or sulfur atoms, or the above-described substituted or unsubstituted 5- or 6-membered heteroarylalkyl group; a thiocarbonyl group containing the above-described substituted or unsubstituted straight chain or branched C₁₋₁₀ alkyl group, the above-described halo-C₁₋₄ lower alkyl group, the above-described C₁₋₄ lower alkoxy group, the above-described C₁₋₄ lower alkylthio group, the above-described substituted or unsubstituted amino group, the above-described substituted or unsubstituted ureido group, the above-described substituted or unsubstituted C₃₋₁₀ cycloalkyl group, the above-described substituted or unsubstituted C₃₋₁₀ cycloalkyl C₁₋₁₀ alkyl group, the above-described substituted or unsubstituted aryl group, the above-described substituted or unsubstituted aralkyl group, the above-described substituted or unsubstituted arylalkenyl group, the above-described substituted or unsubstituted arylthio group, the above-described substituted or unsubstituted 5- or 6-membered heterocyclic group with 1-3 nitrogen, oxygen, or sulfur atoms, or the above-described substituted or unsubstituted 5- or 6-membered heteroarylalkyl group; and a thio group containing the above-described substituted or unsubstituted C₁₋₄ lower alkyl or aryl group.

In some embodiments, the “straight chain or branched C₁₋₁₀ alkyl group” for R is methyl, ethyl, isopropyl, butyl, isobutyl, tert-butyl, heptyl, 1-propylbutyl, or 1-isobutyl-3-methylbutyl.

In some embodiments, the “straight chain or branched C₂₋₁₀ alkenyl group” referred to herein as R includes the non-limiting group selected from allyl, vinyl, isopropenyl, 1-methyl-2-propenyl, 2-methyl-2-propenyl, 1-methyl-1-butenyl, crotyl, 1,3-dimethyl-2-butenyl, 1-pentenyl, and 1-methyl-2-pentenyl.

The “halo-C₁₋₄ lower alkyl group” for R means a C₁₋₄ lower alkyl group, e.g., a methyl group, substituted with the above-described halogen atoms, including fluorine and chlorine. In some embodiments, the “halo-C₁₋₄ lower alkyl group is a trifluoromethyl group.

The “substituted or unsubstituted C₃₋₁₀ cycloalkyl group” for R means a C₃-10 cycloalkyl group (including cyclopropyl, cyclobutyl, cyclopentyl, cyclohexyl, cycloheptyl, octahydroindenyl, decahydronaphthyl, adamantyl, and bicyclo[2.2.1]heptyl) that may be substituted with 1-4 substituents selected from the above-described straight chain or branched C₁₋₁₀ alkyl group, (including a C₁₋₈ alkyl group such as methyl, ethyl, propyl, isopropyl, butyl, isobutyl, tert-butyl, pentyl, isopentyl, 2,2-dimethylpropyl, 4-methylpentyl, 2-ethylbutyl, or the like), the above-described straight chain or branched C₂₋₁₀ alkenyl group (including a C₂₋₈ alkenyl group such as 1-methylvinyl, 2-methylvinyl, 3-methyl-3-propenyl, or the like), the above-described C₃₋₁₀cycloalkyl group (including a C₃-7 cycloalkyl group such as cyclopropyl, cyclopentyl, cyclohexyl, or the like), the above-described C₅₋₈ cycloalkenyl group (including a C₅₋₆ cycloalkenyl group such as cyclopentenyl, cyclohexenyl, or the like), the above-described C₃₋₁₀ cycloalkyl C₁₋₁₀ alkyl group (including a C₃₋₇ cycloalkyl C₁₋₄ alkyl group such as cyclopropylmethyl, 2-cyclopropylethyl, 2-cyclopentylethyl, cyclohexylmethyl, 2-cyclohexylethyl, or the like), the above-described aryl group (including a phenyl group), an oxo group, the above described aralkyl group (including a phenyl C₁₋₄ lower alkyl group such as benzyl, phenethyl, or the like), and the above-described arylalkenyl group (including a 2-phenylvinyl group). Non-limiting examples thereof include 2,2,3,3-tetramethylcyclopropyl, 1-isopentylcyclobutyl, 1-isopropylcyclopentyl, 1-isobutylcyclopentyl, 1-isopentylcyclopentyl, 1-cyclohexylmethylcyclopentyl, cyclohexyl, 1-methylcyclohexyl, 1-ethylcyclohexyl, 1-propylcyclohexyl, 1-isopropylcyclohexyl, 1-butylcyclohexyl, 1-isobutylcyclohexyl, 1-pentylcyclohexyl, 1-isopentylcyclohexyl, 1-(2,2-dimethylpropyl)cyclohexyl, 1-(4-methylpentyl)cyclohexyl, 1-(2-ethylbutyl) cyclohexyl, 4-tert-butyl-1-isopentylcyclohexyl, 1-cyclopropylcyclohexyl, 1-bicyclohexyl, 1-phenylcyclohexyl, 1-cyclopropylmethylcyclohexyl, 1-cyclohexylmethylcyclohexyl, 1-(2-cyclopropylethyl) cyclohexyl, 1-(2-cyclopentylethyl)cyclohexyl, 1-(2-cyclohexylethyl)cyclohexyl, 4-methylcyclohexyl, 4-propylcyclohexyl, 4-isopropylcyclohexyl, 4-tert-butylcyclohexyl, 4-pentylcyclohexyl, 4-bicyclohexyl, 1-isopentylcycloheptyl, 3a-octahydroindenyl, 4a-decahydronaphthyl, 1-adamantyl, and 7,7-dimethyl-1-(2-oxo)-bicyclo[2.2.1]heptyl. The site of substitution is not specifically limited, but including at position 1. Any substitution group as described above may be used. In some embodiments, the substitution group is a straight chain or branched C₁₋₁₀ alkyl group.

The substituent for the “substituted or unsubstituted C₅₋₈ cycloalkenyl group” for R is the same as that for the above “substituted or unsubstituted C₃₋₁₀ cycloalkyl group”. Specifically, it means a cycloalkenyl group (especially cyclopentenyl and cyclohexenyl) that may have 1-4 substituents selected from the above-described straight chain or branched C₁₋₁₀ alkyl group (including a C₁₋₈ alkyl group such as methyl, ethyl, propyl, isopropyl, butyl, isobutyl, pentyl, isopentyl, 2,2-dimethylpropyl, 4-methylpentyl, or the like), the above-described straight chain or branched C₂₋₁₀ alkenyl group (including a C₂₋₈alkenyl group such as 1-methylvinyl, 2-methylvinyl, 3-methyl-3-propenyl, and the like), the above-described C₃₋₁₀cycloalkyl group (including a C₃₋₇cycloalkyl group such as cyclopropyl, cyclopentyl, cyclohexyl, or the like), the above-described C₅₋₈ cycloalkenyl group (including a C₅₋₆ cycloalkenyl group like cyclopentenyl, cyclohexenyl, or the like), the above-described C₃₋₁₀ cycloalkyl C₁₋₁₀ alkyl group (including a C₃₋₇cycloalkyl C₁₋₄ lower alkyl group such as cyclopropyl methyl, 2-cyclopropylethyl, 2-cyclopentylethyl, cyclohexylmethyl, 2-cyclohexylethyl, or the like), the above-described aryl group (including a phenyl group), an oxo group, the above-described aralkyl group (including a phenyl C₁₋₄ lower alkyl group such as benzyl, phenethyl, or the like), and arylalkenyl group (including 2-phenylvinyl). Non-limiting examples of the cycloalkenyl group includes 1-isopropyl-2-cyclopentenyl, 1-isopropyl-3-cyclopentenyl, 1-isobutyl-2-cyclopentenyl, 1-isobutyl-3-cyclopentenyl, 1-isopentyl-2-cyclopentenyl, 1-isopentyl-3-cyclopentenyl, 1-cyclohexylmethyl-2-cyclopentenyl, 1-cyclohexylmethyl-3-cyclopentenyl, 1-cyclohexenyl, 2-cyclohexenyl, 3-cyclohexenyl, 1-methyl-2-cyclohexenyl, 1-methyl-3-cyclohexenyl, 1-ethyl-2-cyclohexenyl, 1-ethyl-3-cyclohexenyl, 1-propyl-2-cyclohexenyl, 1-propyl-3-cyclohexenyl, 1-isopropyl-2-cyclohexenyl, 1-isopropyl-3-cyclohexenyl, 1-butyl-2-cyclohexenyl, 1-butyl-3-cyclohexenyl, 1-isobutyl-2-cyclohexenyl, 1-isobutyl-3-cyclohexenyl, 1-pentyl-2-cyclohexenyl, 1-pentyl-3-cyclohexenyl, 1-isopentyl-2-cyclohexenyl, 1-isopentyl-3-cyclohexenyl, 1-(2,2-dimethylpropyl)-2-cyclohexenyl, 1-(2,2-dimethylpropyl)-3-cyclohexenyl, 1-(4-methylpentyl)-2-cyclohexenyl, 1-(4-methylpentyl)-3-cyclohexenyl, 1-cyclopropyl-2-cyclohexenyl, 1-cyclopropyl-3-cyclohexenyl, 1-cyclohexyl-2-cyclohexenyl, 1-cyclohexyl-3-cyclohexenyl, 1-phenyl-2-cyclohexenyl, 1-phenyl-3-cyclohexenyl, 1-cyclopropylmethyl-2-cyclohexenyl, 1-cyclo propylmethyl-3-cyclohexenyl, 1-cyclohexylmethyl-2-cyclohexenyl, 1-cyclohexylmethyl-3-cyclohexenyl, 1-(2-cyclopropylethyl)-2-cyclohexenyl, 1-(2-cyclopropylethyl)-3-cyclohexenyl, 1-(2-cyclopentylethyl)-2-cyclohexenyl, 1-(2-cyclopentylethyl)-3-cyclohexenyl, 1-(2-cyclohexylethyl)-2-cyclohexenyl, and 1-(2-cyclohexylethyl)-3-cyclohexenyl. There is no special restriction on the substitution position, but in some embodiments, the substitution position is position 1. Any one of the above substituents may be used. In some embodiments, the substituent is a straight chain or branched C₁₋₁₀ alkyl group or the C₃-10 cycloalkyl C₁₋₄ alkyl group.

The “substituted or unsubstituted C₃₋₁₀ cycloalkyl C₁₋₁₀ alkyl group” for R means a C₃₋₁₀ cycloalkyl C₁₋₁₀ alkyl group (including cyclohexylmethyl, 1-cyclohexylethyl, 1-cyclohexyl-1-methylethyl, 1-cyclohexyl-2-methylpropyl, 1-cyclohexyl-3-methylbutyl, 1-cyclohexylhexyl, 1-cyclohexyl-4-methylpentyl, and 1-cyclohexylheptyl) C₁₋₁₀ alkyl group of which is straight chain or branched and which may have 1-4 substituents selected from the above-described C₃₋₁₀cycloalkyl group (including a C₃₋₇cycloalkyl group such as cyclopentyl or cyclohexyl), the above-described C₅₋₈ cycloalkenyl group (including a C₅₋₇ cycloalkenyl group such as cyclopentenyl or cyclohexenyl), and the above-described aryl group (including a phenyl group). There is no special restriction on the substitution position. The above-described substituents may be placed at the straight chain or branched C₁₋₁₀ alkyl moiety. Non-limiting examples of the C₃₋₁₀ cycloalkyl C₁₋₁₀ alkyl group include cyclohexylmethyl, 1-cyclohexylethyl, cyclohexylcyclo-pentylmethyl, dicyclohexylmethyl, 1-cyclohexyl-1-methylethyl, 1-cyclohexyl-2-methylpropyl, 1-cyclohexyl-3-methylbutyl, 1-cyclohexyl-4-methylpentyl, 1-cyclohexylhexyl, and 1-cyclohexylheptyl.

The “substituted or unsubstituted aryl group” for R means an aryl group (including a phenyl group) that may have 1-4 substituents selected from the above-described straight chain or branched C₁₋₆ alkyl group (including a tert-butyl group), the above-described halogen atom (including fluorine and chlorine), and a nitro group. Non-limiting examples of the aryl group are phenyl, 2-chlorophenyl, 4-nitrophenyl, and 3,5-di-tert-butylphenyl.

The “substituted or unsubstituted aralkyl” for R means an aralkyl group (including benzyl, benzhydryl, and trityl) which may have substituents selected from the above-described halogen atom (including fluorine and chlorine), a nitro group, and a hydroxy group, and in which the C₁₋₄ lower alkyl group is straight chain or branched. There is no special restriction on the position of substitution. The straight chain or branched C₁-4 lower alkyl moiety may be substituted. Non-limiting examples of the aralkyl group are benzyl and trityl.

The “substituted or unsubstituted 5- or 6-membered heterocyclic group having 1-3 nitrogen, oxygen or sulfur atoms” for R means the above-described heterocyclic group that may have 1-4 substituents selected from the above-described straight chain or branched C₁₋₆ alkyl group (including a tert-butyl group), the above-described halogen atom (including fluorine and chlorine), and a nitro group. In some embodiments, the heterocyclic group is selected an aromatic heterocyclic group, including furyl, thienyl, and pyridyl.

The “substituted or unsubstituted straight chain or branched C₁₋₁₀ alkyl group” for R₁ means a straight chain or branched C₁₋₁₀ alkyl group that may have a substituent selected from the above-described halogen atom (including fluorine and chlorine), the above-described C₁₋₄ lower alkoxy group (including a methoxy group), an amino group that may be substituted with the above-described C₁₋₄ lower alkyl group (including a methyl group), the above-described acyl group (including an acetyl group), or a hydroxyl group, the above-described C₁₋₄ lower alkylthio group (including a methylthio group), a carbamoyl group, a hydroxyl group, an acyloxy group having the above-described acyl group (including an acetyloxy group), a carboxyl group, an acyl group (including a methoxycarbonyl group), and an aryloxy group having the above-described substituted or unsubstituted aryl group (including a phenoxy group and a 4-chlorophenoxy group). Non-limiting examples of the alkyl group include methyl, chloromethyl, ethyl, isopropyl, 1-methyl-2-pentyl, octyl, methoxymethyl, dimethylaminomethyl, acetylaminomethyl, 1-acetyl aminoethyl, 1-acetylamino-2-methylpropyl, 1-acetylamino-3-methylbutyl, 1-acetylamino-3-methylthiopropyl, 1-acetylamino-3-carbamoylpropyl, 1-hydroxy-1-methylethyl, 1-acetyloxy-1-methylethyl, 4-carboxybutyl, 2-methoxycarbonylethyl, phenoxymethyl, and 4-chlorophenoxymethyl.

In some embodiments, the “C₁₋₄ lower alkoxy group” for R₁ is a methoxy group or a tert-butoxy group.

In some embodiments, the “C₁₋₄ lower alkylthio group” for R₁ is a methylthio group.

The “substituted or unsubstituted amino group” for R₁ means an amino group that may have a substituent selected from the above-described C₁₋₄ lower alkyl group (including ethyl, isopropyl, and tert-butyl), the above-described acyl group (including acetyl and benzoyl), and the above-described aryl group (including phenyl and 4-methoxyphenyl) that may be substituted with the above-described C₁₋₄ lower alkoxy group. Non-limiting examples of the amino group are ethylamino, isopropylamino, tert-butylamino, phenylamino, and 4-methoxyphenylamino.

The “substituted or unsubstituted ureido group” for R₁ means a ureido group that may have a substituent selected from the above-described C₁₋₄ lower alkyl group (including methyl and ethyl), the above-described acyl group (including acetyl and benzoyl), and the above-described aryl group (including phenyl and 4-methoxyphenyl) that may be substituted with the above-described C₁₋₄ lower alkoxy group. In some embodiments the ureido group is a N,N′-diphenylureido group.

The “substituted or unsubstituted C₃₋₁₀ cycloalkyl group” for R₁ means a C₃-10 cycloalkyl group (including cyclopropyl and cyclohexyl) that may have a substituent selected from the above-described straight chain or branched C₁₋₁₀ alkyl group (including methyl, tert-butyl, and isopentyl), an amino group, an amino group (including methylamino, ethylamino, acetylamino, and benzylamino) that may be substituted with the above-described C₁₋₄ lower alkyl or acyl groups. Non-limiting examples of the cycloalkyl group are cyclopropyl, cyclohexyl, 1-methylcyclohexyl, 1-isopentylcyclohexyl, 1-aminocyclohexyl, 1-acetylaminocyclohexyl, and 4-tert-butylcyclohexyl.

The “substituted or unsubstituted C₃₋₁₀ cycloalkyl C₁₋₁₀ alkyl group” for R₁ means a C₃₋₁₀ cycloalkyl C₁₋₁₀ alkyl group which may have a substituent selected from the above-described C₃₋₁₀ cycloalkyl group (including cyclopentyl and cyclohexyl), the above-described C₅₋₈ cycloalkenyl group (including cyclopentenyl and cyclohexenyl), and the above-described aryl group (including a phenyl group) and in which the C₁₋₁₀ alkyl moiety is straight chain or branched. There is no special restriction on the position of substitution. The straight chain or branched C₁₋₁₀ alkyl moiety may be substituted. In some embodiments, the cyclohexylmethyl group is a C₃₋₁₀ cycloalkyl C₁₋₁₀ alkyl group.

The “substituted or unsubstituted aryl group” for R₁ means an aryl group (including phenyl and naphthyl) that may have a substituent selected from the above-described straight chain or branched C₁₋₆ alkyl group (including methyl and tert-butyl group), the above-described halogen atom (including fluorine and chlorine), a nitro group, a hydroxyl group, the above-described C₁₋₄ lower alkoxy group (including a methoxy group), and the above-described acyl group (including a 2-(1-isopentylcyclohexanecarbonylamino)phenylthiocarbonyl group). Non-limiting examples of the aryl group include phenyl, 1-naphthyl, 2-naphthyl, 2-chlorophenyl, 2,6-dichlorophenyl, 2,6-dimethylphenyl, 2-methoxyphenyl, 2-nitrophenyl, 4-nitrophenyl, 3,5-di-tert-butyl-4-hydroxyphenyl, and 4-[2-(1-isopentylcyclohexanecarbonylamino)phenylthiocarbonyl]phenyl.

The “substituted or unsubstituted aralkyl group” for R₁ means an aralkyl group (including benzyl, phenethyl, 3-phenylpropyl, naphthylmethyl, and biphenylmethyl) that may have a substituent selected from the above-described halogen atom (including fluorine and chlorine), a nitro group, an amino group (including amino, acetylamino, pivaloylamino, 1-methylcyclohexanecarbonyl-amino, tert-butoxycarbonylamino, and benzoylamino) that may be substituted with the above-described C₁₋₄ lower alkyl group or the above-described acyl group, and a hydroxyl group, and in which the C₁₋₄ lower alkyl group are straight chain or branched. There is no special restriction on the position of substitution. The straight chain or branched C₁₋₄ lower alkyl moiety may be substituted. Non-limiting examples of the aralkyl group include benzyl, phenethyl, 3-phenylpropyl, 2-naphthylmethyl, 4-biphenylmethyl, benzhydryl, 2-chlorophenylmethyl, 3-chlorophenylmethyl, 4-chlorophenylmethyl, 2-nitrophenylmethyl, 4-nitrophenylmethyl, 2-pivaloylaminophenylmethyl, 2-(1-methylcyclohexanecarbonylamino)phenylmethyl, 2-tert-butoxy-carbonylaminophenylmethyl, 3-acetylaminophenylmethyl, 3-(1-methylcyclohexanecarbonylamino)phenylmethyl, α-aminobenzyl, α-acetylaminobenzyl, α-(1-methylcyclohexanecarbonylamino)benzyl, α-benzoylaminobenzyl, α-aminophenethyl, α-acetylaminophenethyl, and 1-acetylamino-2-(4-hydorxyphenyl) ethyl.

The “substituted or unsubstituted arylalkenyl group” for R₁ means an arylalkenyl group (particularly phenylvinyl) that may have a substituent selected from the above-described straight chain or branched C₁₋₆ lower alkyl group (including methyl and tert-butyl), the above-described halogen atom (including fluorine and chlorine), a nitro group, and a hydroxyl group, with a 2-phenylvinyl group being among the non-limiting options.

The “substituted or unsubstituted arylthio group” for R₁ means an arylthio group (including a phenylthio group) that may have a substituent selected from the above-described halogen atom (including fluorine and chlorine), a nitro group, and an amino group that may be substituted with the above-described C₁₋₄ lower alkyl group or the above-described acyl group (including amino, acetylamino, pivaloylamino, 1-methylcyclohexanecarbonylamino, and benzoylamino), a hydroxyl group, and the above-described halo-C₁₋₄ lower alkyl group (including a trifluoromethyl group). Non-limiting examples of the arylthio group include phenylthio, 2-pivaloylaminophenylthio, 2-(1-methylcyclohexanecarbonylamino)phenylthio, and 2-(1-methyl cyclohexanecarbonylamino-4-trifluoromethyl)phenylthio.

The “substituted or unsubstituted 5- or 6-membered heterocyclic ring groups with 1-3 nitrogen, oxygen, or sulfur atoms” for R₁ means heterocyclic ring groups (including an aromatic heterocyclic group such as pyridyl or a non-aromatic heterocyclic group such as piperidyl or pyrrolidinyl) that may have substituents selected from the above-described straight chain or branched C₁₋₆ alkyl group (including a methyl group), a halogen atom (including fluorine and chlorine), the above-described acyl group (including acetyl and benzoyl), and an oxo group. Non-limiting examples thereof are 3-pyridyl, 1-methyl-4-piperidyl, 1-acetyl-4-piperidyl, 5-oxo-2-pyrrolidinyl, 1-acetyl-2-pyrrolidinyl, and 1-benzoyl-2-pyrrolidinyl. In some embodiments, the 4-piperidyl group includes a 1-methyl-4-piperidyl or 1-acetyl-4-piperidyl group.

The “substituted or unsubstituted 5- or 6-membered heteroarylalkyl group” for R₁ means the above-described heteroarylalkyl group (including a 2-tenyl group) that may be substituted with the above-described straight chain or branched C₁₋₆ alkyl group (including a methyl group) and the above-described halogen atom (including fluorine and chlorine). In some embodiments, the heteroarylalkyl is a 2-tenyl group.

The “substituted or unsubstituted C₁₋₄ lower alkyl group” for R₂ means a C₁₋₄ lower alkyl group (including a methyl group) that may have 1-3 substituents selected from the above-described C₁₋₄ lower alkoxy group (including a methoxy group), an amino group that may be substituted with the above-described C₁₋₄ lower alkyl or acyl group (including a dimethylamino group), the above-described C₁₋₄ lower alkylthio group (including a methylthio group), a carbamoyl group, a hydroxyl group, a carboxyl group, the above-described acyl group (including a methoxycarbonyl group), and the above-described heterocyclic group (including an aromatic heterocyclic group such as thienyl or a non-aromatic heterocyclic group such as tetrahydrofuryl). In some embodiments, the non-aromatic heterocyclic group is a tetrahydrofurylmethyl group.

The “substituted or unsubstituted aryl group” for R₂ is the same as that for R₁. Non-limiting examples thereof are a phenyl group, a halogenated phenyl group, an acylamino-substituted phenyl group, and the like.

The “halogen atom” for X₁, X₂, X₃, and X₄ means a halogen atom including fluorine, chlorine, bromine, and the like, with fluorine and chlorine being options in one embodiment.

In some embodiments, the “C₁₋₄ lower alkyl group” for X₁, X₂, X₃, and X₄ is a methyl group.

The “halo-C₁₋₄ lower alkyl group” for X₁, X₂, X₃, and X₄ means a C₁₋₄ lower alkyl group (including a methyl group) substituted with the above-described halogen atom (including fluorine and chlorine). In some embodiments, the halo-C₁₋₄ lower alkyl group is a trifluoromethyl group.

In some embodiments, the “C₁₋₄ lower alkoxy group” for X₁, X₂, X₃, and X₄ is a methoxy group.

In some embodiments, the “acyl group” for X₁, X₂, X₃, and X₄ is a benzoyl group.

In some embodiments, the “aryl group” for X₁, X₂, X₃, and X₄ is a phenyl group.

The “1-substituted-C₃₋₁₀cycloalkyl group” for R″ means a cycloalkyl group (for example, cyclopropyl, cyclobutyl, cyclopentyl, cyclohexyl, and cycloheptyl, including a C₅-7 cycloalkyl group, including a cyclohexyl group) that is substituted at position 1 with substituents selected from the above-described straight chain or branched C₁₋₁₀ alkyl group (including a C₁₋₈ alkyl group such as methyl, ethyl, propyl, isopropyl, butyl, isobutyl, pentyl, isopentyl, 2,2-dimethylpropyl, 4-methylpentyl, or 2-ethylbutyl), the above-described straight chain or branched C₂₋₁₀ alkenyl group (including a C₂₋₈ alkenyl group such as 1-methylvinyl, 2-methylvinyl, or 3-methyl-3-propenyl), the above-described C₃-10 cycloalkyl (including a C₃₋₇ cycloalkyl group such as cyclopropyl, cyclopentyl, or cyclohexyl), the above-described C₅₋₈ cycloalkenyl group (including a C₅₋₆ cycloalkenyl group such as cyclopentenyl or cyclohexenyl), the above-described C₃₋₁₀cycloalkyl C₁₋₁₀ alkyl group (including a C₃₋₇ cycloalkyl C₁₋₄ lower alkyl group such as cyclopropylmethyl, 2-cyclopropylethyl, 2-cyclopentylethyl, cyclohexylmethyl, or 2-cyclohexylethyl), the above-described aryl group (including a phenyl group), the above-described aralkyl group (including a phenyl C₁₋₄ lower alkyl group such benzyl and phenethyl), and an arylalkenyl group (including 2-phenylvinyl). Non-limiting examples of the 1-substituted-C₃₋₁₀ cycloalkyl group include 1-isopentylcyclobutyl, 1-isopropylcyclopentyl, 1-isobutylcyclopentyl, 1-isopentyl cyclopentyl, 1-cyclohexylmethylcyclopentyl, 1-methylcyclohexyl, 1-ethylcyclohexyl, 1-propylcyclohexyl, 1-isopropylcyclohexyl, 1-butylcyclohexyl, 1-isobutylcyclohexyl, 1-pentylcyclohexyl, 1-isopentylcyclohexyl, 1-(2,2-dimethylpropyl)cyclohexyl, 1-(4-methylpentyl)cyclohexyl, 1-(2-ethylbutyl)cyclohexyl, 1-cyclopropylcyclohexyl, 1-bicyclohexyl, 1-phenylcyclohexyl, 1-cyclopropylmethylcyclohexyl, 1-cyclohexylmethylcyclohexyl, 1-(2-cyclopropylethyl)cyclohexyl, 1-(2-cyclopentylethyl)cyclohexyl, 1-(2-cyclohexylethyl)cyclohexyl, and 1-isopentylcycloheptyl. In some embodiments, the straight chain or branched C₁₋₁₀ alkyl group is selected as a substituent at position 1.

The “1-substituted-C₅₋₈ cycloalkenyl group” for R″ means a cycloalkenyl groups (including a C₅₋₆ cycloalkenyl group such as cyclopentenyl or cyclohexenyl) that is substituted at position 1 with substituents selected from the above-described straight chain or branched C₁₋₁₀ alkyl group (including a C₁₋₈ alkyl group such as methyl, ethyl, propyl, isopropyl, butyl, isobutyl, pentyl, isopentyl, 2,2-dimethyl propyl, and 4-methylpentyl), the above-described straight chain or branched C₂₋₁₀ alkenyl group (including a C₂₋₈ alkenyl group such as 1-methylvinyl, 2-methylvinyl, or 3-methyl-3-propenyl), the above-described C₃₋₁₀cycloalkyl group (including a C₃₋₇cycloalkyl group such as cyclopropyl, cyclopentyl, or cyclohexyl), the above-described C₅₋₈ cycloalkenyl group (including a C₅₋₆ cycloalkenyl group such as cyclopentenyl or cyclohexenyl), the above-described C₃₋₁₀ cycloalkyl C₁₋₁₀ alkyl group (including a C₃₋₇ cycloalkyl C₁₋₄ lower alkyl group such as cyclopropylmethyl, 2-cyclopropylethyl, 2-cyclopentylethyl, cyclohexylmethyl, or 2-cyclohexylethyl), the above-described aryl group (including a phenyl group), the above-described aralkyl group (including a phenyl C₁₋₄ lower alkyl group such as benzyl or phenethyl), and the above-described arylalkenyl group (including a 2-phenylvinyl group). Non-limiting examples of the 1-substituted-C₅₋₈ cycloalkenyl group include 1-isopropyl-2-cyclopentenyl, 1-isopropyl-3-cyclopentenyl, 1-isobutyl-2-cyclopentenyl, 1-isobutyl-3-cyclopentenyl, 1-isopentyl-2-cyclopentenyl, 1-isopentyl-3-cyclopentenyl, 1-cyclohexylmethyl-2-cyclopentenyl, 1-cyclohexylmethyl-3-cyclopentenyl, 1-methyl-2-cyclohexenyl, 1-methyl-3-cyclohexenyl, 1-ethyl-2-cyclohexenyl, 1-ethyl-3-cyclohexenyl, 1-propyl-2-cyclohexenyl, 1-propyl-3-cyclohexenyl, 1-isopropyl-2-cyclohexenyl, 1-isopropyl-3-cyclohexenyl, 1-butyl-2-cyclohexenyl, 1-butyl-3-cyclohexenyl, 1-isobutyl-2-cyclohexenyl, 1-isobutyl-3-cyclohexenyl, 1-pentyl-2-cyclohexenyl, 1-pentyl-3-cyclohexenyl, 1-isopentyl-2-cyclohexenyl, 1-isopentyl-3-cyclohexenyl, 1-(2,2-dimethylpropyl)-2-cyclohexenyl, 1-(2,2-dimethylpropyl)-3-cyclohexenyl, 1-(4-methylpentyl)-2-cyclohexenyl, 1-(4-methylpentyl)-3-cyclohexenyl, 1-cyclopropyl-2-cyclohexenyl, 1-cyclopropyl-3-cyclohexenyl, 1-cyclohexyl-2-cyclohexenyl, 1-cyclohexyl-3-cyclohexenyl, 1-phenyl-2-cyclohexenyl, 1-phenyl-3-cyclohexenyl, 1-cyclopropylmethyl-2-cyclohexenyl, 1-cyclopropylmethyl-3-cyclohexenyl, 1-cyclohexylmethyl-2-cyclohexenyl, 1-cyclohexylmethyl-3-cyclohexenyl, 1-(2-cyclopropylethyl)-2-cyclohexenyl, 1-(2-cyclopropylethyl)-3-cyclohexenyl, 1-(2-cyclopentylethyl)-2-cyclohexenyl, 1-(2-cyclopentylethyl)-3-cyclohexenyl, 1-(2-cyclohexylethyl)-2-cyclohexenyl, and 1-(2-cyclohexylethyl)-3-cyclohexenyl. In some embodiments, the straight chain or branched C₁₋₁₀ alkyl group is an option as a substituent at position 1.

### Antiviral Compounds

In some embodiments, the antiviral compound is dalcetrapib or a pharmaceutically acceptable salt, hydrate or solvate thereof.

Dalceptrapib has a chemical name S-[2-({[1-(2-Ethylbutyl)cyclohexyl]carbonyl}amino)phenyl]-2-methylpropanethioate, and is also known as JTT-705 or CAS 211513-37-0. The structure of dalcetrapib is shown below:

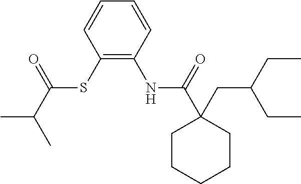 [SMILES: CCC(CC)CC1(C(=O)Nc2ccccc2SC(=O)C(C)C)CCCCC1]

In some embodiments, dalcetrapib is a solid in crystalline or amorphous form. In some embodiments, dalcetrapib has a crystalline form A as disclosed in WO2012/069087. Form A is characterized by an X-ray powder diffraction pattern having peaks at about 7.9°, 8.5°, 11.7°, 12.7°, 17.1°, 18.0°, 18.5°, 20.2°, 22.1°, 24.7°+0.2°, particularly by XRPD peaks observed at an angle of diffraction 2Theta of 7.9°, 11.7°, 17.10, 18.5° (±0.2°).

In some embodiments, the antiviral compound is dalcetrapib thiol or a pharmaceutically acceptable salt, hydrate or solvate thereof. (See Black et al., Clinical Pharmacokinetics, 57(11) 1359-1367 (2018)). The structure of dalcetrapib thiol is depicted below:

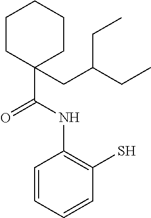 [SMILES: CCC(CC)CC1(C(=O)Nc2ccccc2S)CCCCC1]

In some embodiments, the antiviral compound is dalcetrapib thiol dimer or a pharmaceutically acceptable salt, hydrate or solvate thereof
(See Black et al., Clinical Pharmacokinetics, 57(11) 1359-1367 (2018)). The structure of dalcetrapib thiol dimer is depicted below:

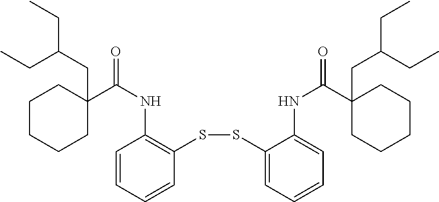 [SMILES: CCC(CC)CC1(C(=O)Nc2ccccc2SSc2ccccc2NC(=O)C2(CC(CC)CC)CCCCC2)CCCCC1]

In some embodiments, the antiviral compound is a compound of Formula (I):

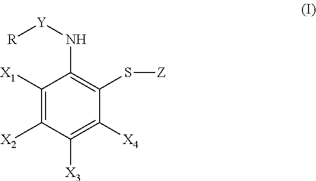

or a pharmaceutically acceptable salt, hydrate, or solvate thereof, wherein:

R represents

a straight chain or branched C₁₋₁₀ alkyl group;

a straight chain or branched C₂₋₁₀ alkenyl group;

a halo-C₁₋₄ lower alkyl group;

a substituted or unsubstituted C₃₋₁₀ cycloalkyl group;

a substituted or unsubstituted C₅₋₈ cycloalkenyl group;

a substituted or unsubstituted C₃₋₁₀ cycloalkyl C₁₋₁₀ alkyl group;

a substituted or unsubstituted aryl group;

a substituted or unsubstituted aralkyl group; or

a substituted or unsubstituted 5- or 6-membered heterocyclic group having 1-3 nitrogen, oxygen or sulfur atoms,

X₁, X₂, X₃, and X₄ may be the same or different and represents

a hydrogen atom;

a halogen atom;

a C₁₋₄ lower alkyl group;

a halo-C₁₋₄ lower alkyl group;

a C₁₋₄ lower alkoxy group;

a cyano group;

a nitro group;

an acyl group; or

an aryl group,

Y represents

—CO—; or

—S₂, and

Z represents

a hydrogen atom; or

a mercapto-protecting group,

or, a pharmaceutically acceptable salt, hydrate, or solvate thereof.

In some embodiments, an antiviral compound for use in the compositions and methods disclosed herein may be any one of the compounds described below.

In some embodiments, the antiviral compound may be a compound of Formula (I), wherein:

R represents

a straight chain or branched C₁₋₁₀ alkyl group;

a straight chain or branched C₂₋₁₀ alkenyl group;

a halo-C₁₋₄ lower alkyl group substituted with 1-3 halogen atoms selected from fluorine, chlorine, and bromine;

a C₃₋₁₀ cycloalkyl group, a C₅₋₈ cycloalkenyl group, or a C₃₋₁₀ cycloalkyl C₁₋₁₀ alkyl group, each of which may have 1-4 substituents selected from

a straight chain or branched C₁₋₁₀ alkyl group,

a straight chain or branched C₂₋₁₀ alkenyl group,

a C₃₋₁₀ cycloalkyl group,

a C₅₋₈ cycloalkenyl group,

a C₃₋₁₀cycloalkyl C₁₋₁₀ alkyl group,

an aryl group selected from phenyl, biphenyl, and naphthyl,

an oxo group, and

an aralkyl group having an aryl group selected from phenyl, biphenyl, and naphthyl; or

an aryl, aralkyl, or 5- or 6-membered heterocyclic group with 1-3 nitrogen, oxygen or sulfur atoms, each of which may have 1-4 substituents selected from

a straight chain or branched C₁₋₁₀ alkyl group,

a straight chain or branched C₂₋₁₀ alkenyl group,

a halogen atom selected from fluorine, chlorine, and bromine,

a nitro group, and

a halo-C₁₋₄ lower alkyl group having a halogen atom selected from fluorine, chlorine, and bromine;

Z represents

a hydrogen atom;

a mercapto-protecting group selected from

a C₁₋₄ lower alkoxymethyl group,

a C₁₋₄ lower alkylthiomethyl group,

an aralkyloxymethyl group having an aryl group selected from phenyl, biphenyl, and naphthyl,

an aralkylthiomethyl group having an aryl group selected from phenyl, biphenyl, and naphthyl,

a C₃₋₁₀ cycloalkyloxymethyl group,

a C₅₋₈ cycloalkenyloxymethyl group,

a C₃₋₁₀cycloalkyl C₁₋₁₀ alkoxymethyl group,

an aryloxymethyl group having an aryl group selected from phenyl, biphenyl, and naphthyl,

an arylthiomethyl group having an aryl group selected from phenyl, biphenyl, and naphthyl,

an acyl group,

an acyloxy group,

an aminocarbonyloxymethyl group,

a thiocarbonyl group, and

a thio group.

In some embodiments, the antiviral compound is a compound of Formula (I-1):

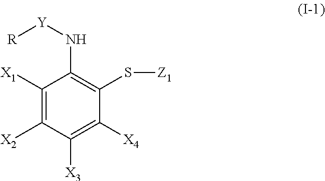

or a pharmaceutically acceptable salt, hydrate, or solvate thereof,
wherein:
R, X₁, X₂, X₃, X₄, and Y are the same as in the above (2) and
Z₁ represents

a hydrogen atom;

a group represented by the formula:

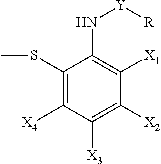

wherein R, X₁, X₂, X₃, X₄, and Y are the same as described above;
—Y₁R₁,
wherein
Y₁ represents

### —CO—; or

### —CS—, and

R₁ represents

a substituted or unsubstituted straight chain or branched C₁₋₁₀ alkyl group;

a C₁₋₄ lower alkoxy group;

a C₁₋₄ lower alkylthio group;

a substituted or unsubstituted amino group;

a substituted or unsubstituted ureido group;

a substituted or unsubstituted C₃₋₁₀ cycloalkyl group;

a substituted or unsubstituted C₃₋₁₀ cycloalkyl C₁₋₁₀ alkyl group;

a substituted or unsubstituted aryl group;

a substituted or unsubstituted aralkyl group;

a substituted or unsubstituted arylalkenyl group;

a substituted or unsubstituted arylthio group;

a substituted or unsubstituted 5- or 6-membered heterocyclic group having 1-3 nitrogen, oxygen, or sulfur atoms; or

a substituted or unsubstituted 5- or 6-membered heteroarylalkyl group; or —S—R₂,

wherein R₂ represents

a substituted or unsubstituted C₁₋₄ lower alkyl group; or

a substituted or unsubstituted aryl group.

In some embodiments, the antiviral compound is a compound of Formula (I-1), or a pharmaceutically acceptable salt, hydrate, or solvate thereof, wherein:

R₁ represents

a straight chain or branched C₁₋₁₀ alkyl group which may have 1-3 substituents selected from

a halogen atom selected from fluorine, chlorine, and bromine,

a C₁₋₄ lower alkoxy group,

an amino group that may be substituted with a C₁₋₄ lower alkyl, acyl, or hydroxyl group,

a C₁₋₄ lower alkylthio group,

a carbamoyl group,

a hydroxyl group,

an acyl group,

an acyloxy group having an acyl group,

a carboxyl group, and

an aryloxy group that may be substituted with a halogen atom selected from fluorine, chlorine, and bromine;

a C₁₋₄ lower alkoxy group;

a C₁₋₄ lower alkylthio group;

an amino or ureido group that may have 1-2 substituents selected from

a C₁₋₄ lower alkyl group,

a hydroxyl group,

an acyl group, and

an aryl group that may be substituted with a lower C₁₋₄ alkoxy group;

a C₃₋₁₀ cycloalkyl or C₃₋₁₀ cycloalkyl C₁₋₁₀ alkyl group that may have substituents selected from

a straight or branched C₁₋₁₀ alkyl group,

a C₃₋₁₀ cycloalkyl group,

a C₅₋₈ cycloalkenyl group,

an aryl group,

an amino group,

a C₁₋₄ lower alkylamino group having a C₁₋₄ lower alkyl group, and

an acylamino group having an acyl group;

an aryl group, an aralkyl group, an arylalkenyl group, or an arylthio group, each of which may have 1-4 substituents selected from

a C₁₋₁₀ alkyl group,

a halogen atom selected from fluorine, chlorine, and bromine,

a nitro group,

a hydroxyl group,

a C₁₋₄ lower alkoxy group,

a C₁₋₄ lower alkylthio group,

an acyl group,

a halo-C₁₋₄ lower alkyl group having a halogen atom selected from fluorine, chlorine, and bromine, and

an amino group that may be substituted with a C₁₋₄ lower alkyl or acyl group;

a 5- or 6-membered heterocyclic group having 1-3 nitrogen, oxygen or sulfur atoms or a 5- or 6-membered heteroarylalkyl group that may have 1-4 substituents selected from

a straight chain or branched C₁₋₁₀ alkyl group,

a halogen atom selected from fluorine, chlorine, and bromine,

an acyl group,

an oxo group, and

an halo-C₁₋₄ lower alkyl group having a halogen atom selected from fluorine, chlorine, and bromine; and

R₂ represents

a C₁₋₄ lower alkyl group that may have 1-3 substituents selected from

a C₁₋₄ lower alkoxy groups,

an amino group that may be substituted with a C₁₋₄ lower alkyl or acyl group,

a C₁₋₄ lower alkylthio group,

a carbamoyl group,

a hydroxyl group,

a carboxyl group,

an acyl group, and

a 5- or 6-membered heterocyclic group having 1-3 nitrogen, oxygen, or sulfur atoms; or

an aryl group that may have 1-4 substituents selected from

a C₁₋₄ lower alkyl group,

a halogen atom selected from fluorine, chlorine, and bromine,

a nitro group,

a hydroxyl group,

a C₁₋₄ lower alkoxy group,

a C₁₋₄ lower alkylthio group,

an acyl group,

an amino group that may be substituted with a C₁₋₄ lower alkyl or acyl group, and

a halo-C₁₋₄ lower alkyl group having a halogen atom selected from fluorine, chlorine, and bromine.

In some embodiments, the antiviral compound is a compound of Formula (I) and is:
bis-[2-(pivaloylamino)phenyl]disulfide;
bis-[2-(2-propylpentanoylamino)phenyl]disulfide;
bis-[2-(1-methylcyclohexanecarbonylamino)phenyl]disulfide;
bis-[2-(1-isopentylcyclopentanecarbonylamino)phenyl]disulfide;
bis-[2-(1-isopentylcyclohexanecarbonylamino)phenyl]disulfide;
N-(2-mercaptophenyl)-2,2-dimethylpropionamide;
N-(2-mercaptophenyl)-1-isopentylcyclohexanecarboxamide;
N-(2-mercaptophenyl)-1-methylcyclohexanecarboxamide;
N-(2-mercaptophenyl)-1-isopentylcyclopentanecarboxamide;
N-(2-mercaptophenyl)-1-isopropylcyclohexanecarboxamide;
N-(4,5-dichloro-2-mercaptophenyl)-1-isopentylcyclohexanecarboxamide;
N-(4,5-dichloro-2-mercaptophenyl)-1-isopentylcyclopentanecarboxamide;
N-(2-mercapto-5-methylphenyl)-1-isopentylcyclohexanecarboxamide;
N-(2-mercapto-4-methylphenyl)-1-isopentylcyclohexanecarboxamide;
S-[2-(1-isopentylcyclohexanecarbonylamino)phenyl]thioacetate;
S-[2-(1-methylcyclohexanecarbonylamino)phenyl]2,2-dimethylthiopropionate;
S-[2-(pivaloylamino)phenyl]phenylthioacetate;
S-[2-(1-isopentylcyclohexanecarbonylamino)phenyl]2,2-dimethylthiopropionate;
S-[2-(1-isopentylcyclohexanecarbonylamino)phenyl]2-acetylamino-3-phenylthiopropionate;
S-[2-(1-isopentylcyclohexanecarbonylamino)phenyl]3-pyridinethiocarboxylate;
S-[2-(1-isopentylcyclohexanecarbonylamino)phenyl]chlorothioacetate;
S-[2-(1-isopentylcyclohexanecarbonylamino)phenyl]methoxythioacetate;
S-[2-(1-isopentylcyclohexanecarbonylamino)phenyl]thiopropionate;
S-[2-(1-isopentylcyclohexanecarbonylamino)phenyl]phenoxythioacetate;
S-[2-(1-isopentylcyclohexanecarbonylamino)phenyl]2-methylthiopropionate;
S-[2-(1-isopentylcyclohexanecarbonylamino)phenyl]4-chlorophenoxythioacetate;
S-[2-(1-isopentylcyclohexanecarbonylamino)phenyl]cyclopropanethiocarboxylate;
S-[2-(1-isopentylcyclohexanecarbonylamino)phenyl]2-acetylamino-4-carbamoylthiobutyrate;
S-[2-(1-isopentylcyclohexanecarbonylamino)phenyl]2-hydroxy-2-methylthiopropionate;
S-[2-(1-isopentylcyclopentanecarbonylamino)phenyl]2,2-dimethylthiopropionate;
S-[2-(1-isopentylcyclopentanecarbonylamino)phenyl]thioacetate;
S-[4,5-dichloro-2-(1-isopentylcyclohexanecarbonylamino)phenyl]2,2-dimethylthiopropionate;
S-[4,5-dichloro-2-(1-isopentylcyclopentanecarbonylamino)phenyl]2,2-dimethylthiopropionate;
S-[2-(1-isopentylcyclohexanecarbonylamino)-4-trifluoromethylphenyl]2,2-dimethylthiopropionate;
O-methyl S-[2-(1-isopentylcyclohexanecarbonylamino)phenyl monothiocarbonate;
S-[2-(1-methylcyclohexanecarbonylamino)phenyl]S-phenyl dithiocarbonate;
S-[2-(1-isopentylcyclohexanecarbonylamino)phenyl]N-phenylthiocarbamate;
S-[2-(pivaloylamino)-4-trifluoromethylphenyl]2,2-dimethylthiopropionate;
S-[4,5-dichloro-2-(1-cyclopropylcyclohexanecarbonylamino)phenyl]2,2-dimethylthiopropionate;
S-[4,5-dichloro-2-(2-cyclohexylpropionylamino)phenyl]2,2-dimethylthiopropionate;
S-[4,5-dichloro-2-(1-pentylcyclohexanecarbonylamino)phenyl]2,2-dimethylthiopropionate;
S-[4,5-dichloro-2-(1-cyclopropylmethylcyclohexanecarbonylamino)phenyl]2,2-dimethylthiopropionate;
S-[4,5-dichloro-2-(1-cyclohexylmethylcyclohexanecarbonylamino)phenyl]2,2-dimethylthiopropionate;
S-[4,5-dichloro-2-(1-isopropylcyclohexanecarbonylamino)phenyl]2,2-dimethylthiopropionate;
S-[4,5-dichloro-2-(1-isopentylcycloheptanecarbonylamino)phenyl]2,2-dimethylthiopropionate;
S-[4,5-dichloro-2-(1-isopentylcyclobutanecarbonylamino)phenyl]2,2-dimethylthiopropionate;
S-[2-(1-isopentylcyclohexanecarbonylamino)-4-nitrophenyl]2,2-dimethylthiopropionate;
S-[4-cyano-2-(1-isopentylcyclohexanecarbonylamino)phenyl]2,2-dimethylthiopropionate;
S-[4-chloro-2-(1-isopentylcyclohexanecarbonylamino)phenyl]2,2-dimethylthiopropionate;
S-[5-chloro-2-(1-isopentylcyclohexanecarbonylamino)phenyl]2,2-dimethylthiopropionate;
S-[4-fluoro-2-(1-isopentylcyclohexanecarbonylamino)phenyl]2,2-dimethylthiopropionate;
S-[4,5-difluoro-2-(1-isopentylcyclohexanecarbonylamino)phenyl]2,2-dimethylthiopropionate;
S-[5-fluoro-2-(1-isopentylcyclohexanecarbonylamino)phenyl]2,2-dimethylthiopropionate;
bis-[4,5-dichloro-2-(1-isopentylcyclohexanecarbonylamino)phenyl]disulfide;
2-tetrahydrofurylmethyl 2-(1-isopentylcyclohexanecarbonyl amino)phenyl disulfide;
N-(2-mercaptophenyl)-1-ethylcyclohexanecarboxamide;
N-(2-mercaptophenyl)-1-propylcyclohexanecarboxamide;
N-(2-mercaptophenyl)-1-butylcyclohexanecarboxamide;
N-(2-mercaptophenyl)-1-isobutylcyclohexanecarboxamide;
S-[2-(1-isopentylcyclohexanecarbonylamino)phenyl]cyclohexanethiocarboxylate;
S-[2-(1-isopentylcyclohexanecarbonylamino)phenyl]thiobenzoate;
S-[2-(1-isopentylcyclohexanecarbonylamino)phenyl]5-carboxythiopentanoate;
S-[2-(1-isopentylcyclohexanecarbonylamino)-4-methylphenyl]thioacetate;
bis-[2-[1-(2-ethylbutyl)cyclohexanecarbonylamino]phenyl]disulfide;
N-(2-mercaptophenyl)-1-(2-ethylbutyl)cyclohexanecarboxamide;
S-[2-[1-(2-ethylbutyl)cyclohexanecarbonylamino]phenyl]2-methylthiopropionate;
S-[2-(1-isobutylcyclohexanecarbonylamino)phenyl]2-methylthiopropionate;
S-[2-[1-(2-ethylbutyl)cyclohexanecarbonylamino]phenyl]1-acetylpiperidine-4-thiocarboxylate;
S-[2-[1-(2-ethylbutyl)cyclohexanecarbonylamino]phenyl]thioacetate;
S-[2-[1-(2-ethylbutyl)cyclohexanecarbonylamino]phenyl]2,2-dimethylthiopropionate;
S-[2-[1-(2-ethylbutyl)cyclohexanecarbonylamino]phenyl]methoxythioacetate;
S-[2-[1-(2-ethylbutyl)cyclohexanecarbonylamino]phenyl]2-hydroxy-2-methylthiopropionate;
S-[2-[1-(2-ethylbutyl)cyclohexanecarbonylamino]phenyl]4-chlorophenoxythioacetate;
S-[2-(1-isobutylcyclohexanecarbonylamino)phenyl]4-chlorophenoxythioacetate; or
S-[2-(1-isobutylcyclohexanecarbonylamino)phenyl]1-acetylpiperidine-4-thiocarboxylate,
   or
   a pharmaceutically acceptable salt, hydrate, or solvate thereof.

In some embodiments, the antiviral compound is a compound of Formula (I-2):

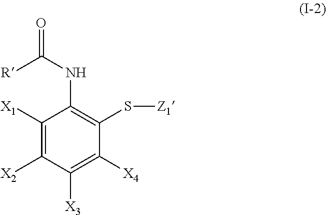

or a pharmaceutically acceptable salt, hydrate, or solvate thereof,
wherein:
R′ represents
a substituted or unsubstituted C₃₋₁₀ cycloalkyl group or
a substituted or unsubstituted C₅₋₈ cycloalkenyl group;
X₁, X₂, X₃, and X₄ are as defined above in Formula (I); and
Z₁′ represents

a hydrogen atom;

a group represented by the formula:

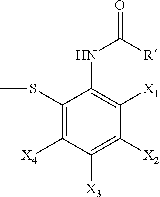

wherein R′, X₁, X₂, X₃, and X₄ are as described above;
—Y₁R₁,
wherein Y₁ and R₁ are the same as defined in Formula (I-1) or

### —S—R₂,

wherein R₂ is the same as defined in Formula (I-1).

In some embodiments, the antiviral compound is a compound of Formula (I-3):

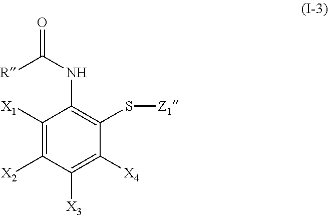

or a pharmaceutically acceptable salt, hydrate, or solvate thereof, wherein:
R″ represents

a 1-substituted-C₃₋₁₀ cycloalkyl group or

a 1-substituted-C₅₋₈ cycloalkenyl group;

X₁, X₂, X₃, and X₄ are the same as defined in Formula (I); and
Z₁″ represents

a hydrogen atom;

a group represented by the formula:

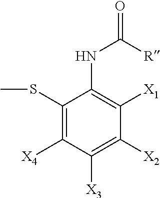

wherein R″, X₁, X₂, X₃, and X₄ are as described above;
—Y₁R₁,
wherein Y₁ and R₁ are the same as defined in Formula (I-1); or

### —S—R₂,

wherein R₂ is the same defined in Formula (I-1).

In some embodiments, the antiviral compound is a compound of Formula (II):

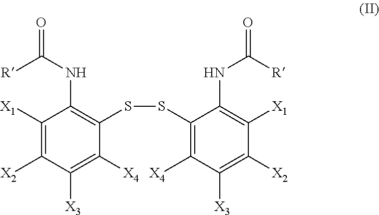

or a pharmaceutically acceptable salt, hydrate, or solvate thereof,
wherein R′, X₁, X₂, X₃, and X₄ are the same as defined in Formula (I-2).

In some embodiments, the antiviral compound is a compound of Formula (II-1):

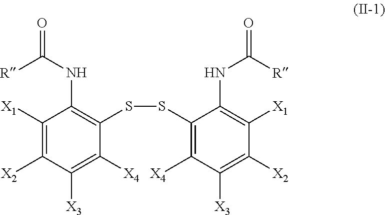

or a pharmaceutically acceptable salt, hydrate, or solvate thereof,
wherein R″, X₁, X₂, X₃, and X₄ are the same as in the above (9).

In some embodiments, the antiviral compound is a compound of Formula (III):

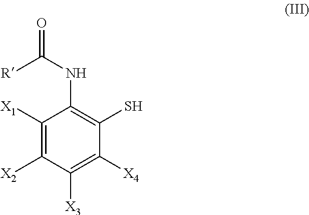

or
a pharmaceutically acceptable salt, hydrate, or solvate thereof,
wherein R′, X₁, X₂, X₃, and X₄ are the same as defined in Formula (I-2).

In some embodiments, the antiviral compound is a compound of Formula (III-1):

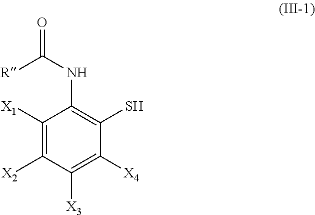

or a pharmaceutically acceptable salt, hydrate, or solvate thereof,
wherein R″, X₁, X₂, X₃, and X₄ are the same as defined in Formula (I-3).

In some embodiments, the antiviral compound is a compound of Formula (IV):

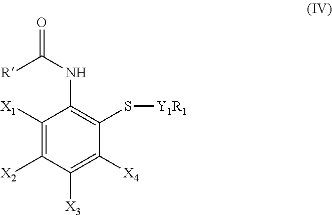

or a pharmaceutically acceptable salt, hydrate, or solvate thereof,
wherein R′, X₁, X₂, X₃, X₄, Y₁, and R₁ are the same as defined in Formula (I-2).

In some embodiments, the antiviral compound is a compound of Formula (IV-1):

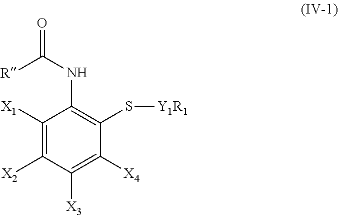

or a pharmaceutically acceptable salt, hydrate, or solvate thereof,
wherein R″, X₁, X₂, X₃, X₄, Y₁, and R₁ are the same as defined in Formula (I-3).

In some embodiments, the antiviral compound is a compound of Formula (V):

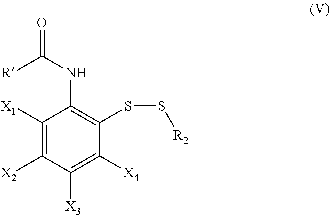

or a pharmaceutically acceptable salt, hydrate, or solvate thereof, wherein R′, X₁, X₂, X₃, X₄, and R₂ are the same as defined in Formula (I-2).

In some embodiments, the antiviral compound is a compound of Formula (V-1):

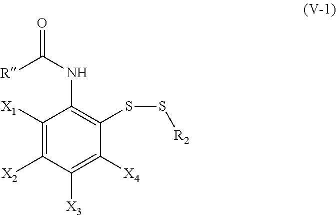

or a pharmaceutically acceptable salt, hydrate, or solvate thereof,
wherein R″, X₁, X₂, X₃, X₄, and R₂ are the same as defined in Formula (I-3).

In some embodiments, the antiviral compound is a compound of Formula (VI):

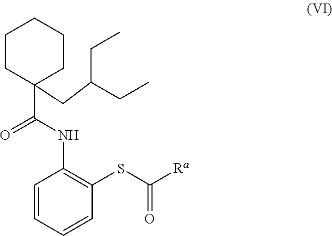

or a pharmaceutically acceptable salt, hydrate, or solvate thereof, wherein R^{a} represents:

a straight chain or branched C₁₋₁₀ alkyl group;

a straight chain or branched C₂₋₁₀ alkenyl group;

a straight chain or branched C₂₋₁₀ alkynyl group;

a halo-C₁₋₄ lower alkyl group;

a substituted or unsubstituted C₃₋₁₀ cycloalkyl group;

a substituted or unsubstituted C₅₋₈ cycloalkenyl group; or

a substituted or unsubstituted C₃₋₁₀ cycloalkyl C₁₋₄ alkyl group.

In some embodiments, R^{a} represents:

a straight chain or branched C₁₋₁₀ alkyl group;

a straight chain or branched C₂₋₁₀ alkenyl group;

a straight chain or branched C₂₋₁₀ alkynyl group;

a substituted or unsubstituted C₃₋₁₀ cycloalkyl group; or

a substituted or unsubstituted C₅₋₈ cycloalkenyl group.

In some embodiments of Formula (VI), R^{a} is a straight chain or branched C₁₋₅ alkyl group. In some embodiments, the alkyl group is a methyl, ethyl, propyl, isopropyl, butyl, t-butyl, pentyl, isoamyl, or neopentyl group.

In some embodiments of Formula (VI), R^{a} is a straight chain or branched C₂₋₆ alkenyl group. In some embodiments, the alkenyl group is an ethenyl, propenyl, butenyl (e.g., 1-butenyl or 2-butenyl), pentenyl (e.g., 1-pentenyl, 2-pentenyl, or 3-pentenyl), or hexenyl (e.g., 1-hexenyl, 2-hexenyl, 3-hexenyl, or 4-hexenyl) group.

In some embodiments of Formula (VI), R^{a} is a straight chain or branched C₂₋₆ alkynyl group. In some embodiments, the alkynyl group is an ethynyl, propynyl, butynyl (e.g., 1-butynyl or 2-butynyl), pentynyl (e.g., 1-pentynyl, 2-pentynyl, or 3-pentynyl), or hexynyl (e.g., 1-hexynyl, 2-hexynyl, 3-hexynyl, or 4-hexynyl) group.

In some embodiments of Formula (VI), R^{a} is a substituted or unsubstituted C₃₋₈ cycloalkyl group. In some embodiments, the cycloalkyl group is a cyclopropyl, cyclobutyl, cyclopentyl, cyclohexyl, or cycloheptyl group.

In some embodiments of Formula (VI), R^{a} is a substituted or unsubstituted C₆₋₇ cycloalkenyl group. In some embodiments, the cycloalkenyl is a cyclohexenyl group.

In some embodiments, the antiviral compound is a compound of Formula (VI) and has any one of the structures as set forth below:

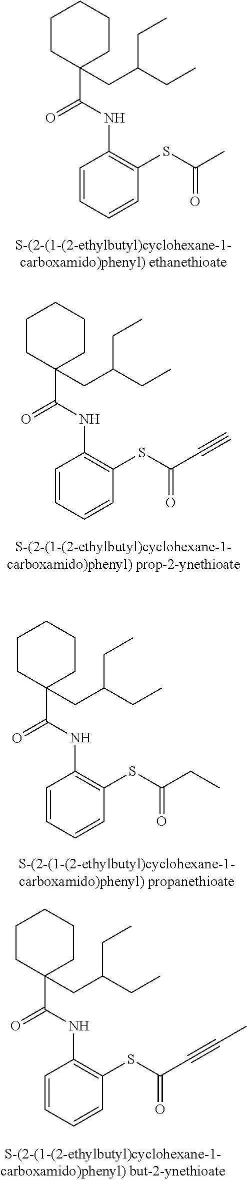 [SMILES: CCC(CC)CC1(C(=O)Nc2ccccc2SC(C)=O)CCCCC1 ; C#CC(=O)Sc1ccccc1NC(=O)C1(CC(CC)CC)CCCCC1 ; CCC(=O)Sc1ccccc1NC(=O)C1(CC(CC)CC)CCCCC1 ; CC#CC(=O)Sc1ccccc1NC(=O)C1(CC(CC)CC)CCCCC1]

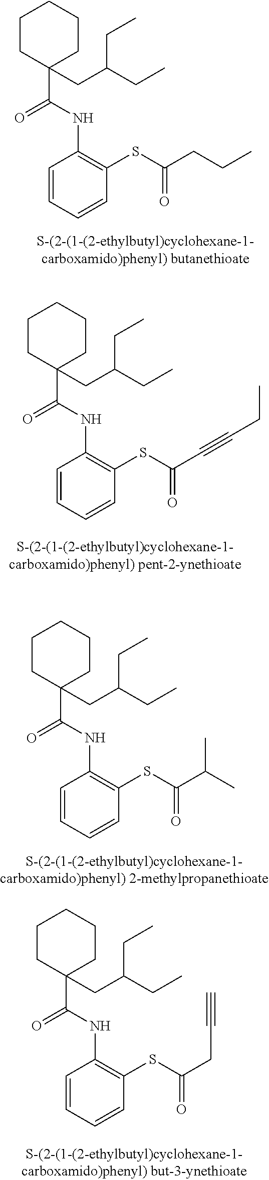 [SMILES: CCCC(=O)Sc1ccccc1NC(=O)C1(CC(CC)CC)CCCCC1 ; CCC#CC(=O)Sc1ccccc1NC(=O)C1(CC(CC)CC)CCCCC1 ; CCC(CC)CC1(C(=O)Nc2ccccc2SC(=O)C(C)C)CCCCC1 ; C#CCC(=O)Sc1ccccc1NC(=O)C1(CC(CC)CC)CCCCC1]

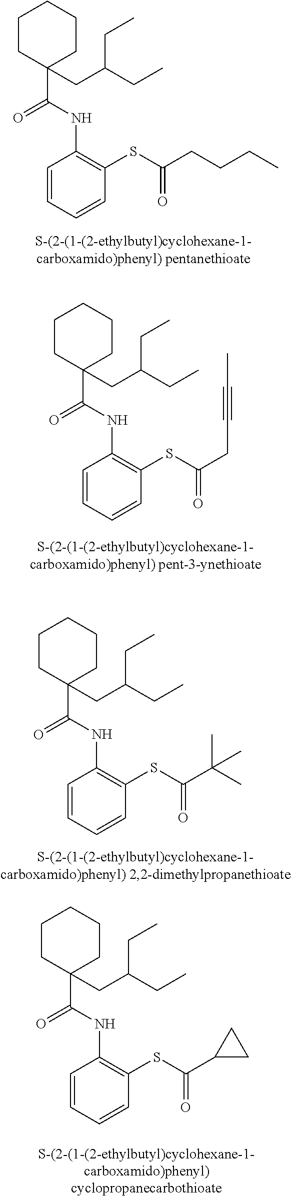 [SMILES: CCCCC(=O)Sc1ccccc1NC(=O)C1(CC(CC)CC)CCCCC1 ; CC#CCC(=O)Sc1ccccc1NC(=O)C1(CC(CC)CC)CCCCC1 ; CCC(CC)CC1(C(=O)Nc2ccccc2SC(=O)C(C)(C)C)CCCCC1 ; CCC(CC)CC1(C(=O)Nc2ccccc2SC(=O)C2CC2)CCCCC1]

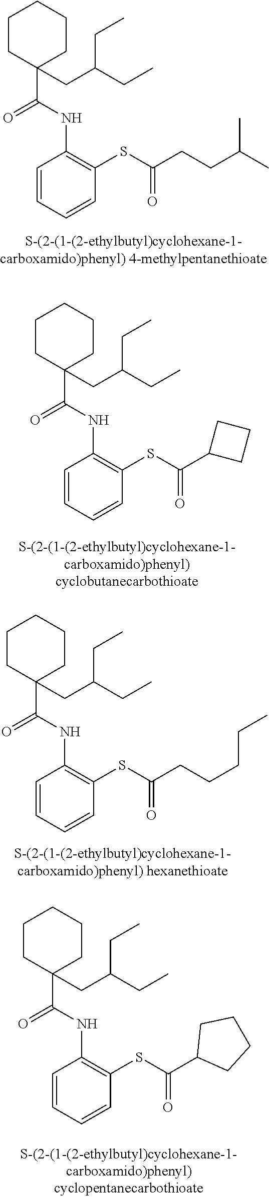 [SMILES: CCC(CC)CC1(C(=O)Nc2ccccc2SC(=O)CCC(C)C)CCCCC1 ; CCC(CC)CC1(C(=O)Nc2ccccc2SC(=O)C2CCC2)CCCCC1 ; CCCCCC(=O)Sc1ccccc1NC(=O)C1(CC(CC)CC)CCCCC1 ; CCC(CC)CC1(C(=O)Nc2ccccc2SC(=O)C2CCCC2)CCCCC1]

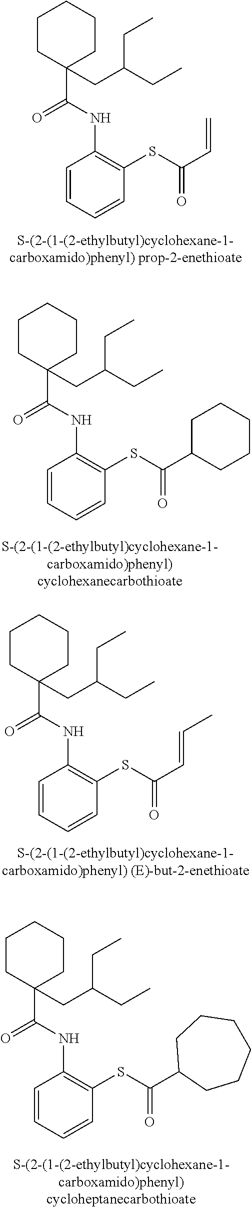 [SMILES: C=CC(=O)Sc1ccccc1NC(=O)C1(CC(CC)CC)CCCCC1 ; CCC(CC)CC1(C(=O)Nc2ccccc2SC(=O)C2CCCCC2)CCCCC1 ; C/C=C/C(=O)Sc1ccccc1NC(=O)C1(CC(CC)CC)CCCCC1 ; CCC(CC)CC1(C(=O)Nc2ccccc2SC(=O)C2CCCCCC2)CCCCC1]

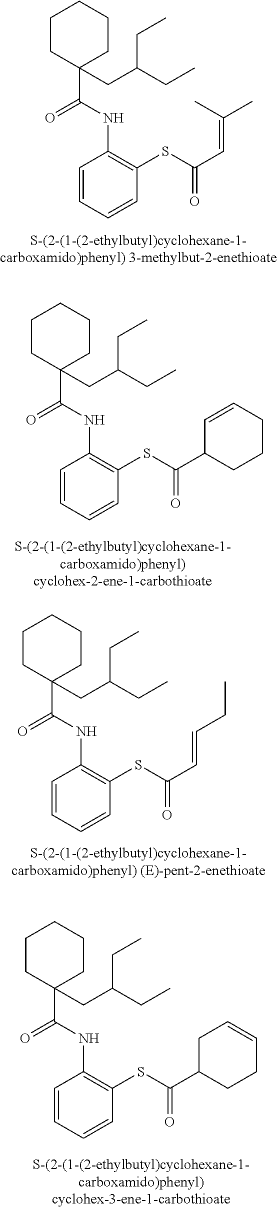 [SMILES: CCC(CC)CC1(C(=O)Nc2ccccc2SC(=O)C=C(C)C)CCCCC1 ; CCC(CC)CC1(C(=O)Nc2ccccc2SC(=O)C2C=CCCC2)CCCCC1 ; CC/C=C/C(=O)Sc1ccccc1NC(=O)C1(CC(CC)CC)CCCCC1 ; CCC(CC)CC1(C(=O)Nc2ccccc2SC(=O)C2CC=CCC2)CCCCC1]

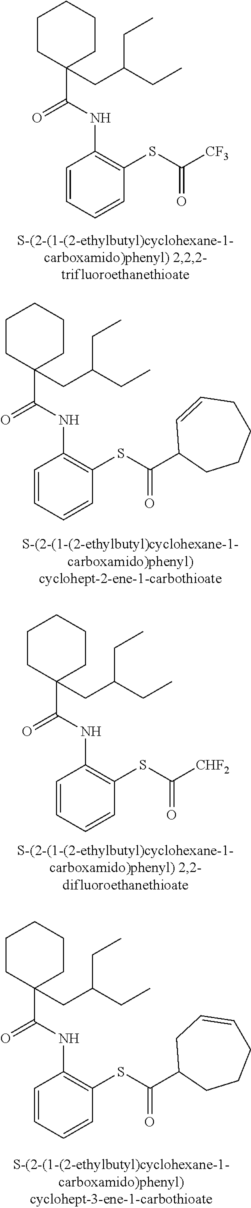 [SMILES: CCC(CC)CC1(C(=O)Nc2ccccc2SC(=O)C(F)(F)F)CCCCC1 ; CCC(CC)CC1(C(=O)Nc2ccccc2SC(=O)C2C=CCCCC2)CCCCC1 ; CCC(CC)CC1(C(=O)Nc2ccccc2SC(=O)C(F)F)CCCCC1 ; CCC(CC)CC1(C(=O)Nc2ccccc2SC(=O)C2CC=CCCC2)CCCCC1]

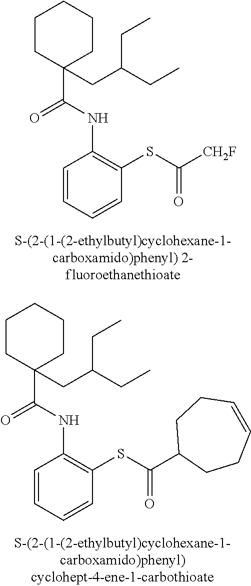

or a pharmaceutically acceptable salt, hydrate, or solvate thereof. The structure's chemical name appears to the structure's immediate right.

In some embodiments, the antiviral compound is a compound of Formula (VII):

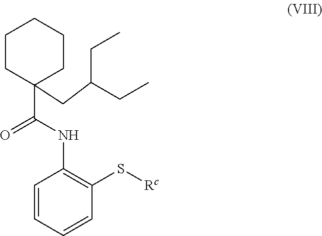

or a pharmaceutically acceptable salt, hydrate, or solvate thereof, wherein: R^{c} represents:

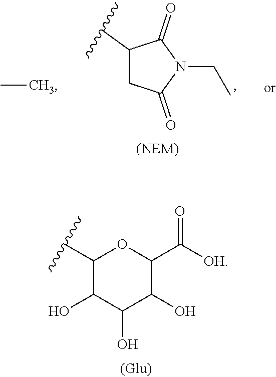

In some embodiments, the antiviral compound is a compound of Formula (I-2) and is:
bis-[2-(1-methylcyclohexanecarbonylamino)phenyl]disulfide;
bis-[2-(1-isopentylcyclopentanecarbonylamino)phenyl]disulfide;
bis-[2-(1-isopentylcyclohexanecarbonylamino)phenyl]disulfide;
N-(2-mercaptophenyl)-1-isopentylcyclohexanecarboxamide;
N-(2-mercaptophenyl)-1-methylcyclohexanecarboxamide;
N-(2-mercaptophenyl)-1-isopentylcyclopentanecarboxamide;
N-(2-mercaptophenyl)-1-isopropylcyclohexanecarboxamide;
N-(4,5-dichloro-2-mercaptophenyl)-1-isopentylcyclohexanecarboxamide;
N-(4,5-dichloro-2-mercaptophenyl)-1-isopentylcyclopentanecarboxamide;
N-(2-mercapto-5-methylphenyl)-1-isopentylcyclohexanecarboxamide;
N-(2-mercapto-4-methylphenyl)-1-isopentylcyclohexanecarboxamide;
S-[2-(1-isopentylcyclohexanecarbonylamino)phenyl]thioacetate;
S-[2-(1-methylcyclohexanecarbonylamino)phenyl]2,2-dimethylthiopropionate;
S-[2-(1-isopentylcyclohexanecarbonylamino)phenyl]2,2-dimethylthiopropionate;
S-[2-(1-isopentylcyclohexanecarbonylamino)phenyl]2-acetylamino-3-phenylthiopropionate;
S-[2-(1-isopentylcyclohexanecarbonylamino)phenyl]3-pyridinethiocarboxylate;
S-[2-(1-isopentylcyclohexanecarbonylamino)phenyl]chlorothioacetate;
S-[2-(1-isopentylcyclohexanecarbonylamino)phenyl]methoxythioacetate;
S-[2-(1-isopentylcyclohexanecarbonylamino)phenyl]thiopropionate;
S-[2-(1-isopentylcyclohexanecarbonylamino)phenyl]phenoxythioacetate;
S-[2-(1-isopentylcyclohexanecarbonylamino)phenyl]2-methylthiopropionate;
S-[2-(1-isopentylcyclohexanecarbonylamino)phenyl]4-chlorophenoxythioacetate;
S-[2-(1-isopentylcyclohexanecarbonylamino)phenyl]cyclopropanethiocarboxylate;
S-[2-(1-isopentylcyclohexanecarbonylamino)phenyl]2-acetylamino-4-carbamoylthiobutyrate;
S-[2-(1-isopentylcyclohexanecarbonylamino)phenyl]2-hydroxy-2-methylthiopropionate;
S-[2-(1-isopentylcyclopentanecarbonylamino)phenyl]2,2-dimethylpropionate;
S-[2-(1-isopentylcyclopentanecarbonylamino)phenyl]thioacetate;
S-[4,5-dichloro-2-(1-isopentylcyclohexanecarbonylamino)phenyl]2,2-dimethylthiopropionate;
S-[4,5-dichloro-2-(1-isopentylcyclopentanecarbonylamino)phenyl]2,2-dimethylthiopropionate;
S-[2-(1-isopentylcyclohexanecarbonylamino)-4-trifluoromethylphenyl]2,2-dimethylthiopropionate;
O-methyl S-[2-(1-isopentylcyclohexanecarbonylamino)phenyl]monothiocarbonate;
S-[2-(1-methylcyclohexanecarbonylamino)phenyl]S-phenyldithiocarbonate;
S-[2-(1-isopentylcyclohexanecarbonylamino)phenyl]N-phenylthiocarbamate;
S-[4,5-dichloro-2-(1-cyclopropylcyclohexanecarbonylamino)phenyl]2,2-dimethylthiopropionate;
S-[4,5-dichloro-2-(1-pentylcyclohexanecarbonylamino)phenyl]2,2-dimethylthiopropionate;
S-[4,5-dichloro-2-(1-cyclopropylmethylcyclohexanecarbonylamino)phenyl]2,2-dimethylthiopropionate;
S-[4,5-dichloro-2-(1-cyclohexylmethylcyclohexanecarbonylamino)phenyl]2,2-dimethylthiopropioate;
S-[4,5-dichloro-2-(1-isopropylcyclohexanecarbonylamino)phenyl]2,2-dimethylthiopropionate;
S-[4,5-dichloro-2-(1-isopentylcycloheptanecarbonylamino)phenyl]2,2-dimethylthiopropionate;
S-[4,5-dichloro-2-(1-isopentylcyclobutanecarbonylamino)phenyl]2,2-dimethylthiopropionate;
S-[2-(1-isopentylcyclohexanecarbonylamino)-4-nitrophenyl]2,2-dimethylthiopropionate;
S-[4-cyano-2-(1-isopentylcyclohexanecarbonylamino)phenyl]2,2-dimethylthiopropionate;
S-[4-chloro-2-(1-isopentylcyclohexanecarbonylamino)phenyl]2,2-dimethylthiopropionate;
S-[5-chloro-2-(1-isopentylcyclohexanecarbonylamino)phenyl]2,2-dimethylthiopropionate;
S-[4-fluoro-2-(1-isopentylcyclohexanecarbonylamino)phenyl]2,2-dimethylthiopropionate;
S-[4,5-difluoro-2-(1-isopentylcyclohexanecarbonylamino)phenyl]2,2-dimethylthiopropionate;
S-[5-fluoro-2-(1-isopentylcyclohexanecarbonylamino)phenyl]2,2-dimethylthiopropionate;
bis-[4,5-dichloro-2-(1-isopentylcyclohexanecarbonylamino)phenyl]disulfide;
2-tetrahydrofurylmethyl2-(1-isopentylcyclohexanecarbonylamino)phenyl disulfide;
N-(2-mercaptophenyl)-1-ethylcyclohexanecarboxamide;
N-(2-mercaptophenyl)-1-propylcyclohexanecarboxamide;
N-(2-mercaptophenyl)-1-butylcyclohexanecarboxamide;
N-(2-mercaptophenyl)-1-isobutylcyclohexanecarboxamide;
S-[2-(1-isopentylcyclohexanecarbonylamino)phenyl]cyclohexanethiocarboxylate;
S-[2-(1-isopentylcyclohexanecarbonylamino)phenyl]thiobenzoate;
S-[2-(1-isopentylcyclohexanecarbonylamino)phenyl]5-carboxythiopentanoate;
S-[2-(1-isopentylcyclohexanecarbonylamino)-4-methylphenyl]thioacetate;
bis-[2-[1-(2-ethylbutyl)cyclohexanecarbonylamino]phenyl]disulfide;
N-(2-mercaptophenyl)-1-(2-ethylbutyl)cyclohexanecarboxamide;
S-[2-[1-(2-ethylbutyl)cyclohexanecarbonylamino]phenyl]2-methylthiopropionate;
S-[2-[1-isobutylcyclohexanecarbonylamino]phenyl]2-methylthiopropionate;
S-[2-[1-(2-ethylbutyl)cyclohexanecarbonylamino]phenyl]1-acetylpiperidine-4-thiocarboxylate;
S-[2-[1-(2-ethylbutyl)cyclohexanecarbonylamino]phenyl]thioacetate;
S-[2-[1-(2-ethylbutyl)cyclohexanecarbonylamino]phenyl]2,2-dimethylthiopropionate;
S-[2-[1-(2-ethylbutyl)cyclohexanecarbonylamino]phenyl]methoxythioacetate;
S-[2-[1-(2-ethylbutyl)cyclohexanecarbonylamino]phenyl]2-hydroxy-2-methylpropionate;
S-[2-[1-(2-ethylbutyl)cyclohexanecarbonylamino]phenyl]4-chlorophenoxythioacetate;
S-[2-(1-isobutylcyclohexanecarbonylamino)phenyl]4-chlorophenoxythioacetate; or
S-[2-(1-isobutylcyclohexanecarbonylamino)phenyl]1-acetylpiperidine-4-thiocarboxylate,
   or
   a pharmaceutically acceptable salt, hydrate, or solvate thereof.

In some embodiments, the antiviral compound has any one of the structures shown in the following table:

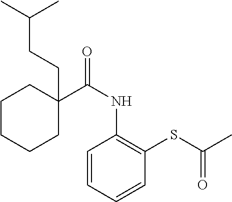 [SMILES: CC(=O)Sc1ccccc1NC(=O)C1(CCC(C)C)CCCCC1]

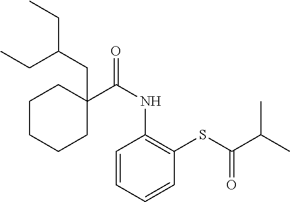 [SMILES: CCC(CC)CC1(C(=O)Nc2ccccc2SC(=O)C(C)C)CCCCC1]

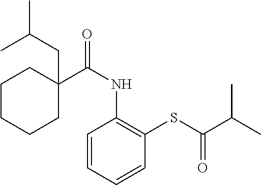 [SMILES: CC(C)CC1(C(=O)Nc2ccccc2SC(=O)C(C)C)CCCCC1]

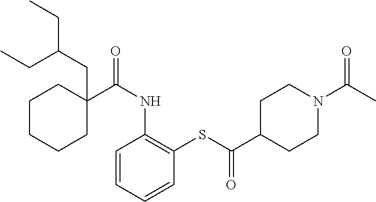 [SMILES: CCC(CC)CC1(C(=O)Nc2ccccc2SC(=O)C2CCN(C(C)=O)CC2)CCCCC1]

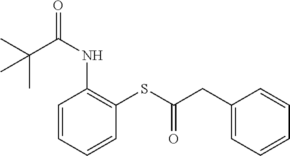 [SMILES: CC(C)(C)C(=O)Nc1ccccc1SC(=O)Cc1ccccc1]

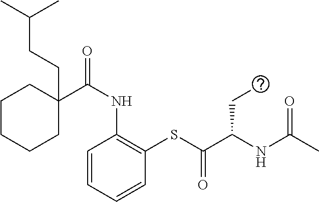 [SMILES: CC[C@H](NC(C)=O)C(=O)Sc1ccccc1NC(=O)C1(CCC(C)C)CCCCC1]

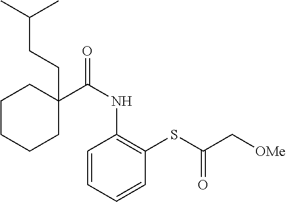 [SMILES: COCC(=O)Sc1ccccc1NC(=O)C1(CCC(C)C)CCCCC1]

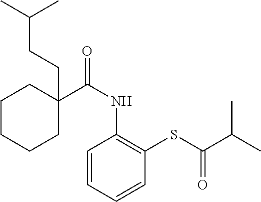 [SMILES: CC(C)CCC1(C(=O)Nc2ccccc2SC(=O)C(C)C)CCCCC1]

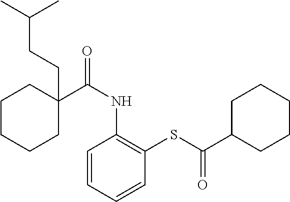 [SMILES: CC(C)CCC1(C(=O)Nc2ccccc2SC(=O)C2CCCCC2)CCCCC1]

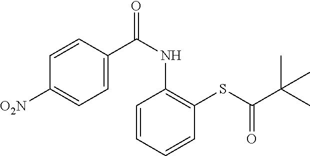 [SMILES: CC(C)(C)C(=O)Sc1ccccc1NC(=O)c1ccc([N+](=O)[O-])cc1]

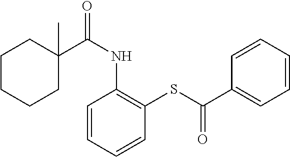 [SMILES: CC1(C(=O)Nc2ccccc2SC(=O)c2ccccc2)CCCCC1]

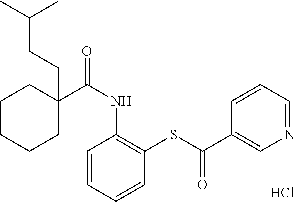 [SMILES: CC(C)CCC1(C(=O)Nc2ccccc2SC(=O)c2cccnc2)CCCCC1 ; Cl]

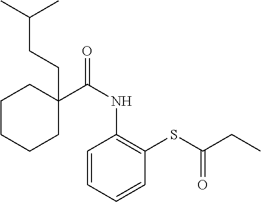 [SMILES: CCC(=O)Sc1ccccc1NC(=O)C1(CCC(C)C)CCCCC1]

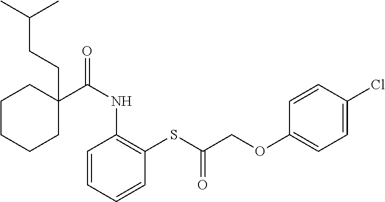 [SMILES: CC(C)CCC1(C(=O)Nc2ccccc2SC(=O)COc2ccc(Cl)cc2)CCCCC1]

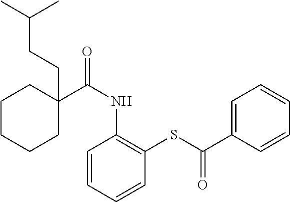 [SMILES: CC(C)CCC1(C(=O)Nc2ccccc2SC(=O)c2ccccc2)CCCCC1]

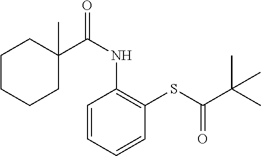 [SMILES: CC(C)(C)C(=O)Sc1ccccc1NC(=O)C1(C)CCCCC1]

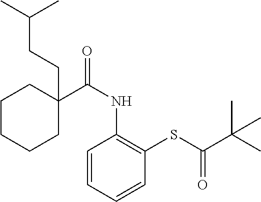 [SMILES: CC(C)CCC1(C(=O)Nc2ccccc2SC(=O)C(C)(C)C)CCCCC1]

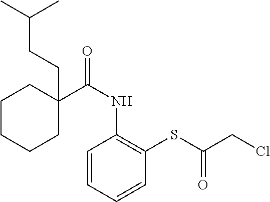 [SMILES: CC(C)CCC1(C(=O)Nc2ccccc2SC(=O)CCl)CCCCC1]

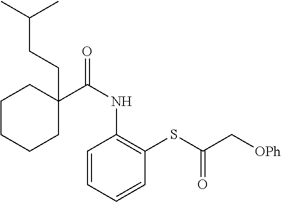 [SMILES: CC(C)CCC1(C(=O)Nc2ccccc2SC(=O)COc2ccccc2)CCCCC1]

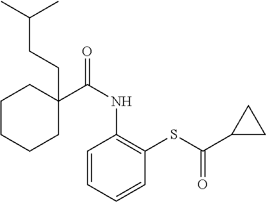 [SMILES: CC(C)CCC1(C(=O)Nc2ccccc2SC(=O)C2CC2)CCCCC1]

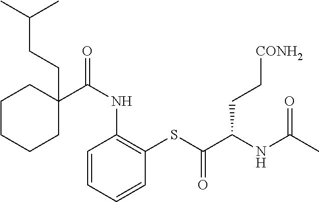 [SMILES: CC(=O)N[C@@H](CCC(N)=O)C(=O)Sc1ccccc1NC(=O)C1(CCC(C)C)CCCCC1]

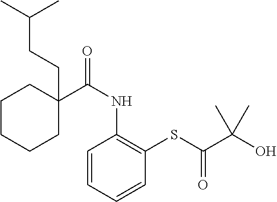 [SMILES: CC(C)CCC1(C(=O)Nc2ccccc2SC(=O)C(C)(C)O)CCCCC1]

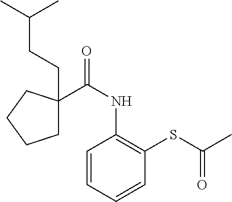 [SMILES: CC(=O)Sc1ccccc1NC(=O)C1(CCC(C)C)CCCC1]

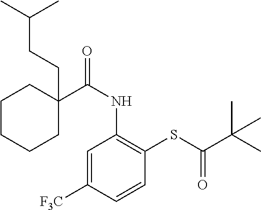 [SMILES: CC(C)CCC1(C(=O)Nc2cc(C(F)(F)F)ccc2SC(=O)C(C)(C)C)CCCCC1]

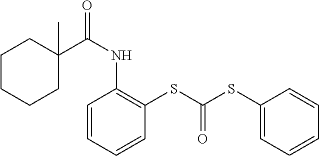 [SMILES: CC1(C(=O)Nc2ccccc2SC(=O)Sc2ccccc2)CCCCC1]

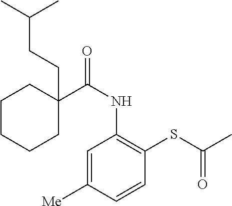 [SMILES: CC(=O)Sc1ccc(C)cc1NC(=O)C1(CCC(C)C)CCCCC1]

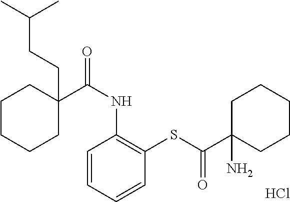 [SMILES: CC(C)CCC1(C(=O)Nc2ccccc2SC(=O)C2(N)CCCCC2)CCCCC1 ; Cl]

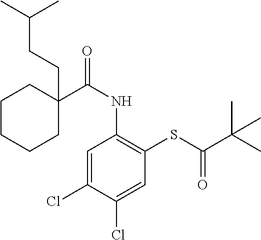 [SMILES: CC(C)CCC1(C(=O)Nc2cc(Cl)c(Cl)cc2SC(=O)C(C)(C)C)CCCCC1]

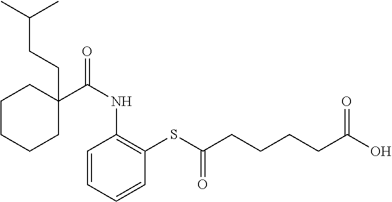 [SMILES: CC(C)CCC1(C(=O)Nc2ccccc2SC(=O)CCCCC(=O)O)CCCCC1]

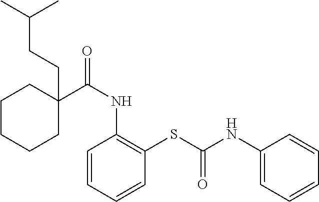 [SMILES: CC(C)CCC1(C(=O)Nc2ccccc2SC(=O)Nc2ccccc2)CCCCC1]

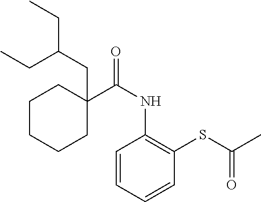 [SMILES: CCC(CC)CC1(C(=O)Nc2ccccc2SC(C)=O)CCCCC1]

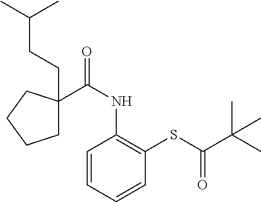 [SMILES: CC(C)CCC1(C(=O)Nc2ccccc2SC(=O)C(C)(C)C)CCCC1]

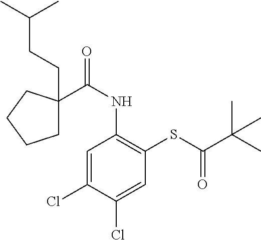 [SMILES: CC(C)CCC1(C(=O)Nc2cc(Cl)c(Cl)cc2SC(=O)C(C)(C)C)CCCC1]

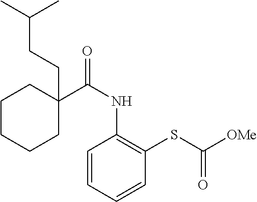 [SMILES: COC(=O)Sc1ccccc1NC(=O)C1(CCC(C)C)CCCCC1]

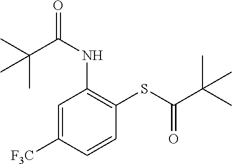 [SMILES: CC(C)(C)C(=O)Nc1cc(C(F)(F)F)ccc1SC(=O)C(C)(C)C]

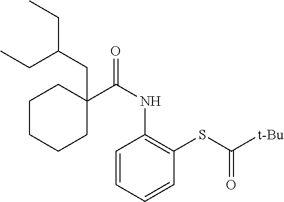 [SMILES: CCC(CC)CC1(C(=O)Nc2ccccc2SC(=O)C(C)(C)C)CCCCC1]

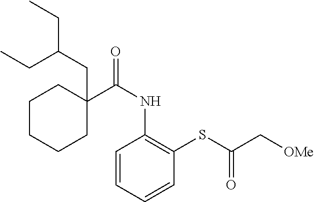 [SMILES: CCC(CC)CC1(C(=O)Nc2ccccc2SC(=O)COC)CCCCC1]

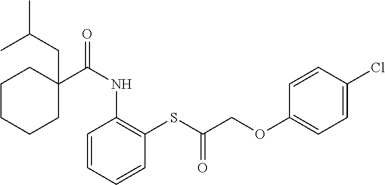 [SMILES: CC(C)CC1(C(=O)Nc2ccccc2SC(=O)COc2ccc(Cl)cc2)CCCCC1]

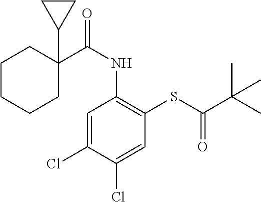 [SMILES: CC(C)(C)C(=O)Sc1cc(Cl)c(Cl)cc1NC(=O)C1(C2CC2)CCCCC1]

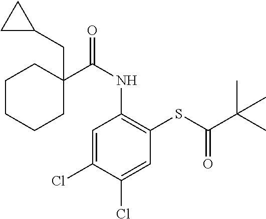 [SMILES: CC(C)(C)C(=O)Sc1cc(Cl)c(Cl)cc1NC(=O)C1(CC2CC2)CCCCC1]

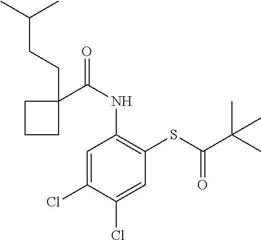 [SMILES: CC(C)CCC1(C(=O)Nc2cc(Cl)c(Cl)cc2SC(=O)C(C)(C)C)CCC1]

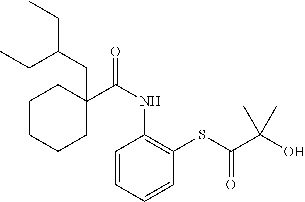 [SMILES: CCC(CC)CC1(C(=O)Nc2ccccc2SC(=O)C(C)(C)O)CCCCC1]

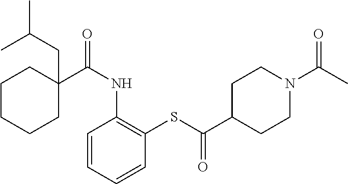 [SMILES: CC(=O)N1CCC(C(=O)Sc2ccccc2NC(=O)C2(CC(C)C)CCCCC2)CC1]

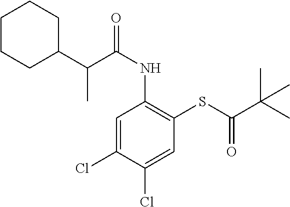 [SMILES: CC(C(=O)Nc1cc(Cl)c(Cl)cc1SC(=O)C(C)(C)C)C1CCCCC1]

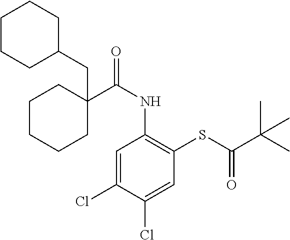 [SMILES: CC(C)(C)C(=O)Sc1cc(Cl)c(Cl)cc1NC(=O)C1(CC2CCCCC2)CCCCC1]

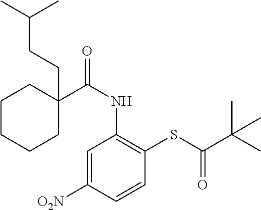 [SMILES: CC(C)CCC1(C(=O)Nc2cc([N+](=O)[O-])ccc2SC(=O)C(C)(C)C)CCCCC1]

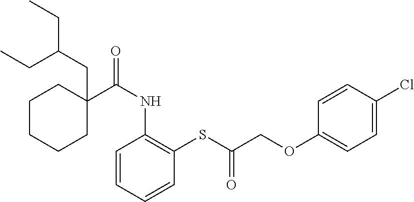 [SMILES: CCC(CC)CC1(C(=O)Nc2ccccc2SC(=O)COc2ccc(Cl)cc2)CCCCC1]

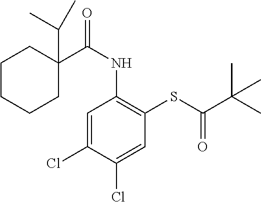 [SMILES: CC(C)C1(C(=O)Nc2cc(Cl)c(Cl)cc2SC(=O)C(C)(C)C)CCCCC1]

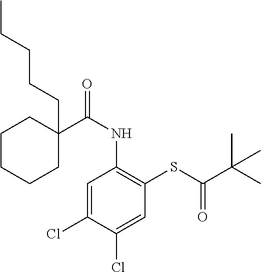 [SMILES: CCCCCC1(C(=O)Nc2cc(Cl)c(Cl)cc2SC(=O)C(C)(C)C)CCCCC1]

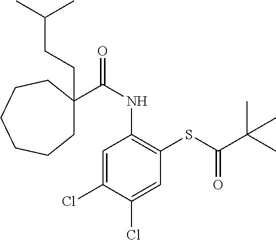 [SMILES: CC(C)CCC1(C(=O)Nc2cc(Cl)c(Cl)cc2SC(=O)C(C)(C)C)CCCCCC1]

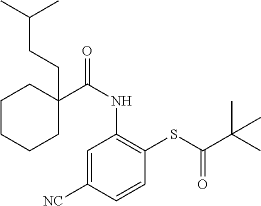 [SMILES: CC(C)CCC1(C(=O)Nc2cc(C#N)ccc2SC(=O)C(C)(C)C)CCCCC1]

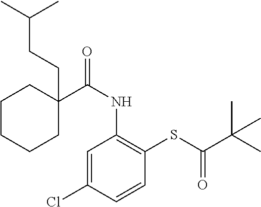 [SMILES: CC(C)CCC1(C(=O)Nc2cc(Cl)ccc2SC(=O)C(C)(C)C)CCCCC1]

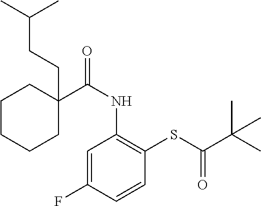 [SMILES: CC(C)CCC1(C(=O)Nc2cc(F)ccc2SC(=O)C(C)(C)C)CCCCC1]

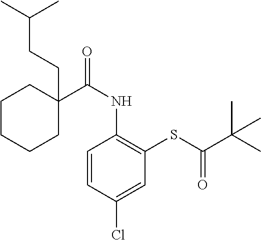 [SMILES: CC(C)CCC1(C(=O)Nc2ccc(Cl)cc2SC(=O)C(C)(C)C)CCCCC1]

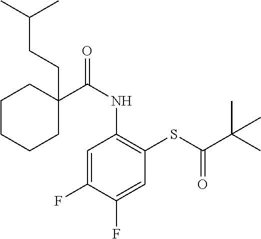 [SMILES: CC(C)CCC1(C(=O)Nc2cc(F)c(F)cc2SC(=O)C(C)(C)C)CCCCC1]

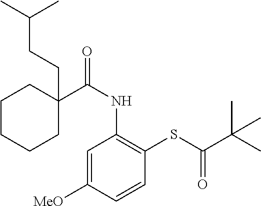 [SMILES: COc1ccc(SC(=O)C(C)(C)C)c(NC(=O)C2(CCC(C)C)CCCCC2)c1]

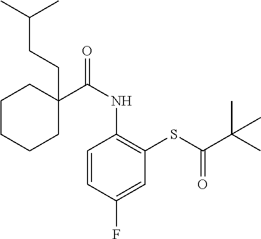 [SMILES: CC(C)CCC1(C(=O)Nc2ccc(F)cc2SC(=O)C(C)(C)C)CCCCC1]

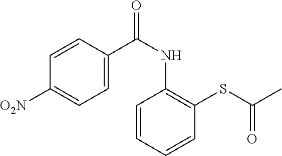 [SMILES: CC(=O)Sc1ccccc1NC(=O)c1ccc([N+](=O)[O-])cc1]

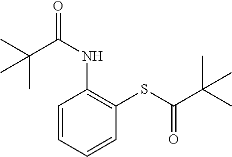 [SMILES: CC(C)(C)C(=O)Nc1ccccc1SC(=O)C(C)(C)C]

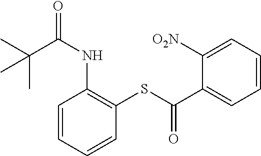 [SMILES: CC(C)(C)C(=O)Nc1ccccc1SC(=O)c1ccccc1[N+](=O)[O-]]

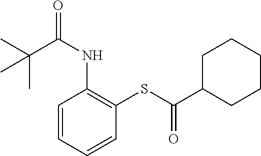 [SMILES: CC(C)(C)C(=O)Nc1ccccc1SC(=O)C1CCCCC1]

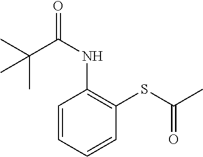 [SMILES: CC(=O)Sc1ccccc1NC(=O)C(C)(C)C]

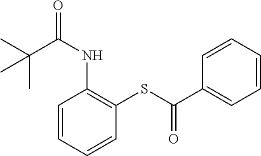 [SMILES: CC(C)(C)C(=O)Nc1ccccc1SC(=O)c1ccccc1]

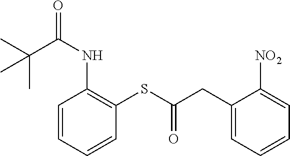 [SMILES: CC(C)(C)C(=O)Nc1ccccc1SC(=O)Cc1ccccc1[N+](=O)[O-]]

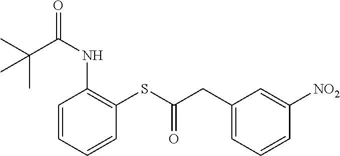 [SMILES: CC(C)(C)C(=O)Nc1ccccc1SC(=O)Cc1cccc([N+](=O)[O-])c1]

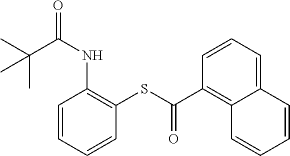 [SMILES: CC(C)(C)C(=O)Nc1ccccc1SC(=O)c1cccc2ccccc12]

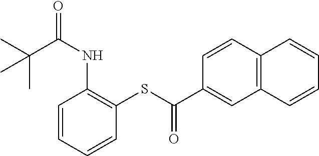 [SMILES: CC(C)(C)C(=O)Nc1ccccc1SC(=O)c1ccc2ccccc2c1]

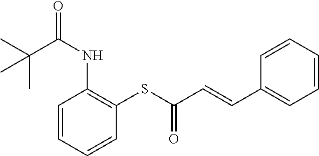 [SMILES: CC(C)(C)C(=O)Nc1ccccc1SC(=O)/C=C/c1ccccc1]

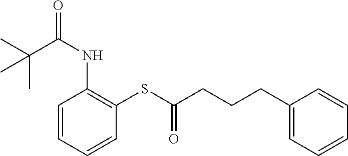 [SMILES: CC(C)(C)C(=O)Nc1ccccc1SC(=O)CCCc1ccccc1]

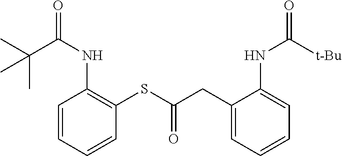 [SMILES: CC(C)(C)C(=O)Nc1ccccc1CC(=O)Sc1ccccc1NC(=O)C(C)(C)C]

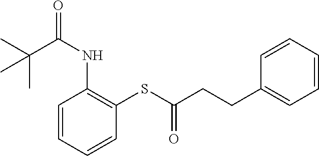 [SMILES: CC(C)(C)C(=O)Nc1ccccc1SC(=O)CCc1ccccc1]

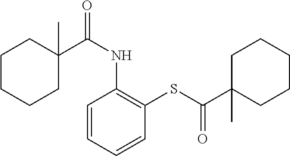 [SMILES: CC1(C(=O)Nc2ccccc2SC(=O)C2(C)CCCCC2)CCCCC1]

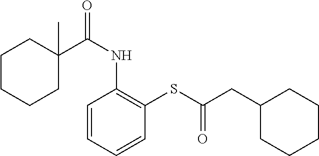 [SMILES: CC1(C(=O)Nc2ccccc2SC(=O)CC2CCCCC2)CCCCC1]

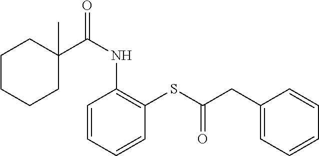 [SMILES: CC1(C(=O)Nc2ccccc2SC(=O)Cc2ccccc2)CCCCC1]

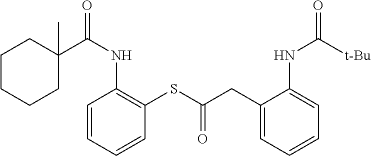 [SMILES: CC(C)(C)C(=O)Nc1ccccc1CC(=O)Sc1ccccc1NC(=O)C1(C)CCCCC1]

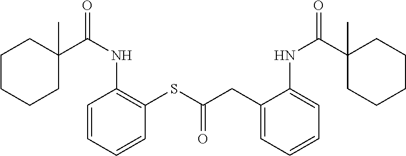 [SMILES: CC1(C(=O)Nc2ccccc2CC(=O)Sc2ccccc2NC(=O)C2(C)CCCCC2)CCCCC1]

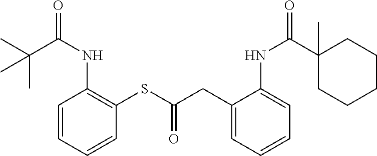 [SMILES: CC(C)(C)C(=O)Nc1ccccc1SC(=O)Cc1ccccc1NC(=O)C1(C)CCCCC1]

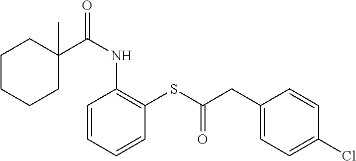 [SMILES: CC1(C(=O)Nc2ccccc2SC(=O)Cc2ccc(Cl)cc2)CCCCC1]

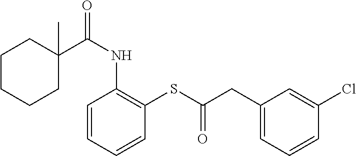 [SMILES: CC1(C(=O)Nc2ccccc2SC(=O)Cc2cccc(Cl)c2)CCCCC1]

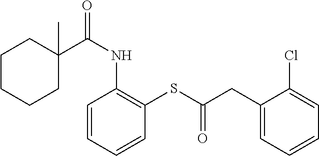 [SMILES: CC1(C(=O)Nc2ccccc2SC(=O)Cc2ccccc2Cl)CCCCC1]

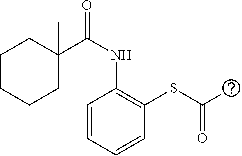 [SMILES: CC(=O)Sc1ccccc1NC(=O)C1(C)CCCCC1]

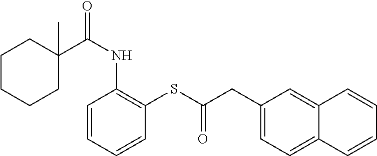 [SMILES: CC1(C(=O)Nc2ccccc2SC(=O)Cc2ccc3ccccc3c2)CCCCC1]

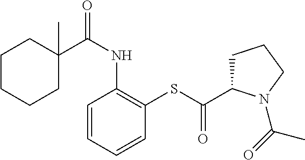 [SMILES: CC(=O)N1CCC[C@H]1C(=O)Sc1ccccc1NC(=O)C1(C)CCCCC1]

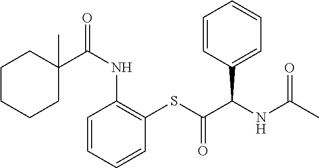 [SMILES: CC(=O)N[C@@H](C(=O)Sc1ccccc1NC(=O)C1(C)CCCCC1)c1ccccc1]

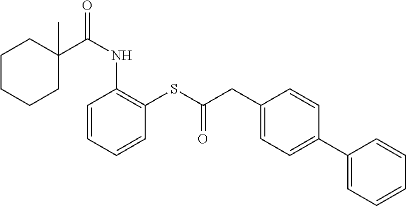 [SMILES: CC1(C(=O)Nc2ccccc2SC(=O)Cc2ccc(-c3ccccc3)cc2)CCCCC1]

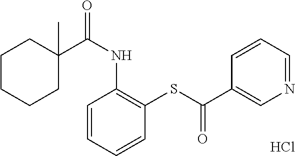 [SMILES: CC1(C(=O)Nc2ccccc2SC(=O)c2cccnc2)CCCCC1 ; Cl]

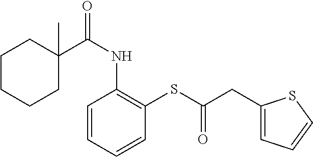 [SMILES: CC1(C(=O)Nc2ccccc2SC(=O)Cc2cccs2)CCCCC1]

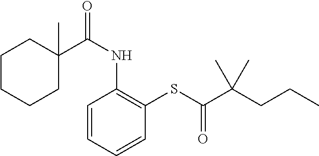 [SMILES: CCCC(C)(C)C(=O)Sc1ccccc1NC(=O)C1(C)CCCCC1]

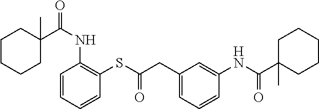 [SMILES: CC1(C(=O)Nc2cccc(CC(=O)Sc3ccccc3NC(=O)C3(C)CCCCC3)c2)CCCCC1]

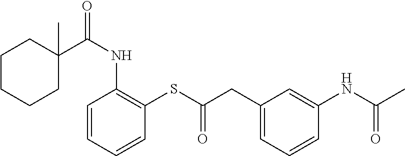 [SMILES: CC(=O)Nc1cccc(CC(=O)Sc2ccccc2NC(=O)C2(C)CCCCC2)c1]

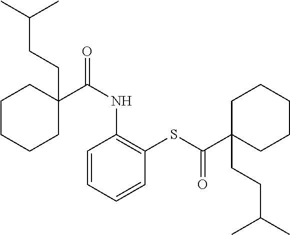 [SMILES: CC(C)CCC1(C(=O)Nc2ccccc2SC(=O)C2(CCC(C)C)CCCCC2)CCCCC1]

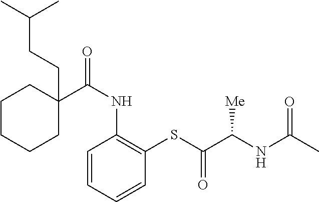 [SMILES: CC(=O)N[C@@H](C)C(=O)Sc1ccccc1NC(=O)C1(CCC(C)C)CCCCC1]

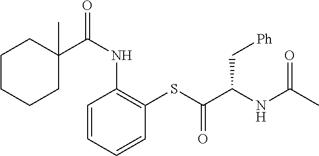 [SMILES: CC(=O)N[C@@H](Cc1ccccc1)C(=O)Sc1ccccc1NC(=O)C1(C)CCCCC1]

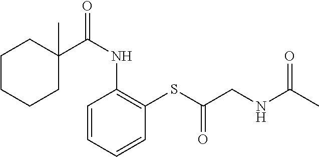 [SMILES: CC(=O)NCC(=O)Sc1ccccc1NC(=O)C1(C)CCCCC1]

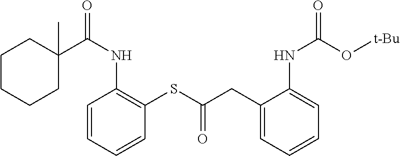 [SMILES: CC(C)(C)OC(=O)Nc1ccccc1CC(=O)Sc1ccccc1NC(=O)C1(C)CCCCC1]

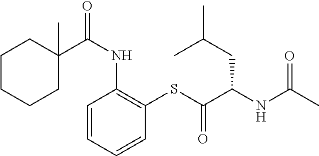 [SMILES: CC(=O)N[C@@H](CC(C)C)C(=O)Sc1ccccc1NC(=O)C1(C)CCCCC1]

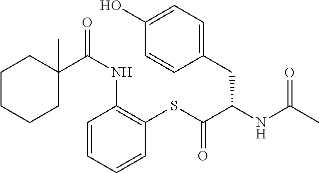 [SMILES: CC(=O)N[C@@H](Cc1ccc(O)cc1)C(=O)Sc1ccccc1NC(=O)C1(C)CCCCC1]

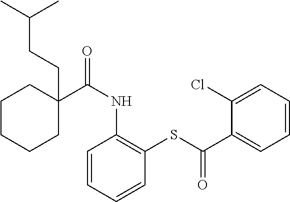 [SMILES: CC(C)CCC1(C(=O)Nc2ccccc2SC(=O)c2ccccc2Cl)CCCCC1]

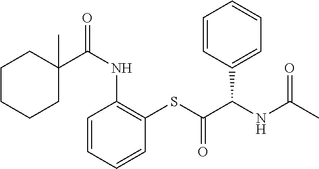 [SMILES: CC(=O)N[C@H](C(=O)Sc1ccccc1NC(=O)C1(C)CCCCC1)c1ccccc1]

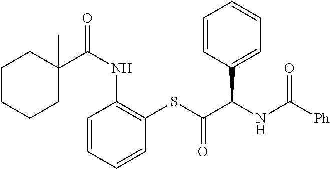 [SMILES: CC1(C(=O)Nc2ccccc2SC(=O)[C@H](NC(=O)c2ccccc2)c2ccccc2)CCCCC1]

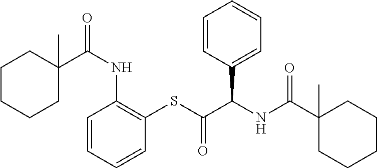 [SMILES: CC1(C(=O)Nc2ccccc2SC(=O)[C@H](NC(=O)C2(C)CCCCC2)c2ccccc2)CCCCC1]

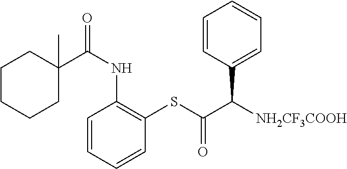

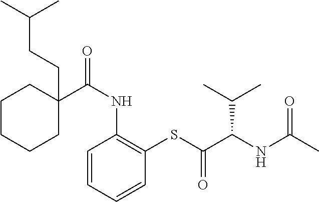 [SMILES: CC(=O)N[C@H](C(=O)Sc1ccccc1NC(=O)C1(CCC(C)C)CCCCC1)C(C)C]

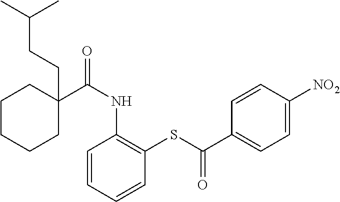 [SMILES: CC(C)CCC1(C(=O)Nc2ccccc2SC(=O)c2ccc([N+](=O)[O-])cc2)CCCCC1]

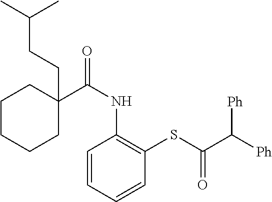 [SMILES: CC(C)CCC1(C(=O)Nc2ccccc2SC(=O)C(c2ccccc2)c2ccccc2)CCCCC1]

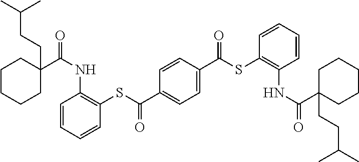 [SMILES: CC(C)CCC1(C(=O)Nc2ccccc2SC(=O)c2ccc(C(=O)Sc3ccccc3NC(=O)C3(CCC(C)C)CCCCC3)cc2)CCCCC1]

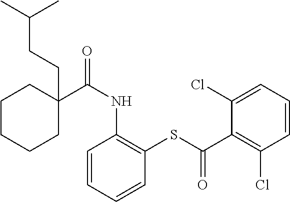 [SMILES: CC(C)CCC1(C(=O)Nc2ccccc2SC(=O)c2c(Cl)cccc2Cl)CCCCC1]

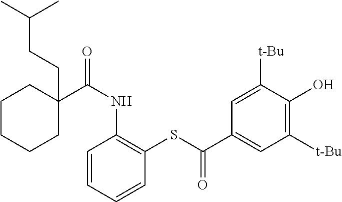 [SMILES: CC(C)CCC1(C(=O)Nc2ccccc2SC(=O)c2cc(C(C)(C)C)c(O)c(C(C)(C)C)c2)CCCCC1]

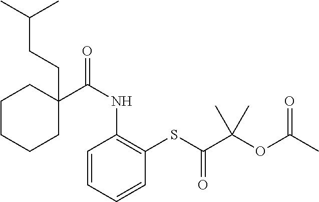 [SMILES: CC(=O)OC(C)(C)C(=O)Sc1ccccc1NC(=O)C1(CCC(C)C)CCCCC1]

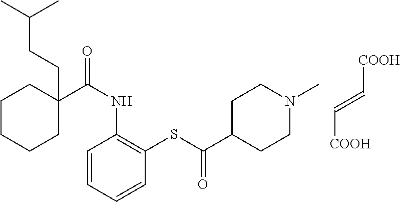

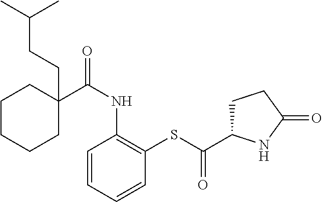 [SMILES: CC(C)CCC1(C(=O)Nc2ccccc2SC(=O)[C@@H]2CCC(=O)N2)CCCCC1]

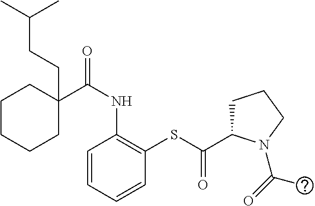 [SMILES: CC(=O)N1CCC[C@H]1C(=O)Sc1ccccc1NC(=O)C1(CCC(C)C)CCCCC1]

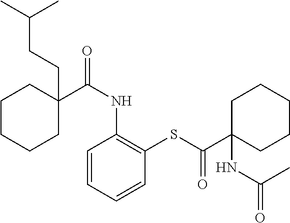 [SMILES: CC(=O)NC1(C(=O)Sc2ccccc2NC(=O)C2(CCC(C)C)CCCCC2)CCCCC1]

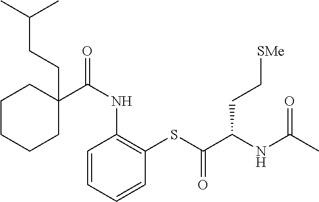 [SMILES: CSCC[C@H](NC(C)=O)C(=O)Sc1ccccc1NC(=O)C1(CCC(C)C)CCCCC1]

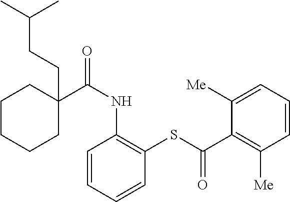 [SMILES: Cc1cccc(C)c1C(=O)Sc1ccccc1NC(=O)C1(CCC(C)C)CCCCC1]

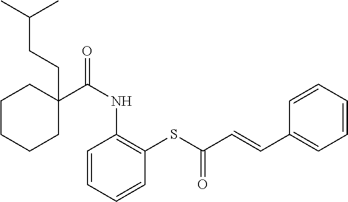 [SMILES: CC(C)CCC1(C(=O)Nc2ccccc2SC(=O)/C=C/c2ccccc2)CCCCC1]

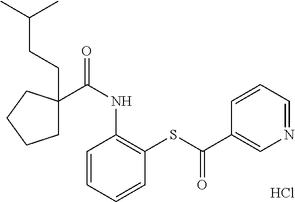 [SMILES: CC(C)CCC1(C(=O)Nc2ccccc2SC(=O)c2cccnc2)CCCC1 ; Cl]

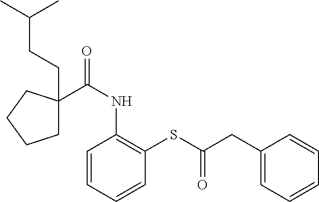 [SMILES: CC(C)CCC1(C(=O)Nc2ccccc2SC(=O)Cc2ccccc2)CCCC1]

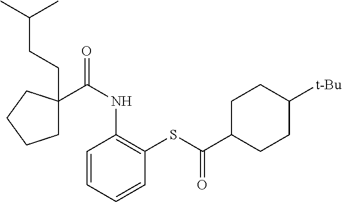 [SMILES: CC(C)CCC1(C(=O)Nc2ccccc2SC(=O)C2CCC(C(C)(C)C)CC2)CCCC1]

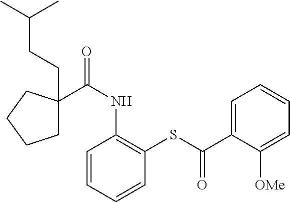 [SMILES: COc1ccccc1C(=O)Sc1ccccc1NC(=O)C1(CCC(C)C)CCCC1]

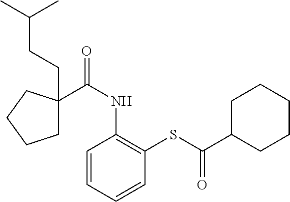 [SMILES: CC(C)CCC1(C(=O)Nc2ccccc2SC(=O)C2CCCCC2)CCCC1]

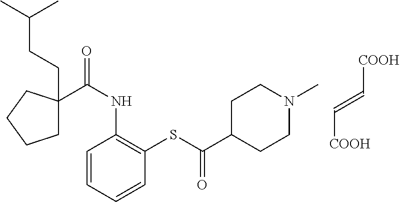

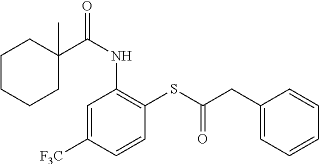 [SMILES: CC1(C(=O)Nc2cc(C(F)(F)F)ccc2SC(=O)Cc2ccccc2)CCCCC1]

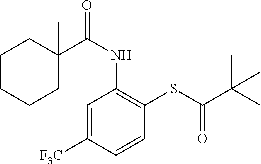 [SMILES: CC(C)(C)C(=O)Sc1ccc(C(F)(F)F)cc1NC(=O)C1(C)CCCCC1]

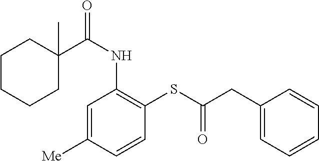 [SMILES: Cc1ccc(SC(=O)Cc2ccccc2)c(NC(=O)C2(C)CCCCC2)c1]

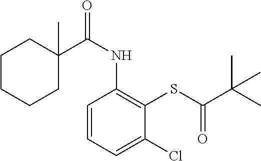 [SMILES: CC(C)(C)C(=O)Sc1c(Cl)cccc1NC(=O)C1(C)CCCCC1]

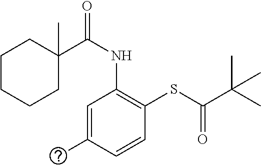 [SMILES: Cc1ccc(SC(=O)C(C)(C)C)c(NC(=O)C2(C)CCCCC2)c1]

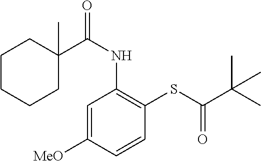 [SMILES: COc1ccc(SC(=O)C(C)(C)C)c(NC(=O)C2(C)CCCCC2)c1]

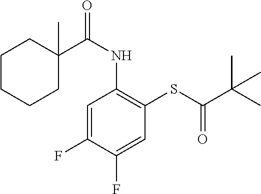 [SMILES: CC(C)(C)C(=O)Sc1cc(F)c(F)cc1NC(=O)C1(C)CCCCC1]

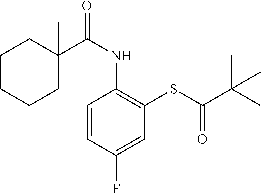 [SMILES: CC(C)(C)C(=O)Sc1cc(F)ccc1NC(=O)C1(C)CCCCC1]

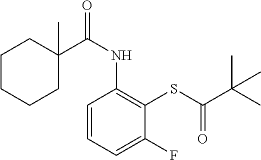 [SMILES: CC(C)(C)C(=O)Sc1c(F)cccc1NC(=O)C1(C)CCCCC1]

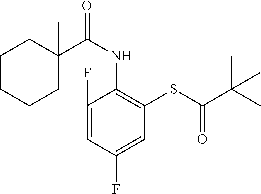 [SMILES: CC(C)(C)C(=O)Sc1cc(F)cc(F)c1NC(=O)C1(C)CCCCC1]

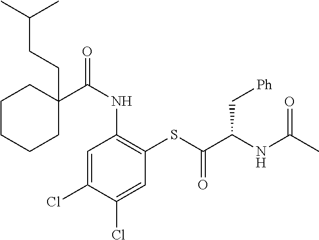 [SMILES: CC(=O)N[C@@H](Cc1ccccc1)C(=O)Sc1cc(Cl)c(Cl)cc1NC(=O)C1(CCC(C)C)CCCCC1]

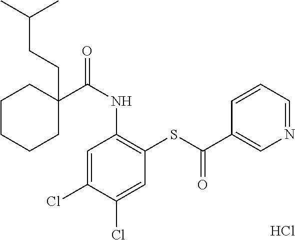 [SMILES: CC(C)CCC1(C(=O)Nc2cc(Cl)c(Cl)cc2SC(=O)c2cccnc2)CCCCC1 ; Cl]

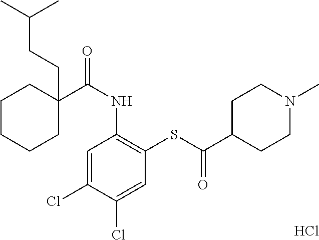 [SMILES: CC(C)CCC1(C(=O)Nc2cc(Cl)c(Cl)cc2SC(=O)C2CCN(C)CC2)CCCCC1 ; Cl]

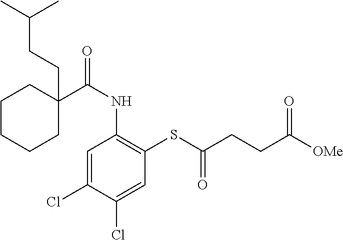 [SMILES: COC(=O)CCC(=O)Sc1cc(Cl)c(Cl)cc1NC(=O)C1(CCC(C)C)CCCCC1]

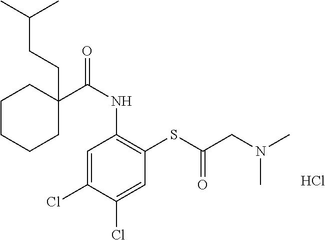 [SMILES: CC(C)CCC1(C(=O)Nc2cc(Cl)c(Cl)cc2SC(=O)CN(C)C)CCCCC1 ; Cl]

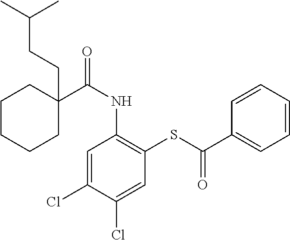 [SMILES: CC(C)CCC1(C(=O)Nc2cc(Cl)c(Cl)cc2SC(=O)c2ccccc2)CCCCC1]

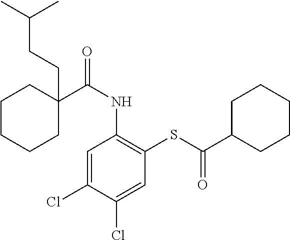 [SMILES: CC(C)CCC1(C(=O)Nc2cc(Cl)c(Cl)cc2SC(=O)C2CCCCC2)CCCCC1]

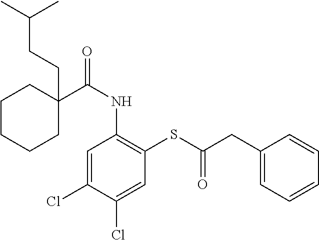 [SMILES: CC(C)CCC1(C(=O)Nc2cc(Cl)c(Cl)cc2SC(=O)Cc2ccccc2)CCCCC1]

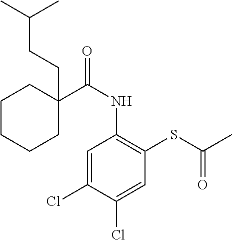 [SMILES: CC(=O)Sc1cc(Cl)c(Cl)cc1NC(=O)C1(CCC(C)C)CCCCC1]

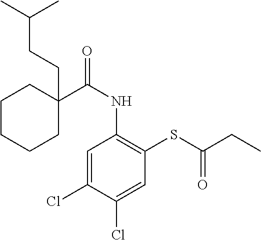 [SMILES: CCC(=O)Sc1cc(Cl)c(Cl)cc1NC(=O)C1(CCC(C)C)CCCCC1]

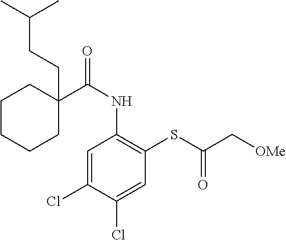 [SMILES: COCC(=O)Sc1cc(Cl)c(Cl)cc1NC(=O)C1(CCC(C)C)CCCCC1]

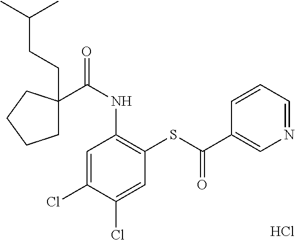 [SMILES: CC(C)CCC1(C(=O)Nc2cc(Cl)c(Cl)cc2SC(=O)c2cccnc2)CCCC1 ; Cl]

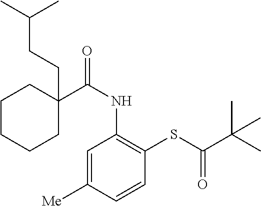 [SMILES: Cc1ccc(SC(=O)C(C)(C)C)c(NC(=O)C2(CCC(C)C)CCCCC2)c1]

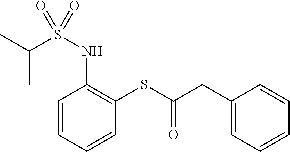 [SMILES: CC(C)S(=O)(=O)Nc1ccccc1SC(=O)Cc1ccccc1]

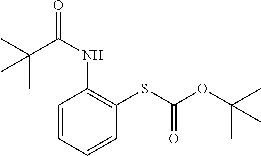 [SMILES: CC(C)(C)OC(=O)Sc1ccccc1NC(=O)C(C)(C)C]

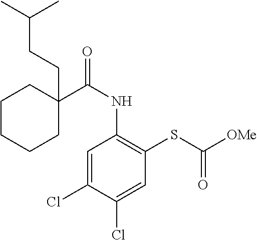 [SMILES: COC(=O)Sc1cc(Cl)c(Cl)cc1NC(=O)C1(CCC(C)C)CCCCC1]

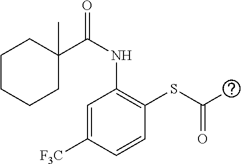 [SMILES: CC(=O)Sc1ccc(C(F)(F)F)cc1NC(=O)C1(C)CCCCC1]

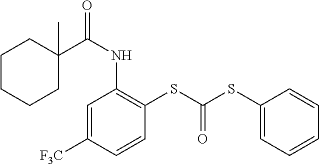 [SMILES: CC1(C(=O)Nc2cc(C(F)(F)F)ccc2SC(=O)Sc2ccccc2)CCCCC1]

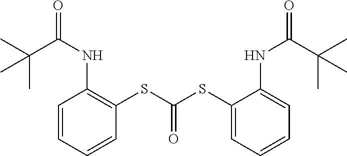 [SMILES: CC(C)(C)C(=O)Nc1ccccc1SC(=O)Sc1ccccc1NC(=O)C(C)(C)C]

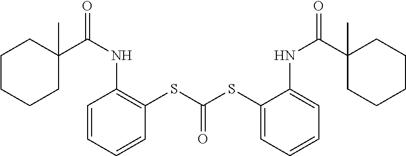 [SMILES: CC1(C(=O)Nc2ccccc2SC(=O)Sc2ccccc2NC(=O)C2(C)CCCCC2)CCCCC1]

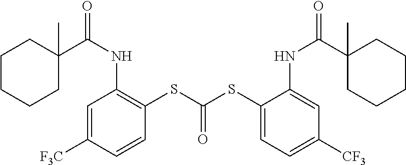 [SMILES: CC1(C(=O)Nc2cc(C(F)(F)F)ccc2SC(=O)Sc2ccc(C(F)(F)F)cc2NC(=O)C2(C)CCCCC2)CCCCC1]

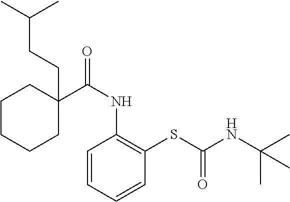 [SMILES: CC(C)CCC1(C(=O)Nc2ccccc2SC(=O)NC(C)(C)C)CCCCC1]

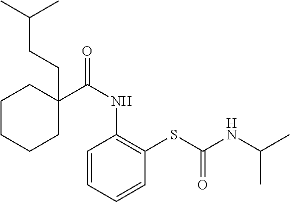 [SMILES: CC(C)CCC1(C(=O)Nc2ccccc2SC(=O)NC(C)C)CCCCC1]

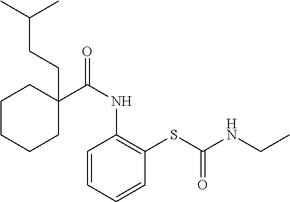 [SMILES: CCNC(=O)Sc1ccccc1NC(=O)C1(CCC(C)C)CCCCC1]

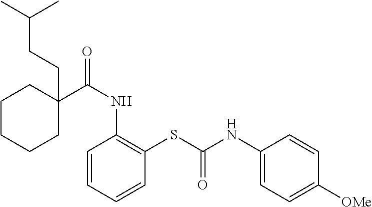 [SMILES: COc1ccc(NC(=O)Sc2ccccc2NC(=O)C2(CCC(C)C)CCCCC2)cc1]

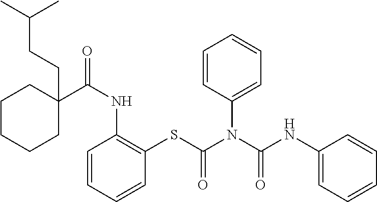 [SMILES: CC(C)CCC1(C(=O)Nc2ccccc2SC(=O)N(C(=O)Nc2ccccc2)c2ccccc2)CCCCC1]

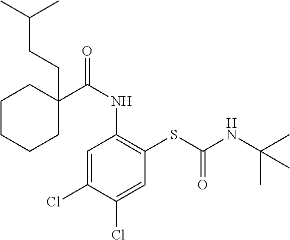 [SMILES: CC(C)CCC1(C(=O)Nc2cc(Cl)c(Cl)cc2SC(=O)NC(C)(C)C)CCCCC1]

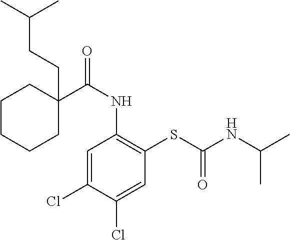 [SMILES: CC(C)CCC1(C(=O)Nc2cc(Cl)c(Cl)cc2SC(=O)NC(C)C)CCCCC1]

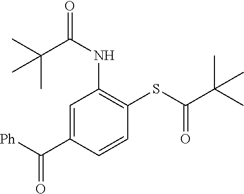 [SMILES: CC(C)(C)C(=O)Nc1cc(C(=O)c2ccccc2)ccc1SC(=O)C(C)(C)C]

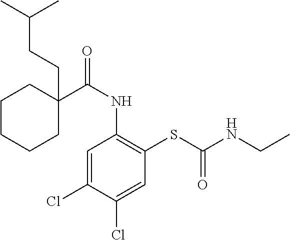 [SMILES: CCNC(=O)Sc1cc(Cl)c(Cl)cc1NC(=O)C1(CCC(C)C)CCCCC1]

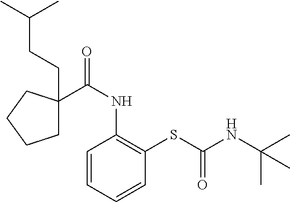 [SMILES: CC(C)CCC1(C(=O)Nc2ccccc2SC(=O)NC(C)(C)C)CCCC1]

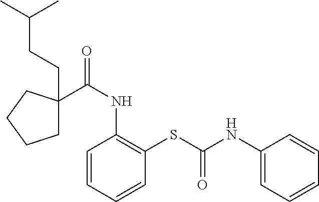 [SMILES: CC(C)CCC1(C(=O)Nc2ccccc2SC(=O)Nc2ccccc2)CCCC1]

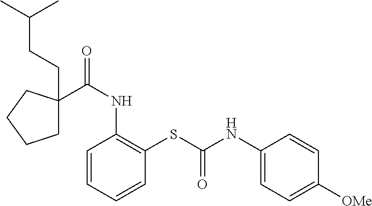 [SMILES: COc1ccc(NC(=O)Sc2ccccc2NC(=O)C2(CCC(C)C)CCCC2)cc1]

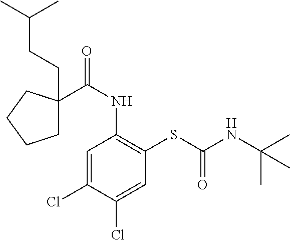 [SMILES: CC(C)CCC1(C(=O)Nc2cc(Cl)c(Cl)cc2SC(=O)NC(C)(C)C)CCCC1]

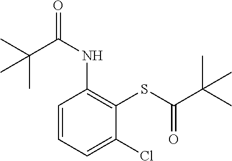 [SMILES: CC(C)(C)C(=O)Nc1cccc(Cl)c1SC(=O)C(C)(C)C]

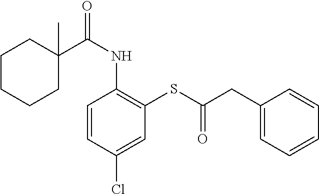 [SMILES: CC1(C(=O)Nc2ccc(Cl)cc2SC(=O)Cc2ccccc2)CCCCC1]

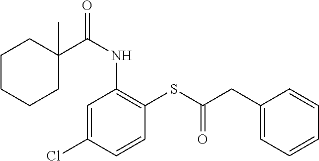 [SMILES: CC1(C(=O)Nc2cc(Cl)ccc2SC(=O)Cc2ccccc2)CCCCC1]

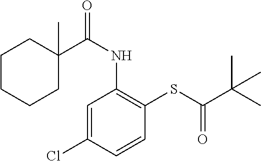 [SMILES: CC(C)(C)C(=O)Sc1ccc(Cl)cc1NC(=O)C1(C)CCCCC1]

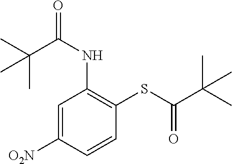 [SMILES: CC(C)(C)C(=O)Nc1cc([N+](=O)[O-])ccc1SC(=O)C(C)(C)C]

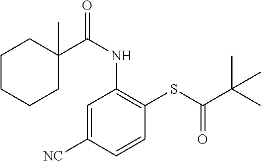 [SMILES: CC(C)(C)C(=O)Sc1ccc(C#N)cc1NC(=O)C1(C)CCCCC1]

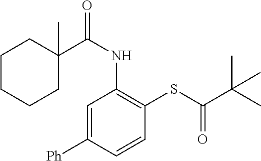 [SMILES: CC(C)(C)C(=O)Sc1ccc(-c2ccccc2)cc1NC(=O)C1(C)CCCCC1]

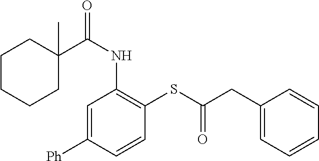 [SMILES: CC1(C(=O)Nc2cc(-c3ccccc3)ccc2SC(=O)Cc2ccccc2)CCCCC1]

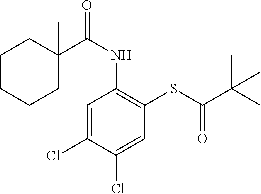 [SMILES: CC(C)(C)C(=O)Sc1cc(Cl)c(Cl)cc1NC(=O)C1(C)CCCCC1]

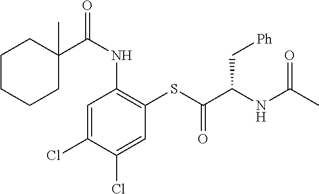 [SMILES: CC(=O)N[C@@H](Cc1ccccc1)C(=O)Sc1cc(Cl)c(Cl)cc1NC(=O)C1(C)CCCCC1]

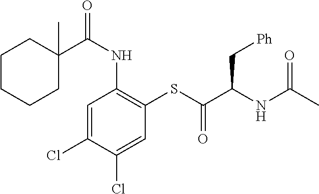 [SMILES: CC(=O)N[C@H](Cc1ccccc1)C(=O)Sc1cc(Cl)c(Cl)cc1NC(=O)C1(C)CCCCC1]

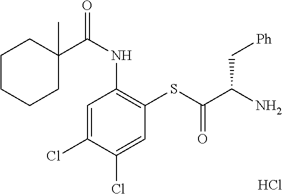 [SMILES: CC1(C(=O)Nc2cc(Cl)c(Cl)cc2SC(=O)[C@@H](N)Cc2ccccc2)CCCCC1 ; Cl]

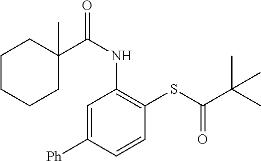 [SMILES: CC(C)(C)C(=O)Sc1ccc(-c2ccccc2)cc1NC(=O)C1(C)CCCCC1]

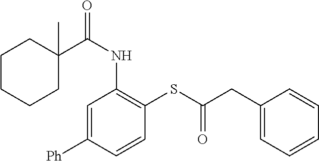 [SMILES: CC1(C(=O)Nc2cc(-c3ccccc3)ccc2SC(=O)Cc2ccccc2)CCCCC1]

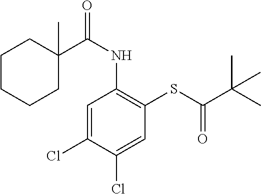 [SMILES: CC(C)(C)C(=O)Sc1cc(Cl)c(Cl)cc1NC(=O)C1(C)CCCCC1]

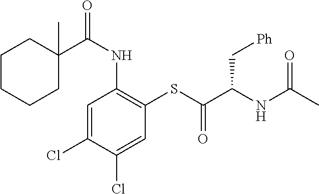 [SMILES: CC(=O)N[C@@H](Cc1ccccc1)C(=O)Sc1cc(Cl)c(Cl)cc1NC(=O)C1(C)CCCCC1]

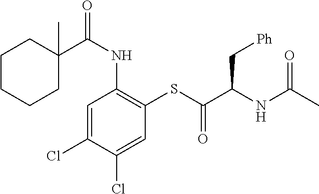 [SMILES: CC(=O)N[C@H](Cc1ccccc1)C(=O)Sc1cc(Cl)c(Cl)cc1NC(=O)C1(C)CCCCC1]

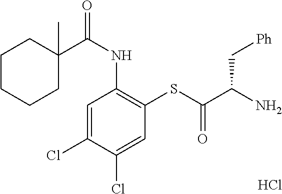 [SMILES: CC1(C(=O)Nc2cc(Cl)c(Cl)cc2SC(=O)[C@@H](N)Cc2ccccc2)CCCCC1 ; Cl]

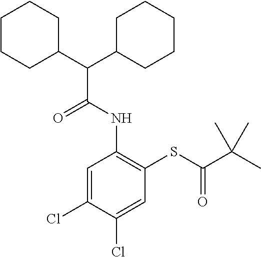 [SMILES: CC(C)(C)C(=O)Sc1cc(Cl)c(Cl)cc1NC(=O)C(C1CCCCC1)C1CCCCC1]

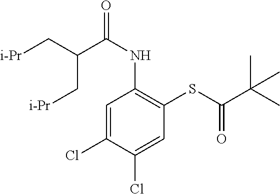 [SMILES: CC(C)CC(CC(C)C)C(=O)Nc1cc(Cl)c(Cl)cc1SC(=O)C(C)(C)C]

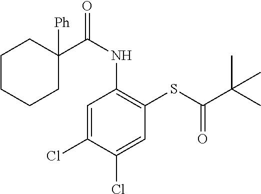 [SMILES: CC(C)(C)C(=O)Sc1cc(Cl)c(Cl)cc1NC(=O)C1(c2ccccc2)CCCCC1]

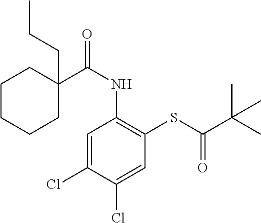 [SMILES: CCCC1(C(=O)Nc2cc(Cl)c(Cl)cc2SC(=O)C(C)(C)C)CCCCC1]

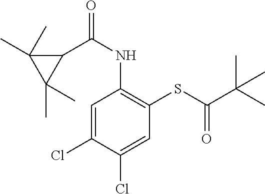 [SMILES: CC(C)(C)C(=O)Sc1cc(Cl)c(Cl)cc1NC(=O)C1C(C)(C)C1(C)C]

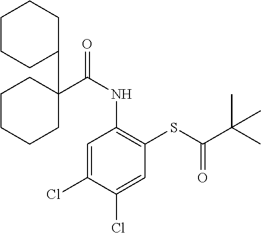 [SMILES: CC(C)(C)C(=O)Sc1cc(Cl)c(Cl)cc1NC(=O)C1(C2CCCCC2)CCCCC1]

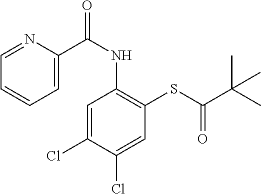 [SMILES: CC(C)(C)C(=O)Sc1cc(Cl)c(Cl)cc1NC(=O)c1ccccn1]

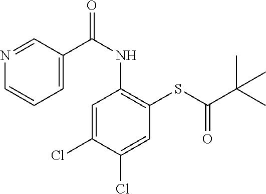 [SMILES: CC(C)(C)C(=O)Sc1cc(Cl)c(Cl)cc1NC(=O)c1cccnc1]

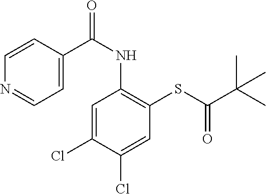 [SMILES: CC(C)(C)C(=O)Sc1cc(Cl)c(Cl)cc1NC(=O)c1ccncc1]

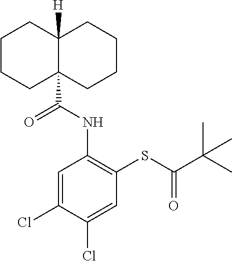 [SMILES: CC(C)(C)C(=O)Sc1cc(Cl)c(Cl)cc1NC(=O)[C@]12CCCC[C@@H]1CCCC2]

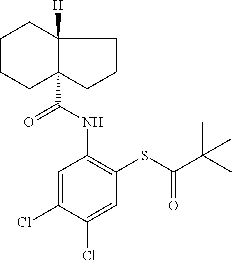 [SMILES: CC(C)(C)C(=O)Sc1cc(Cl)c(Cl)cc1NC(=O)[C@]12CCCC[C@@H]1CCC2]

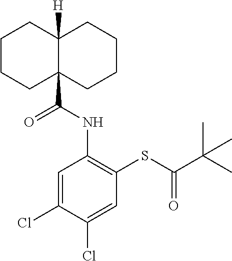 [SMILES: CC(C)(C)C(=O)Sc1cc(Cl)c(Cl)cc1NC(=O)[C@]12CCCC[C@H]1CCCC2]

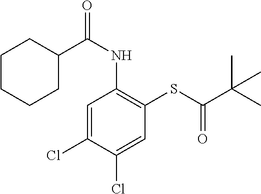 [SMILES: CC(C)(C)C(=O)Sc1cc(Cl)c(Cl)cc1NC(=O)C1CCCCC1]

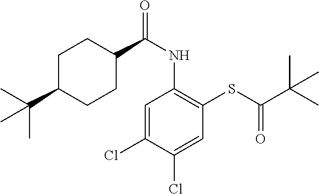

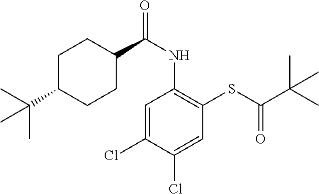

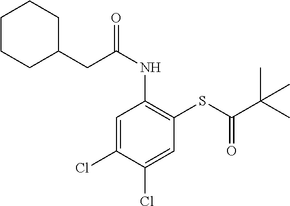 [SMILES: CC(C)(C)C(=O)Sc1cc(Cl)c(Cl)cc1NC(=O)CC1CCCCC1]

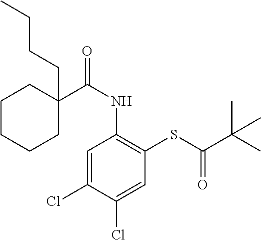 [SMILES: CCCCC1(C(=O)Nc2cc(Cl)c(Cl)cc2SC(=O)C(C)(C)C)CCCCC1]

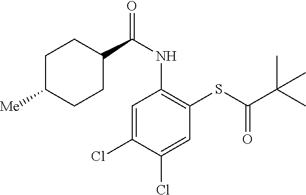

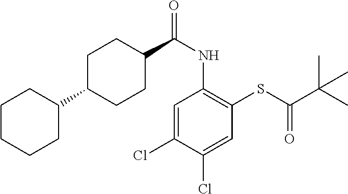

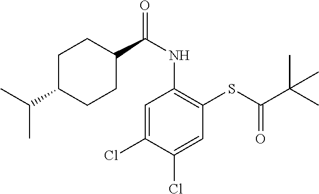

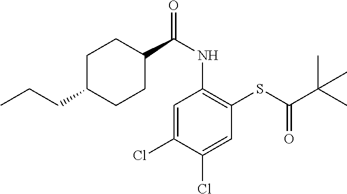

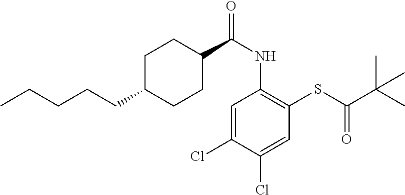

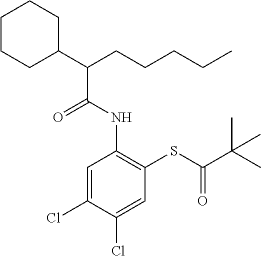 [SMILES: CCCCCC(C(=O)Nc1cc(Cl)c(Cl)cc1SC(=O)C(C)(C)C)C1CCCCC1]

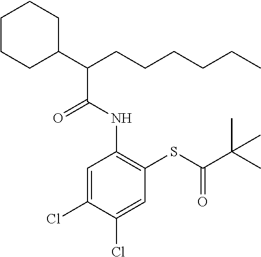 [SMILES: CCCCCCC(C(=O)Nc1cc(Cl)c(Cl)cc1SC(=O)C(C)(C)C)C1CCCCC1]

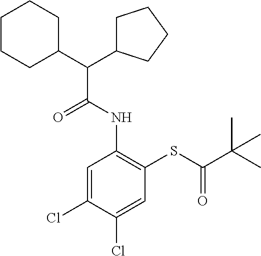 [SMILES: CC(C)(C)C(=O)Sc1cc(Cl)c(Cl)cc1NC(=O)C(C1CCCCC1)C1CCCC1]

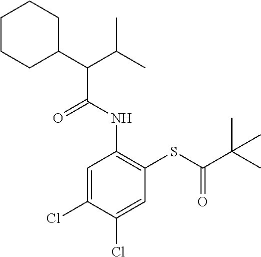 [SMILES: CC(C)C(C(=O)Nc1cc(Cl)c(Cl)cc1SC(=O)C(C)(C)C)C1CCCCC1]

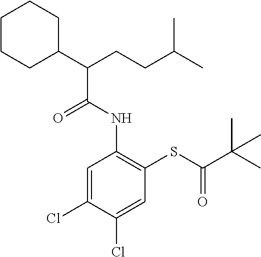 [SMILES: CC(C)CCC(C(=O)Nc1cc(Cl)c(Cl)cc1SC(=O)C(C)(C)C)C1CCCCC1]

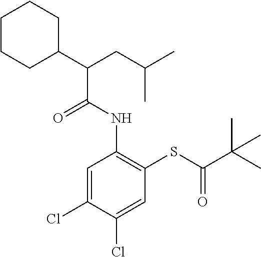 [SMILES: CC(C)CC(C(=O)Nc1cc(Cl)c(Cl)cc1SC(=O)C(C)(C)C)C1CCCCC1]

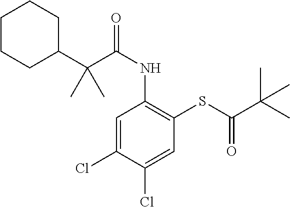 [SMILES: CC(C)(C)C(=O)Sc1cc(Cl)c(Cl)cc1NC(=O)C(C)(C)C1CCCCC1]

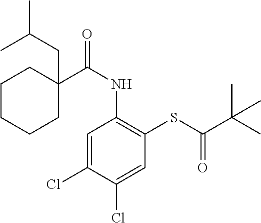 [SMILES: CC(C)CC1(C(=O)Nc2cc(Cl)c(Cl)cc2SC(=O)C(C)(C)C)CCCCC1]

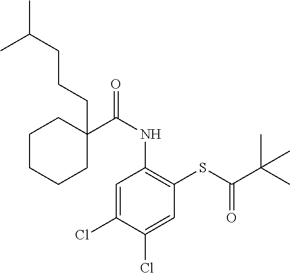 [SMILES: CC(C)CCCC1(C(=O)Nc2cc(Cl)c(Cl)cc2SC(=O)C(C)(C)C)CCCCC1]

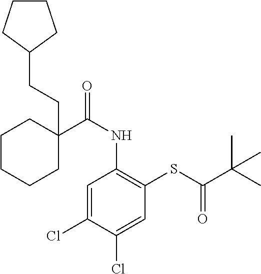 [SMILES: CC(C)(C)C(=O)Sc1cc(Cl)c(Cl)cc1NC(=O)C1(CCC2CCCC2)CCCCC1]

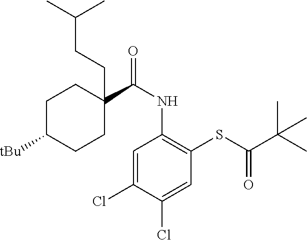

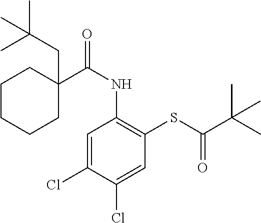 [SMILES: CC(C)(C)CC1(C(=O)Nc2cc(Cl)c(Cl)cc2SC(=O)C(C)(C)C)CCCCC1]

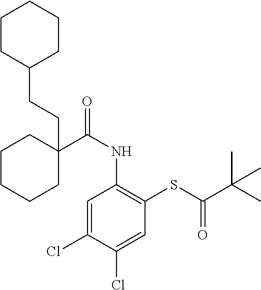 [SMILES: CC(C)(C)C(=O)Sc1cc(Cl)c(Cl)cc1NC(=O)C1(CCC2CCCCC2)CCCCC1]

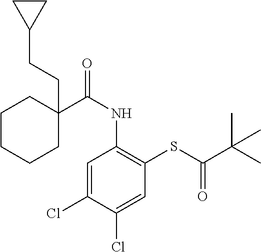 [SMILES: CC(C)(C)C(=O)Sc1cc(Cl)c(Cl)cc1NC(=O)C1(CCC2CC2)CCCCC1]

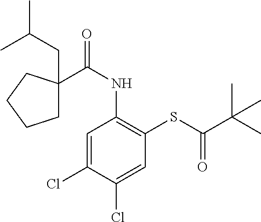 [SMILES: CC(C)CC1(C(=O)Nc2cc(Cl)c(Cl)cc2SC(=O)C(C)(C)C)CCCC1]

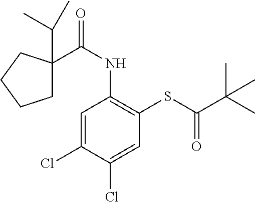 [SMILES: CC(C)C1(C(=O)Nc2cc(Cl)c(Cl)cc2SC(=O)C(C)(C)C)CCCC1]

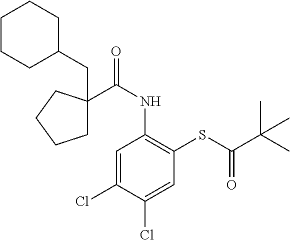 [SMILES: CC(C)(C)C(=O)Sc1cc(Cl)c(Cl)cc1NC(=O)C1(CC2CCCCC2)CCCC1]

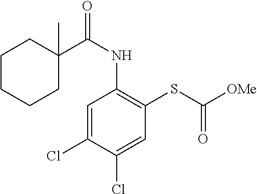 [SMILES: COC(=O)Sc1cc(Cl)c(Cl)cc1NC(=O)C1(C)CCCCC1]

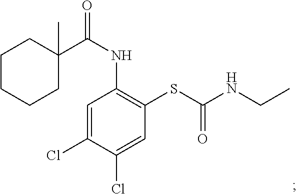 [SMILES: CCNC(=O)Sc1cc(Cl)c(Cl)cc1NC(=O)C1(C)CCCCC1]

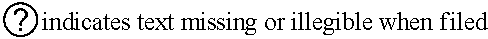

or a pharmaceutically acceptable salt, hydrate, or solvate thereof.

In some embodiments, the antiviral compound has any one of the structures shown in the following table:

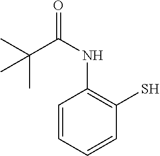

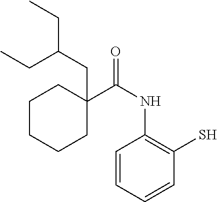 [SMILES: CCC(CC)CC1(C(=O)Nc2ccccc2S)CCCCC1]

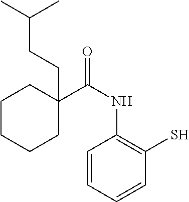 [SMILES: CC(C)CCC1(C(=O)Nc2ccccc2S)CCCCC1]

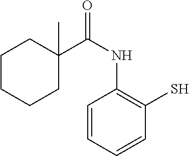 [SMILES: CC1(C(=O)Nc2ccccc2S)CCCCC1]

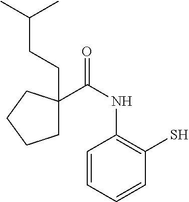 [SMILES: CC(C)CCC1(C(=O)Nc2ccccc2S)CCCC1]

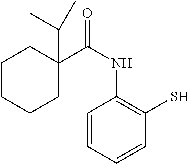 [SMILES: CC(C)C1(C(=O)Nc2ccccc2S)CCCCC1]

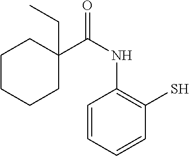 [SMILES: CCC1(C(=O)Nc2ccccc2S)CCCCC1]

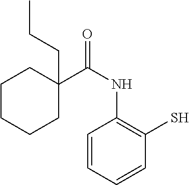 [SMILES: CCCC1(C(=O)Nc2ccccc2S)CCCCC1]

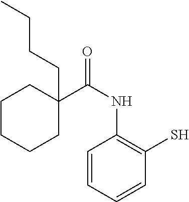 [SMILES: CCCCC1(C(=O)Nc2ccccc2S)CCCCC1]

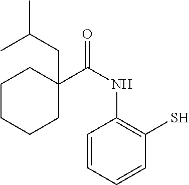 [SMILES: CC(C)CC1(C(=O)Nc2ccccc2S)CCCCC1]

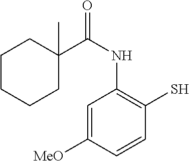 [SMILES: COc1ccc(S)c(NC(=O)C2(C)CCCCC2)c1]

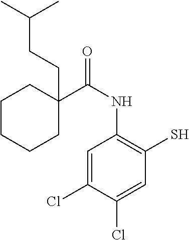 [SMILES: CC(C)CCC1(C(=O)Nc2cc(Cl)c(Cl)cc2S)CCCCC1]

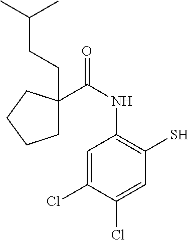 [SMILES: CC(C)CCC1(C(=O)Nc2cc(Cl)c(Cl)cc2S)CCCC1]

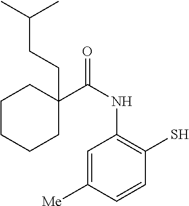 [SMILES: Cc1ccc(S)c(NC(=O)C2(CCC(C)C)CCCCC2)c1]

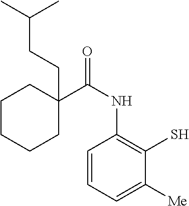 [SMILES: Cc1cccc(NC(=O)C2(CCC(C)C)CCCCC2)c1S]

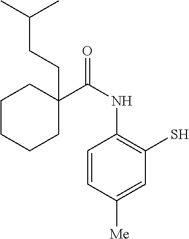 [SMILES: Cc1ccc(NC(=O)C2(CCC(C)C)CCCCC2)c(S)c1]

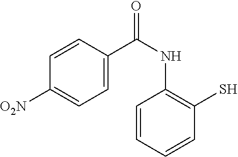 [SMILES: O=C(Nc1ccccc1S)c1ccc([N+](=O)[O-])cc1]

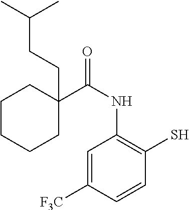 [SMILES: CC(C)CCC1(C(=O)Nc2cc(C(F)(F)F)ccc2S)CCCCC1]

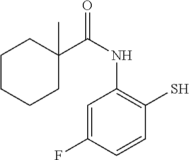 [SMILES: CC1(C(=O)Nc2cc(F)ccc2S)CCCCC1]

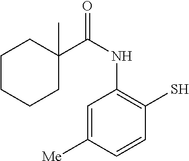 [SMILES: Cc1ccc(S)c(NC(=O)C2(C)CCCCC2)c1]

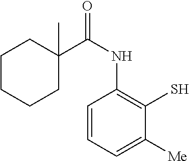 [SMILES: Cc1cccc(NC(=O)C2(C)CCCCC2)c1S]

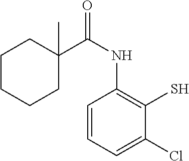 [SMILES: CC1(C(=O)Nc2cccc(Cl)c2S)CCCCC1]

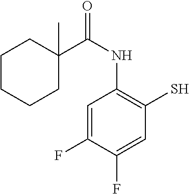 [SMILES: CC1(C(=O)Nc2cc(F)c(F)cc2S)CCCCC1]

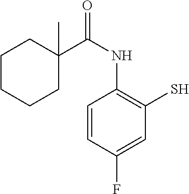 [SMILES: CC1(C(=O)Nc2ccc(F)cc2S)CCCCC1]

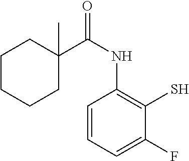 [SMILES: CC1(C(=O)Nc2cccc(F)c2S)CCCCC1]

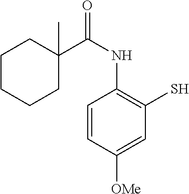 [SMILES: COc1ccc(NC(=O)C2(C)CCCCC2)c(S)c1]

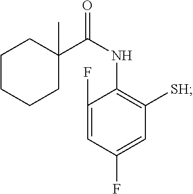 [SMILES: CC1(C(=O)Nc2c(F)cc(F)cc2S)CCCCC1]

or a pharmaceutically acceptable salt, hydrate or solvate thereof.

In some embodiments, the antiviral compound has any one of the following structures:

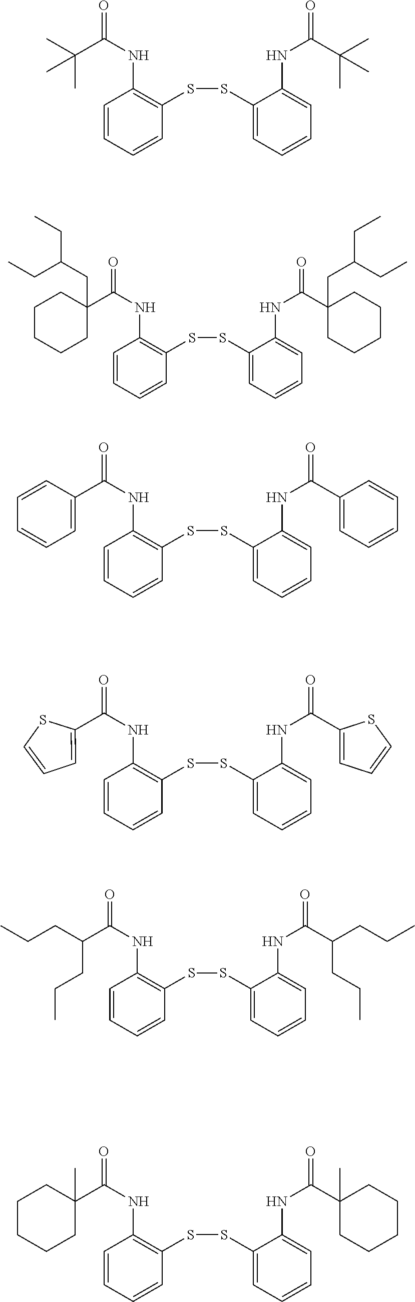

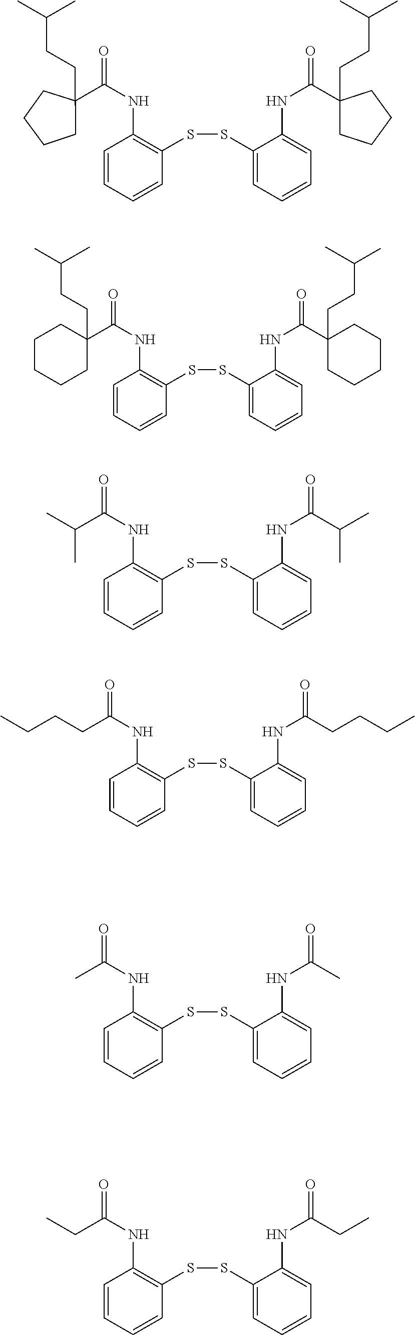

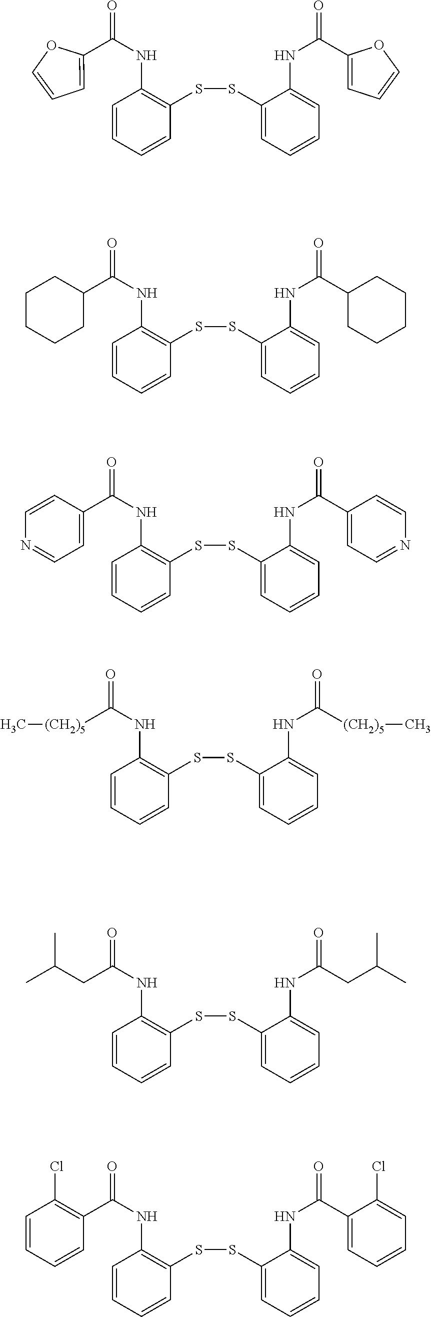

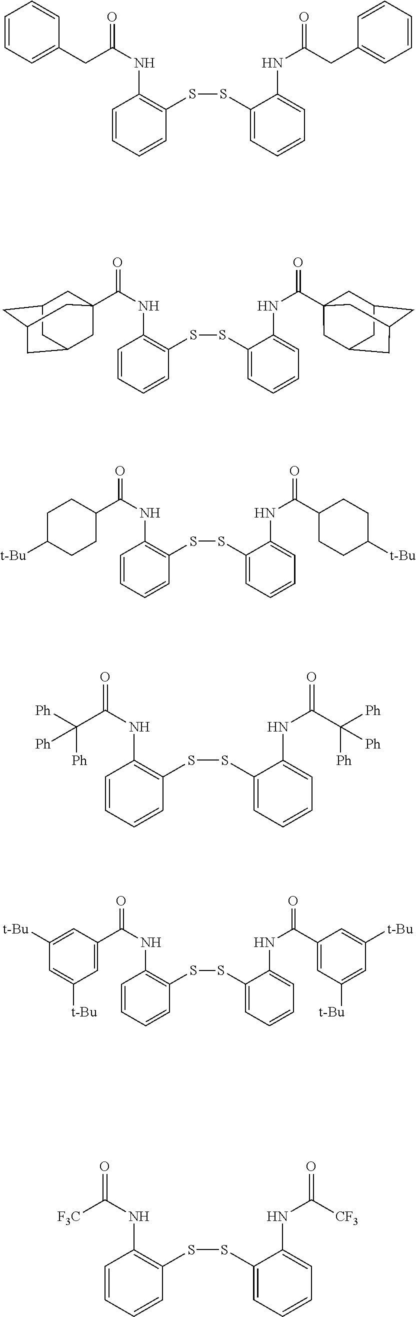 [SMILES: O=C(Cc1ccccc1)Nc1ccccc1SSc1ccccc1NC(=O)Cc1ccccc1 ; O=C(Nc1ccccc1SSc1ccccc1NC(=O)C12CC3CC(CC(C3)C1)C2)C12CC3CC(CC(C3)C1)C2 ; CC(C)(C)C1CCC(C(=O)Nc2ccccc2SSc2ccccc2NC(=O)C2CCC(C(C)(C)C)CC2)CC1 ; O=C(Nc1ccccc1SSc1ccccc1NC(=O)C(c1ccccc1)(c1ccccc1)c1ccccc1)C(c1ccccc1)(c1ccccc1)c1ccccc1 ; CC(C)(C)c1cc(C(=O)Nc2ccccc2SSc2ccccc2NC(=O)c2cc(C(C)(C)C)cc(C(C)(C)C)c2)cc(C(C)(C)C)c1 ; O=C(Nc1ccccc1SSc1ccccc1NC(=O)C(F)(F)F)C(F)(F)F]

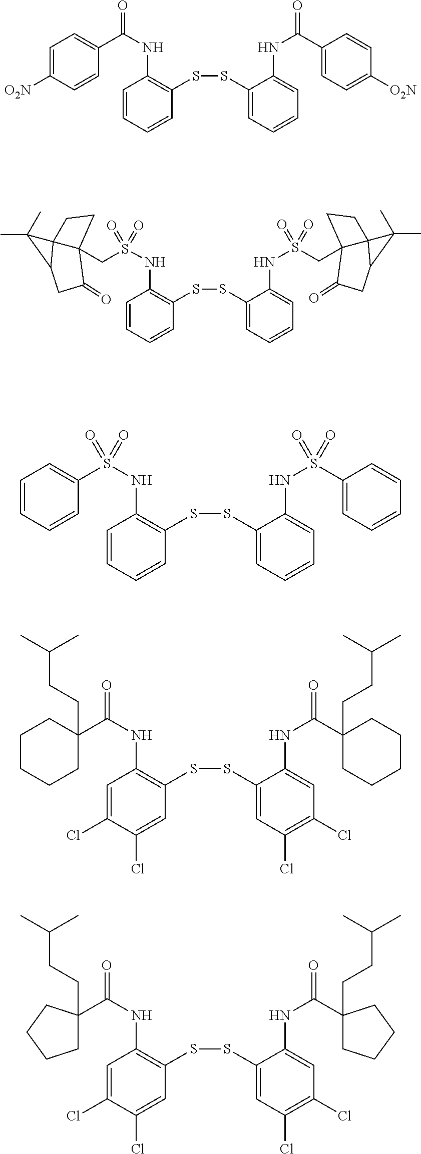

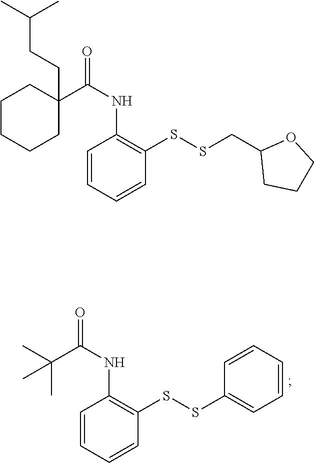 [SMILES: CC(C)CCC1(C(=O)Nc2ccccc2SSCC2CCCO2)CCCCC1 ; CC(C)(C)C(=O)Nc1ccccc1SSc1ccccc1]

or a pharmaceutically acceptable salt, hydrate, or solvate thereof.

### Pharmaceutically Acceptable Salts, Hydrates and Solvates

In some embodiments, the antiviral compound is a pharmaceutically acceptable salt. Pharmaceutically acceptable salts include, for example, acid-addition salts and base-addition salts. The acid that forms an acid-addition salt can be an organic acid or an inorganic acid. A base that forms a base-addition salt can be an organic base or an inorganic base. In some embodiments, a pharmaceutically acceptable salt is a metal salt. In some embodiments, a pharmaceutically acceptable salt is an ammonium salt.

Acid-addition salts can arise from the addition of an acid to the free-base form of an antiviral compound useful in the compositions and methods described herein. In some embodiments, the acid is organic. In some embodiments, the acid is inorganic. Non-limiting examples of suitable acids include hydrochloric acid, hydrobromic acid, hydroiodic acid, nitric acid, nitrous acid, sulfuric acid, sulfurous acid, a phosphoric acid, nicotinic acid, isonicotinic acid, lactic acid, salicylic acid, 4-aminosalicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, citric acid, oxalic acid, maleic acid, hydroxymaleic acid, methylmaleic acid, glycolic acid, malic acid, cinnamic acid, mandelic acid, 2-phenoxybenzoic acid, 2-acetoxybenzoic acid, embonic acid, phenylacetic acid, N-cyclohexylsulfamic acid, methanesulfonic acid, ethanesulfonic acid, benzenesulfonic acid, p-toluenesulfonic acid, 2-hydroxyethanesulfonic acid, ethane-1,2-disulfonic acid, 4-methylbenzenesulfonic acid, naphthalene-2-sulfonic acid, naphthalene-1,5-disulfonic acid, 2-phosphoglyceric acid, 3-phosphoglyceric acid, glucose-6-phosphoric acid, and an amino acid.

Non-limiting examples of suitable acid-addition salts include a hydrochloride salt, a hydrobromide salt, a hydroiodide salt, a nitrate salt, a nitrite salt, a sulfate salt, a sulfite salt, a phosphate salt, a hydrogen phosphate salt, a dihydrogen phosphate salt, a carbonate salt, a bicarbonate salt, a nicotinate salt, an isonicotinate salt, a lactate salt, a salicylate salt, a 4-aminosalicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a citrate salt, an oxalate salt, a maleate salt, a hydroxymaleate salt, a methylmaleate salt, a glycolate salt, a malate salt, a cinnamate salt, a mandelate salt, a 2-phenoxybenzoate salt, a 2-acetoxybenzoate salt, an embonate salt, a phenylacetate salt, an N-cyclohexylsulfamate salt, a methanesulfonate salt, an ethanesulfonate salt, a benzenesulfonate salt, a p-toluenesulfonate salt, a 2-hydroxyethanesulfonate salt, an ethane-1,2-disulfonate salt, a 4-methylbenzenesulfonate salt, a naphthalene-2-sulfonate salt, a naphthalene-1,5-disulfonate salt, a 2-phosphoglycerate salt, a 3-phosphoglycerate salt, a glucose-6-phosphate salt, and an amino acid salt.

Metal salts can arise from the addition of an inorganic base to an antiviral compound having a carboxyl group. The inorganic base can include a metal cation paired with a basic counterion, such as, for example, hydroxide, carbonate, bicarbonate, or phosphate. The metal can be an alkali metal, alkaline earth metal, transition metal, or main group metal. Non-limiting examples of suitable metals include lithium, sodium, potassium, cesium, cerium, magnesium, manganese, iron, calcium, strontium, cobalt, titanium, aluminum, copper, cadmium, and zinc.

Non-limiting examples of suitable metal salts include a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, and a zinc salt.

Ammonium salts can arise from the addition of ammonia or an organic amine to an antiviral compound having a carboxyl group. Non-limiting examples of suitable organic amines include triethyl amine, diisopropyl amine, ethanol amine, diethanol amine, triethanol amine, morpholine, N-methylmorpholine, piperidine, N-methylpiperidine, N-ethylpiperidine, dibenzyl amine, piperazine, pyridine, pyrrazole, imidazole, pyrazine, pipyrazine, ethylenediamine, N,N′-dibenzylethylene diamine, procaine, chloroprocaine, choline, dicyclohexyl amine, and N-methylglucamine.

Non-limiting examples of suitable ammonium salts include a triethylammonium salt, a diisopropylammonium salt, an ethanolammonium salt, a diethanolammonium salt, a triethanolammonium salt, a morpholinium salt, an N-methylmorpholinium salt, a piperidinium salt, an N-methylpiperidinium salt, an N-ethylpiperidinium salt, a dibenzylammonium salt, a piperazinium salt, a pyridinium salt, a pyrrazolium salt, an imidazolium salt, a pyrazinium salt, an ethylenediammonium salt, an N,N′-dibenzylethylenediammonium salt, a procaine salt, a chloroprocaine salt, a choline salt, a dicyclohexylammonium salt, and a N-methylglucamine salt.

Non-limiting examples of hydrates include, but are not limited to, a hemihydrates, monohydrates, dihydrates, trihydrates or hexahydrates.

Non-limiting examples of solvate include alcohol solvates, such as methanol, ethanol, propanol or butanol solvates.

### Methods for Treating or Preventing Viral Infections

Provided herein are methods for treating or preventing a viral infection in a subject in need thereof, comprising administering to the subject an effective amount of an antiviral compound as described herein. In some embodiments, the viral infection is COVID-19. In some embodiments, the antiviral compound is dalcetrapib, dalcetrapib thiol or dalcetrapib thiol dimer, or a pharmaceutically acceptable salt, hydrate or solvate thereof. In some embodiments, the methods for treating or preventing a viral infection in a subject in need thereof comprise administering to the subject an effective amount of dalcetrapib, dalcetrapib thiol or dalcetrapib thiol dimer, or a pharmaceutically acceptable salt, hydrate or solvate thereof. In some embodiments, the methods for treating or preventing COVID-19 in a subject in need thereof comprise administering to the subject an effective amount of dalcetrapib, dalcetrapib thiol or dalcetrapib thiol dimer, or a pharmaceutically acceptable salt, hydrate or solvate thereof. In some embodiments, the methods for treating or preventing COVID-19 in a subject in need thereof comprise administering to the subject an effective amount of dalcetrapib or a pharmaceutically acceptable salt, hydrate or solvate thereof.

In some embodiments, the viral infection is by a virus of the family coronaviridae, picornaviridae, togoviridae, or paramyxoviridae. In some embodiments, the virus causes infant bronchiolitis, viral pneumonia, acute respiratory syndrome, meningitis, a gastrointestinal (GI) infection, common cold, summer flu, hand-foot and mouth disease, poliomyelitis, asthma, asthma exacerbation of an allergy, encephalitis, heart disease, hepatitis A, equine encephalitis, smallpox, rubella, respiratory infection, infant bronchiolis, or viral pneumonia.

In some embodiments, the virus is a coronavirus. In some embodiments, the coronavirus is an alpha, beta, gamma, or delta coronavirus. In some embodiments, the coronavirus is 229E, NL63, OC43, or HKU1. In some embodiments, the coronavirus is MERS-CoV. In some embodiments, the virus is SARS-CoV. In some embodiments, the coronavirus is SARS-CoV-2.

In some embodiments, the virus is an Enterovirus such as Coxsackievirus or Echovirus. In some embodiments, the Coxsackievirus is Enterovirus A, Enterovirus B, or Enterovirus C. In some embodiments, the Enterovirus is Enterovirus 71, DHV-1a, or DHV-3.

In some embodiments, the virus is a Poliovirus. In some embodiments, the Poliovirus is Human Poliovirus 1, Human Poliovirus 2, or Human Poliovirus 3.

In some embodiments, the virus is Rhinovirus. In some embodiments, the Rhinovirus is Human Rhinovirus A, Human a Rhinovirus B, or Human Rhinovirus C.

In some embodiments, the virus is an Aphthovirus. In some embodiments, the Aphthovirus is Bovine Rhinitis A Virus, Bovine Rhinitis B Virus, Equine Rhinitis A Virus, or Foot-and-Mouth Disease Virus.

In some embodiments, the virus is a Cardiovirus. In some embodiments, the Cardiovirus is Encephalomyocarditis Virus or Theilovirus.

In some embodiments, the virus is a Hepatovirus. In some embodiments, the Hepatovirus is Hepatovirus A, Hepatovirus B, Hepativirus C, Hepatovirus D, Hepatovirus E, Hepatovirus F, Hepatovirus G, Hepatovirus H, or Hepatovirus I.

In some embodiments, the virus is an Alphavirus. In some embodiments, the Alphavirus is Narmah Forest Virus, Eastern Equine Encephalitis Virus, Middleburg Virus, Ndumu Virus, Bebaru Virus, Chikungunya Virus, Getah Virus, Mayaro Virus, O‘nyong’nyong Virus, Ross River Virus, Semliki Forest Virus, Cabassou Virus, Everglades Virus, Mosso das Pedras Virus, Mucambo Virus, Paramana Virus, Pizuna Virus, Rio Negro Virus, Trocara Virus, Venezuelan Equine Encephalitis Virus, Aura Virus, Babanki Virus, Kyzylagach Virus, Sindbis Virus, Ockelbo Virus, Whataroa Virus, Eilat Virus, Mwinilunga Virus, Tonate Virus, or Caaingua Virus.

In some embodiments the virus is a Poxvirus. In some embodiments, the Poxvirus is Smallpox, Molliscum Contagiosum Virus, Monkeypox, or Orf Virus.

In some embodiments, the virus is a Rubivirus. In some embodiments, the Rubivirus is Rubella Virus.

In some embodiments the virus is a Parainfluenza Virus. In some embodiments, the Parainfluenza Virus is Human Parainfluenza Virus Type 1 (HPIV-1), HPIV-2, HPIV-3, or HPIV-4.

In some embodiments, the virus is Respiratory Syncytial Virus (RSV). In some embodiments, the RSV is a Type A or a Type B RSV.

In some embodiments, a method for treating or preventing COVID-19 in a subject in need thereof comprises administering to the subject an effective amount of dalcetrapib, an analog of dalcetrapib, or a pharmaceutically acceptable salt, hydrate or solvate thereof. In some embodiments, the COVID-19 is caused by SARS-CoV-2. In some embodiments, the SARS-CoV-2 is an L-strain. In some embodiments, the SARS-CoV-2 is an S-strain.

In some embodiments, the dalcetrapib, an analog of dalcetrapib, or pharmaceutically acceptable salt, hydrate or solvate thereof is administered orally. In some embodiments, the administering occurs daily. In some embodiments, the administering occurs at least 2, at least 3, at least 4, or at least 5 times per week.

In some embodiments, the effective amount is about 10 mg per day to about 1200 mg per day. In some embodiments, the effective amount is about 100 mg per day, about 200 mg per day, about 300 mg per day, about 400 mg per day, about 500 mg per day, about 600 mg per day, about 700 mg per day, about 800 mg per day, about 900 mg per day, about 1000 mg per day, about 1100 mg per day, or about 1200 mg per day. In some embodiments, the effective amount is in the range of about 1200 mg per day to about 2000 mg per day.

In some embodiments, the method comprises administering to the subject an additional antiviral agent. In some embodiments, the additional antiviral agent is Abacavir, Acyclovir, Adefovir, Amantadine, Ampligen, Amprenavir, Arbidol, Atazanavir, Atripla, Balavir, Baloxavir Marboxil, Biktarvy, Boceprevir, Cidofovir, Cobicistat, Combivir, Daclatasvir, Darunavir, Delavirdine, Descovy, Didanosine, Docosanol, Dolutegravir, Doravirine, Ecoliever, Edoxudine, Efavirenz, Elvitegravir, Emtricitabine, Enfuvirtide, Entecavir, Etravirine, Famciclovir, Fomivirsen, Fosamprenavir, Foscarnet, Fosfonet, Ganciclovir, Ibacitabine, Ibalizumab, Idoxuridine, Imiquimod, Imunovir, Indinavir, Inosine, Integrase inhibitor, Interferon type I, Interferon type II, Interferon type III, Lamivudine, Letermovir, Lopinavir, Loviride, Maraviroc, Methisazone, Moroxydine, Nelfinavir, Nevirapine, Nexavir, Nitazoxanide, Norvir, Oseltamivir, Peginterferon alfa-2a, Peginterferon alfa-2b, Penciclovir, Peramivir, Pleconaril, Podophyllotoxin, Pyramidine, Raltegravir, Remdesivir, Ribavirin, Rilpivirine, Rimantadine, Ritonavir, Saquinavir, Simeprevir, Sofosbuvir, Stavudine, Telaprevir, Telbivudine, Tenofovir alafenamide, Tenofovir disoproxil, Tenofovir, Tipranavir, Trifluridine, Trizivir, Tromantadine, Emtricitabine and Tenofovir Disoproxil Fumarate, Valaciclovir, Valganciclovir, Vicriviroc, Vidarabine, Viramidine, Zalcitabine, Zanamivir, orZidovudine. In some embodiments, the additional antiviral agent is remdesivir. In some embodiments, the method described herein do not comprise administering to the subject an additional antiviral agent.

In some embodiments, the subject of the forgoing methods is a mammal, such as a human, mouse, rat, guinea pig, dog, cat, horse, cow, pig, or non-human primate, such as a monkey, chimpanzee, or baboon. In some embodiments, the subject is a human. In some embodiments, the subject is an adult human. In some embodiments, the subject is a pediatric human.

In some embodiments, the antiviral compound inhibits (i) the replication of the virus in the subject, or (ii) the activity of a virulence factor in the subject. In some embodiments, the antiviral compound inhibits (i) the replication of SARS-CoV-2 in the subject, or (ii) the activity of a SARS-CoV-2 virulence factor in the subject. In some embodiments, the SARS-CoV-2 virulence factor is the viral envelope (E) protein, membrane (M) protein, the nucleocapsid (N) protein, the spike (S) protein, or the 3C-like protease (3CLpro).

### Methods for Inhibiting Replication of a Virus in a Cell

Also provided herein are methods for inhibiting replication of a virus in a cell, comprising contacting the cell with an effective amount of an antiviral compound provided herein. In some embodiments, the virus is MERS-CoV, SARS-CoV or SARS-CoV-2. In some embodiments, the antiviral compound is dalcetrapib, dalcetrapib thiol or dalcetrapib thiol dimer, or a pharmaceutically acceptable salt, hydrate or solvate thereof. In some embodiments, the methods for inhibiting replication of a virus in a cell comprise contacting the cell with an effective amount of dalcetrapib, dalcetrapib thiol or dalcetrapib thiol dimer, or a pharmaceutically acceptable salt, hydrate or solvate thereof. In some embodiments, the methods for inhibiting replication of SARS-CoV-2 in cell comprise contacting the cell with an effective amount of dalcetrapib, dalcetrapib thiol or dalcetrapib thiol dimer, or a pharmaceutically acceptable salt, hydrate or solvate thereof. In some embodiments, the methods for inhibiting replication of SARS-CoV-2 in cell comprise contacting the cell with an effective amount of dalcetrapib or a pharmaceutically acceptable salt, hydrate or solvate thereof.

In some embodiments, the virus is a virus of the family coronaviridae, picornaviridae, togoviridae, or paramyxoviridae.

In some embodiments, the virus is SARS-CoV-2. In some embodiments, the SARS-CoV-2 is an L-strain. In some embodiments, the SARS-CoV-2 is an S-strain.

In some embodiments, the virus is a coronavirus. In some embodiments, the coronavirus is an alpha, beta, gamma, or delta coronavirus. In some embodiments, the coronavirus is 229E, NL63, OC43, or HKU1. In some embodiments, the coronavirus is MERS-CoV. In some embodiments, the virus is SARS-CoV. In some embodiments, the coronavirus is SARS-CoV-2.

In some embodiments, the virus is an Enterovirus such as Coxsackievirus or Echovirus. In some embodiments, the Coxsackievirus is Enterovirus A, Enterovirus B, or Enterovirus C. In some embodiments, the Enterovirus is Enterovirus 71, DHV-1a, or DHV-3.

In some embodiments, the virus is a Poliovirus. In some embodiments, the Poliovirus is Human Poliovirus 1, Human Poliovirus 2, or Human Poliovirus 3.

In some embodiments, the virus is Rhinovirus. In some embodiments, the Rhinovirus is Human Rhinovirus A, Human a Rhinovirus B, or Human Rhinovirus C.

In some embodiments, the virus is an Aphthovirus. In some embodiments, the Aphthovirus is Bovine Rhinitis A Virus, Bovine Rhinitis B Virus, Equine Rhinitis A Virus, or Foot-and-Mouth Disease Virus.

In some embodiments, the virus is a Cardiovirus. In some embodiments, the Cardiovirus is Encephalomyocarditis Virus or Theilovirus.

In some embodiments, the virus is a Hepatovirus. In some embodiments, the Hepatovirus is Hepatovirus A (Hepatitis A), Hepatovirus B, Hepativirus C, Hepatovirus D, Hepatovirus E, Hepatovirus F, Hepatovirus G, Hepatovirus H, or Hepatovirus I.

In some embodiments, the virus is an Alphavirus. In some embodiments, the Alphavirus is Narmah Forest Virus, Eastern Equine Encephalitis Virus, Middleburg Virus, Ndumu Virus, Bebaru Virus, Chikungunya Virus, Getah Virus, Mayaro Virus, O‘nyong’nyong Virus, Ross River Virus, Semliki Forest Virus, Cabassou Virus, Everglades Virus, Mosso das PEdras Virus, Mucambo Virus, Paramana Virus, Pizuna Virus, Rio Negro Virus, Trocara Virus, Venezuelan Equine Encephalitis Virus, Aura Virus, Babanki Virus, Kyzylagach Virus, Sindbis Virus, Ockelbo Virus, Whataroa Virus, Eilat Virus, Mwinilunga Virus, Tonate Virus, or Caaingua Virus.

In some embodiments the virus is a Poxvirus. In some embodiments, the Poxvirus is Smallpox, Molliscum Contagiosum Virus, Monkeypox, or Orf Virus.

In some embodiments, the virus is a Rubivirus. In some embodiments, the Rubivirus is Rubella Virus.

In some embodiments the virus is a Parainfluenza Virus. In some embodiments, the Parainfluenza Virus is Human Parainfluenza Virus Type 1 (HPIV-1), HPIV-2, HPIV-3, or HPIV-4.

In some embodiments, the virus is Respiratory Syncytial Virus (RSV). In some embodiments, the RSV is a Type A or a Type B RSV.

In some embodiments, the cell is a prokaryotic cell. In some embodiments, the cell is a eukaryotic cell. In some embodiments, the cell is a mammalian cell. In some embodiments, the cell is a human cell. In some embodiments, the cell is a bacterial cell. In some embodiments, the cell is a yeast cell.

In some embodiments, the cell is in vivo. In some embodiments, the cell is ex vivo.

### Dosages and Effective Amounts

The dosage or effective amount of an antiviral compound can be selected in accordance with a variety of factors including type, species, age, weight, sex and medical condition of the subject; the severity of the disorder to be treated or prevented; the route of administration; the renal or hepatic function of the subject, the type of viral infection, the type of virus or the type of cell.

In some embodiments, the daily dosage amount of antiviral compound ranges from about 1 mg to about 1000 mg.

In some embodiments, the antiviral compound is administered to a subject at a dose of about 10 mg per day to about 1200 mg per day. For example, the effective amount may be about 100 mg per day, about 200 mg per day, about 300 mg per day, about 400 mg per day, about 500 mg per day, about 600 mg per day, about 700 mg per day, about 800 mg per day, about 900 mg per day, about 1000 mg per day, about 1100 mg per day, or about 1200 mg per day. In some embodiments, the effective amount is in the range of about 1200 mg per day to about 2000 mg per day. In some embodiments, the effective amount is in the range of about 600 mg per day to about 3900 mg per day. In some embodiments, the effective amount is greater than 3900 mg per day. In some embodiments, the antiviral compound is administered orally to a subject at an aforementioned dose.

In some embodiments, the antiviral compound is administered daily for an effective period of time. In some embodiments, the effective period of time is about 1 day to about 60 days. For example, the effective period of time may be about 1 day, about 2 days, about 3 days, about 4 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 35 days, about 40 days, about 45 days, about 50 days, about 55 days, or about 60 days. In some embodiments, the effective period of time is about 14 days. In some embodiments, the effective period of time is about 30 days. In some embodiments, the effective period of time is about 45 days. In some embodiments, the effective period of time is about 60 days.

### Compositions and Kits

Also provided herein are compositions useful for treating or preventing a viral infection in a subject or for inhibiting replication of a virus in a cell, the compositions comprising a) an effective amount of an antiviral compound; and b) a pharmaceutically acceptable carrier or vehicle.

In certain embodiments, the pharmaceutical acceptable carrier or vehicle is a liquid, such as water or an oil, including those of petroleum, animal, vegetable, or synthetic origin, such as peanut oil, soybean oil, mineral oil, sesame oil and the like. In some embodiments the compositions further comprise a pharmaceutical excipient such as saline, gum acacia, gelatin, starch paste, talc, keratin, colloidal silica, urea and the like. In addition, auxiliary, stabilizing, thickening, lubricating, and coloring agents can be used. In some embodiments, the pharmaceutically acceptable excipients are sterile when administered to a subject. Water is a useful excipient when the antiviral compound is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also comprise minor amounts of wetting or emulsifying agents, or pH buffering agents.

The compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue, or inhalation via inhalers; (2) parenteral administration, for example, by subcutaneous, intramuscular, intravenous or epidural injection as, for example, a sterile solution or suspension, or sustained release formulation; (3) topical application, for example, as a cream, ointment, or a controlled release patch or spray applied to the skin; (4) intravaginally or intrarectally, for example, as a pessary, cream or foam; (5) sublingually; (6) ocularly; (7) transdermally; (8) nasally; (9) intranasally, or (10) administration via inhalation (e.g., via an inhaler).

Compositions may include those suitable for oral, intranasal, topical (including buccal and sublingual), rectal, vaginal or parenteral administration, or those suitable for administration via inhalation (e.g., via an inhaler).

Illustrative compositions suitable for administration as a nasal spray are known in the art (See, e.g., U.S. Pat. Nos. 6,824,762; 6,565,832; 6,958,142). For example, a nasal spray composition may comprise an antiviral compound in aqueous solution, and may also comprise microcrystalline cellulose and carboxymethylcellulose sodium, dextrose, 0.02% (w/w) benzalkonium chloride, polysorbate 80, and 0.25% (w/w) phenylethyl alcohol, with a pH between about 5 and about 7.

Illustrative compositions suitable for administration via inhalation are also known in the art (See, e.g., U.S. Pub No. 2010/0143269; U.S. Pat. Nos. 9,238,031 and 9,050,267). For example, a composition for administration via inhalation may comprise micronized particles of an antiviral compound, a propellant (e.g., HVA-134a (1,1,1,2-tetrafluoroethane)), dehydrated alcohol, and oleic acid.

The compositions may conveniently be presented in unit dosage form and may be prepared by any methods well known in the art of pharmacy. The amount of antiviral compound that can be combined with a carrier material to produce a single dosage form can vary depending upon the host being treated, the particular mode of administration. The amount of antiviral compound can be combined with a carrier material to produce a single dosage form will generally be that amount of the compound which produces a therapeutic effect. Generally, out of one hundred percent, this amount will range from about 0.1 percent to about ninety-nine percent of active ingredient, e.g., from about 5 percent to about 70 percent, or from about 10 percent to about 30 percent.

In some embodiments, a composition comprises an excipient such as one or more cyclodextrins, celluloses, liposomes, micelle forming agents, e.g., bile acids, and polymeric carriers, e.g., polyesters and polyanhydrides. In certain embodiments, an aforementioned composition renders orally bioavailable the antiviral composition.

Methods of preparing these compositions can comprise admixing the antiviral compound with the carrier and, optionally, with one or more accessory ingredients. In general, the compositions are prepared by uniformly and intimately admixing the antiviral compound with liquid carriers, or finely divided solid carriers, or both, and then, if necessary, shaping the product.

Compositions suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) or as mouth washes and the like, each containing a predetermined amount of dalcetrapib as an active ingredient. An antiviral compound can also be administered as a bolus, electuary or paste.

In solid dosage forms for oral administration (capsules, tablets, pills, dragees, powders, granules, trouches and the like), the antiviral compound can be admixed with one or more pharmaceutically-acceptable carriers, such as sodium citrate or dicalcium phosphate, or one or more of the following: (1) fillers or extenders, such as starches, lactose, sucrose, glucose, mannitol, or silicic acid; (2) binders, such as, for example, carboxymethylcellulose, alginates, gelatin, polyvinyl pyrrolidone, sucrose or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules, pills and granules, may optionally be scored or prepared with coatings and shells, such as enteric coatings and other coatings well known in the pharmaceutical-formulating art. They may also be formulated so as to provide slow or controlled release of the antiviral compound therein using, for example, hydroxypropylmethyl cellulose in varying proportions to provide the desired release profile, other polymer matrices, liposomes or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions can also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of an antiviral compound can include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the antiviral compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Compositions of the pharmaceutical compositions for rectal or vaginal administration may be presented as a suppository, which may be prepared by admixing an antiviral compound with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the antiviral compound.

Compositions described herein which are suitable for vaginal administration also include pessaries, tampons, creams, gels, pastes, foams or spray compositions containing such carriers as are known in the art to be appropriate.

Dosage forms for the topical or transdermal administration of a compound can include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an antiviral compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an antivral compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of an antiviral compound to a subject. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Pharmaceutical compositions suitable for parenteral administration can comprise an antiviral compound and one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the composition isotonic with the blood of the intended recipient or suspending or thickening agents.

Examples of suitable aqueous and nonaqueous carriers which may be employed include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of an antiviral compound, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable compositions are also prepared by entrapping the drug in liposomes or microemulsions which are compatible with body tissue.

When an antiviral compound is administered as a component of a pharmaceutical composition, to humans or non-human animals, it can be administered per se or as a component of a pharmaceutical composition comprising, for example, 0.1 to 99% (more preferably, 10 to 30%) of antiviral compound by weight, in combination with a pharmaceutically acceptable carrier.

The compositions may be administered orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of an antiviral compound other than directly into the central nervous system, such that it enters the subject's system and, thus, is subject to metabolism and other like processes, for example, subcutaneous administration.

An antiviral compound can be administered to humans and other animals for therapy by any suitable route of administration, including orally, nasally, intranasally, as by, for example, a spray, rectally, intravaginally, parenterally, intraocularly, intracisternally and topically, as by powders, ointments or drops, including buccally and sublingually. In some embodiments, the antiviral compound may be administered via inhalation (e.g., via an inhaler).

Regardless of the route of administration selected, the pharmaceutical compositions described herein, are formulated into pharmaceutically-acceptable dosage forms by conventional methods known to those of skill in the art. Actual dosage levels of the active ingredients may be varied so as to obtain an amount of the active ingredient which is effective to achieve the desired therapeutic response for a particular subject, composition, and mode of administration, without being toxic to the subject.

The selected dosage level can depend upon a variety of factors including the activity of the antiviral compound, the route of administration, the time of administration, the rate of excretion or metabolism of the particular compound being employed, the rate and extent of absorption, the duration of the treatment, other drugs, compounds or materials used in combination with the antiviral compound employed, the age, sex, weight, condition, general health and prior medical history of the patient being treated, and like factors well known in the medical arts.

A physician or veterinarian can readily determine and prescribe an effective amount of the pharmaceutical composition. For example, the physician or veterinarian could start doses of an antiviral compound at levels lower than that required in order to achieve the desired therapeutic effect and gradually increase the dosage until the desired effect is achieved.

In some embodiments, a suitable daily dose of an antiviral compound is that amount that is the lowest dose effective to produce a prophylactic or therapeutic effect. Such an effective dose can depend upon the factors described above.

If desired, the effective daily dose of the antiviral compound may be administered as two, three, four, five, six or more sub-doses administered separately at appropriate intervals throughout the day, optionally, in unit dosage forms, e.g., one administration per day.

Also provided herein are kits useful for treating or preventing a viral infection, such as COVID-19. In some embodiments, the kits comprise an effective amount of an antiviral compound and instructions for its use. In some embodiments, the kits comprise an effective amount of dalcetrapib, dalcetrapib thiol or dalcetrapib thiol dimer, or a pharmaceutically acceptable salt, hydrate or solvate thereof, and instructions for use thereof.

### EXAMPLES

The following examples, which are included herein for illustration purposes only, are not intended to be limiting.

Dalcetrapib is a lipid modulator that acts by binding covalently to a cysteine residue (Cys13) in a hydrophobic pocket of the target cholesteryl ester transfer protein, and, without being bound by theory, this bond is a prerequisite for its pharmacological activity. Dalcetrapib is a lipophilic thiol ester with a logP close to 7.0. The pharmacologically active free thiol form of dalcetrapib is generated by non-specific esterases and lipases present in body fluids such as plasma, cell homogenates, intra-cellular milieu or at basic pH. Dalcetrapib thiol also binds to a cysteine residue (Cys133) in a hydrophobic pocket of MD2, a regulator of the toll-like receptor 4 receptor signalling pathway.

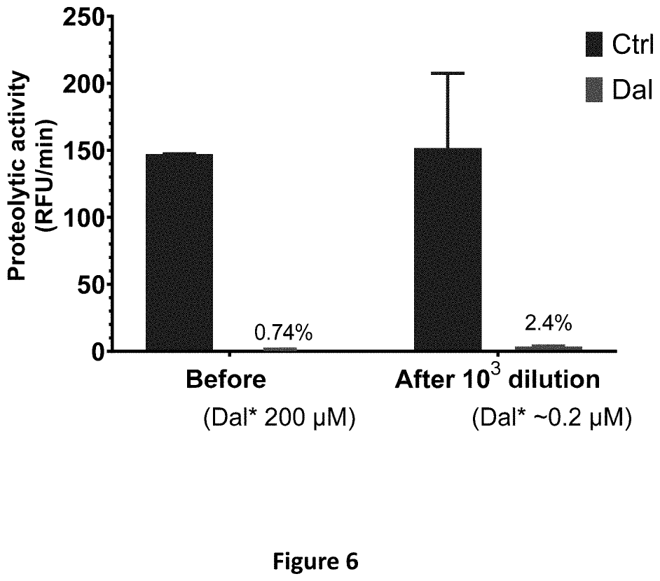

As described herein, the ability of dalcetrapib to inhibit SARS-CoV-2 3CL protease activity and SARS-CoV-2 proliferation was demonstrated. Docking investigations demonstrated that dalcetrapib thiol binds to the catalytic site of the 3CL protease with a delta G value of −8.4 Kcal/mol. Dalcetrapib thiol inhibited both 3CL protease activity in vitro and viral replication in Vero 6 cells with IC₅₀S of 14.4±3.6 μM (FIG. 7) and about 17.8 μM (FIG. 6), respectively. Near-complete inhibition of protease activity persisted despite elution of dalcetrapib, suggesting stable protease-drug interaction. The inhibitory effect of dalcetrapib on SARS-CoV-2's main 3CL protease and viral replication warrants its clinical evaluation for the treatment of COVID-19.

### Example 1: Testing the Anti-Viral Activity of an Antiviral Compound In Vitro

The ability of an antiviral compound, such as dalcetrapib, dalcetrapib thiol or dalcetrapib thiol dimer, or a pharmaceutically acceptable salt, hydrate or solvate thereof, to inhibit one or more SARS-CoV viruses is investigated in vitro. The human coronavirus OC43 (HCoV-OC43), a member of the species Betacoronavirus, is used in this assay. HCoV-OC43 is an enveloped, positive-sense, single-stranded RNA virus that infects, for example, humans, non-human primates, and cattle. The antiviral compound is tested at concentrations from 0.1 to 100 μM.

Vero E6 cells, kidney cells from African Green Monkey (Sigma catalog no. 85020205), support the growth of slowly-replicating viruses. Cells are seeded in flat bottom, 96-well plates at a density of approximately 9×10³ cells per well. The cells are then either (i) mock-infected with serum-free DMEM or (ii) infected with SARS-CoV OC43 (at a dose of 100-fold the median tissue culture infective dose of per well) in 100 μL of serum-free MEM and incubated for 1 hour at 37° C. in a 5% CO2 atmosphere. The viral inoculum is removed after the 1 hour incubation period, and then 100 μL of MEM, supplemented with 2% FCS and the inhibitors to be tested is added, with inhibitor concentrations ranging from 0.1 to 100 μM. Cells are then incubated for an additional 48 hour at 37° C. in a 5% CO2 atmosphere. All controls and each inhibitor concentration are set up in triplicate, and the antiviral assays are performed independently on at least two separate occasions. Viral RNA in inoculum and infected cells is quantitated by PCR.

Cell viability is determined approximately 48 hours after infection using the CellTiter-Glo luminescent cell viability assay (Promega).

### Example 2: In Silico Analysis of Docking of an Antiviral Compound to Viral or Host Proteins

An in silico analysis was used to examine docking of an antiviral compound, such as dalcetrapib, dalcetrapib thiol or dalcetrapib thiol dimer, or pharmaceutically acceptable salt, hydrate or solvate thereof, to (i) virus proteins and enzymes such as the spike (S) protein, the envelope (E) protein, the membrane (M) protein, the nucleocapsid (N) protein, and the 3C-like protease (3CLpro), and (ii) host cell proteins such as the angiotensin-converting enzyme 2 (ACE2) receptor, which binds to the viral spike (S) protein, and the host serine protease TMPRSS2 and DPP4, which are involved in spike protein cleavage. An assessment of docking was performed by calculating binding free energy (ΔG) using AutoDock Vina5 or Swissdock (Grosdidier et. al., Proteins. 2007 Jun. 1; 67(4):1010-25. and Grosdidier et al Nucleic Acids Res. 2011 Jul. 1; 39).

In one experiment, AG values were determined for coronavirus protease structures obtained from the Protein Data Bank (PDB). PDB Accession No. ID 6LU7 (SARS-CoV-2 3CLpro) had AG values from −6.1 to −7.45 Kcal/mol. PDB Accession No. 2DUC (SARS-CoV2 3CLpro) had ΔG values of −6.8 Kcal/mol for the A chain and −8.43 Kcal/mol for the dimer A and B chain.

### Example 3: Testing Antiviral Compound in a MucilAir™ Model

MucilAir™ is a model of a 3-dimensional human airway epithelia, which is reconstituted with epithelial cells and used for testing in vitro. At T=0, 100 μL of viral inoculum (i.e., SARS-CoV-2) is applied to the apical side of the MucilAir™ pool for 1 h at 37° C. Epithelia are then washed with PBS (with Ca²⁺/Mg²⁺) in order to remove the inoculum.

Apical washes are performed with 200 μL of MucilAir™ culture media for 10 min at 37° C. for 1, 24 and 48-hour time points. 10 μl of antiviral compound (e.g., dalcetrapib, dalcetrapib thiol or dalcetrapib thiol dimer, or a pharmaceutically acceptable salt, hydrate or solvate thereof) in ethanol or DMSO is renewed at 1, 24 and 48 hours. Final concentration of antiviral compound is in the range of about 0.1 to about 100 μM.

Cellular supernatant is lysed and viral RNA extracted with the QIAamp Viral RNA Mini Kit (Qiagen ref 52906). Viral RNA is quantified by RT-qPCR (Taqman™ one step) with the Quantstudio5 (Applied Thermofisher). Data are analyzed by DCt calculation and presented as fold change of genome copy number (ORF1b-nsp14 gene of SRAS-CoV-2). Samples collected from apical washes at different time-points are separated into 2 tubes: one for TCID50 viral titration and one for RT-qPCR. RT-qPCR analysis is performed for all conditions, and TCID50 is performed only for selected compounds and time-points found by RT-qPCR analysis.

### Example 4: Inhibition of the SARS-CoV-2 Main 3CL Protease and SARS-CoV-2 Replication by Dalcetrapib Thiol

This Example demonstrates the ability of dalcetrapib thiol to inhibit SARS-CoV-2 3CL protease activity and SARS-CoV-2 proliferation.

### Docking of Dalcetrapib Thiol

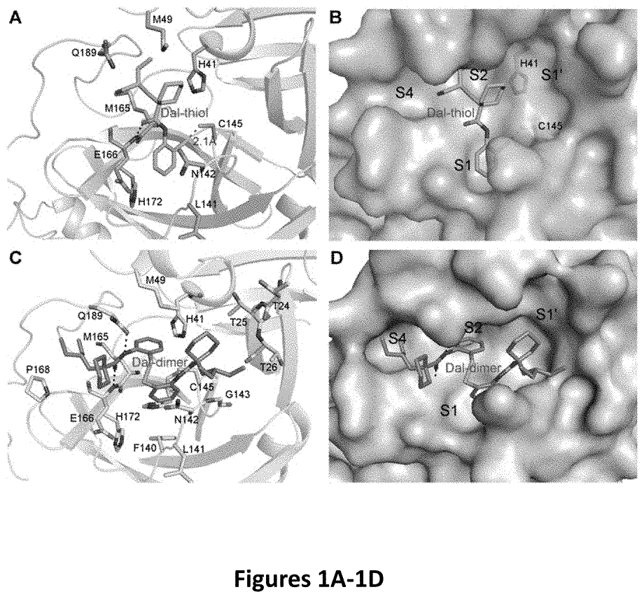

Molecular docking was performed using LeDock software to evaluate the potential binding modes of dalcetrapib thiol to the main 3CL protease of SARS-CoV-2. The best ranking poses of dalcetrapib thiol in both standard and SCAR protocols revealed very similar binding of this compound in the active site of the main protease (FIG. 1A). In both cases, dalcetrapib thiol binds to the active site of the main protease with its carbonyl group anchored through a hydrogen bond by the main chain amide group of Glu166. Meanwhile, dalcetrapib thiol's aromatic ring is inserted in the S1 site, which recognizes the Gln residue (P1 residue) of substrate proteins, and sandwiched between Glu166 and Asn142, whereas its 2-ethylbutyl and cyclohexane moieties are snugly bound in the S2 and S4 sites. Without being bound by theory, the van der Waals contacts with Met49, Met165, His41 and Gln189 appear to be crucial for the stabilization of dalcetrapib thiol. The sulfur atom of dalcetrapib thiol is 3.7 Å away from the Cys145 thiol of the protease in the standard protocol, whereas this distance is shortened to 2.1 Å in the SCAR protocol (FIGS. 1A and 1B). This indicates the feasibility of disulfide bond formation between dalcetrapib thiol and Cys145 of the main 3CL protease, and the binding pose of dalcetrapib thiol obtained in the standard protocol could be in the trajectory towards the disulfide bond formation. The creation of a covalent bond could be facilitated by slight rotations or changes of both the aromatic ring of dalcetrapib thiol and the thiol group of Cys145 of the main protease in order to achieve appropriate bond angles. Several ligands were bound to the active site of the main protease with their aromatic rings bound to the S1 site (e.g., PDB 5R84), as observed in our docking trials. On the other hand, the docking trials of dalcetrapib thiol dimer indicated that its larger size could utilize all the S1′, S1, S2 and S4 sites to achieve its fitting in the active site pocket, which is somewhat similar to that observed for the N3 inhibitor (FIGS. 1C and 1D). Despite the presence of four polar atoms in dalcetrapib thiol dimer, the major driving forces are likely contributed by numerous hydrophobic interactions since only two H-bonds are established between this dimer molecule and the main protease (FIG. 1C). These H-bonds occur in only one dalcetrapib moiety of dalcetrapib thiol dimer, with its carbonyl group hydrogen binding to the main chain amide of Glu166 and its amide group being stabilized by the side chain carbonyl of Gln189.

### Inhibition of Enzymatic Activity of the Main 3CL Protease by Dalcetrapib Thiol

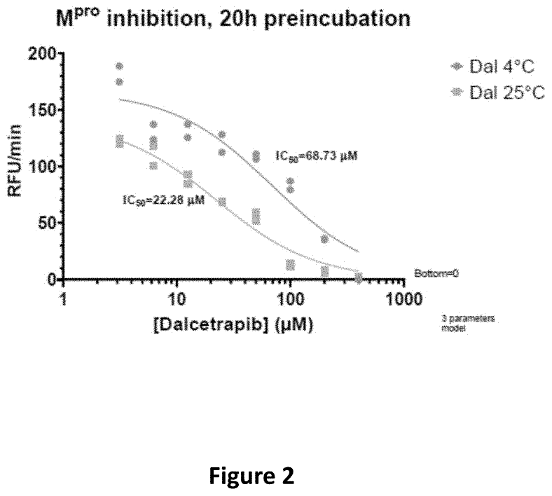

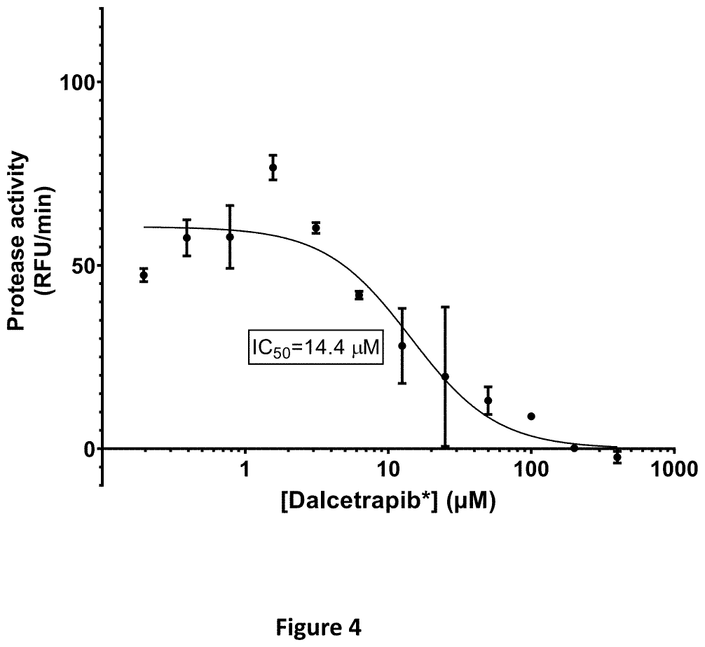

The ability of dalcetrapib thiol to inhibit the main 3CL protease of SARS-CoV-2 in vitro was also evaluated (FIGS. 2 and 4). When dalcetrapib thiol was incubated for 20 hours in presence of the main 3CL protease at room temperature before addition of the substrate for the 3CL protease, the IC₅₀ was 14.4±3.6 μM. Shorter incubation of dalcetrapib thiol for one hour with the main protease resulted in a much higher IC₅₀ (100±3.6 μM).

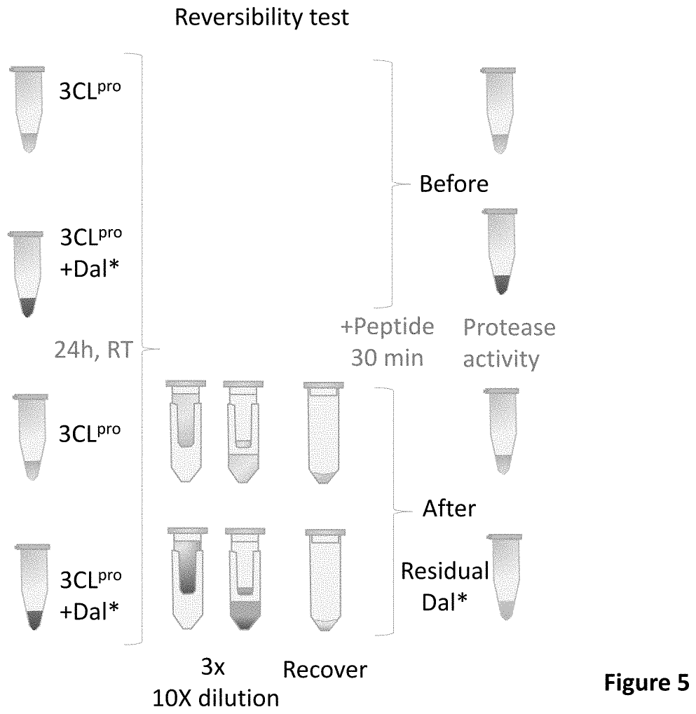

The reversibility of the inhibitory activity of dalcetrapib thiol was then evaluated according to the protocol shown in FIG. 5. Preactivated dalcetrapib in its thiol form was incubated for 24 hours with the main 3CL protease and the solution containing both was then submitted to three cycles of dilution and filtration through a 3 kDa porous membrane, thus allowing elution of free dalcetrapib thiol. Once the main 3CL protease was exposed to a high concentration of dalcetrapib, near-complete inhibition (97.6%) of protease activity persisted despite elution of dalcetrapib which brought its concentration down to 0.2 μM, suggesting the presence of stable protease-drug interaction (FIG. 6). Exposure to 10 mM of DTT (dithiothreitol) or TCEP (tris(2-carboxyethyl)phosphine) at that stage did not result in recovery of the protease activity (data not shown).

### Inhibition of SARS-CoV-2 Proliferation by Dalcetrapib

Vero E6 cells were grown in a 24-well plate to 80-100% confluence. On the day of infection, a viral suspension comprising SARS-CoV-2 in MEM (minimum essential medium)+2% FBS (fetal bovine serum) was prepared. An aliquot of 0.2 ml of viral suspension was added to each well, and plates were incubated at 37° C. for 90 minutes to allow for viral adsorption. Viral titer was selected to produce about 40 plaques per well (i.e., in wells that were not treated with any drug).

Subsequently, the medium was removed and an overlay consisting of agarose and an equal volume of antiviral compound, such as dalcetrapib, dalcetrapib thiol or dalcetrapib thiol dimer, or a pharmaceutically acceptable salt, hydrate or solvate thereof, in MEM+2% FVS (final concentration) was added. The antiviral compound was tested at a concentration in the range from about 0.1 to 100 μM (final diluted concentration). Each antiviral compound concentration was tested in triplicate, plus negative control (no drug). The plate was incubated at 37° C. for 3-4 days.

After the incubation period, the cells were fixed with 0.5 ml of 3.7% formaldehyde for 30-60 minutes. The agarose was then removed, and the cells were stained with crystal violet (0.8% in 50% ethanol). The number of viral plaques in each well was determined using an inverted microscope. The IC₅₀ value, which corresponds to the concentration of antiviral compound which reduces the number of plaques by 50% compared to no-drug wells, was then determined.

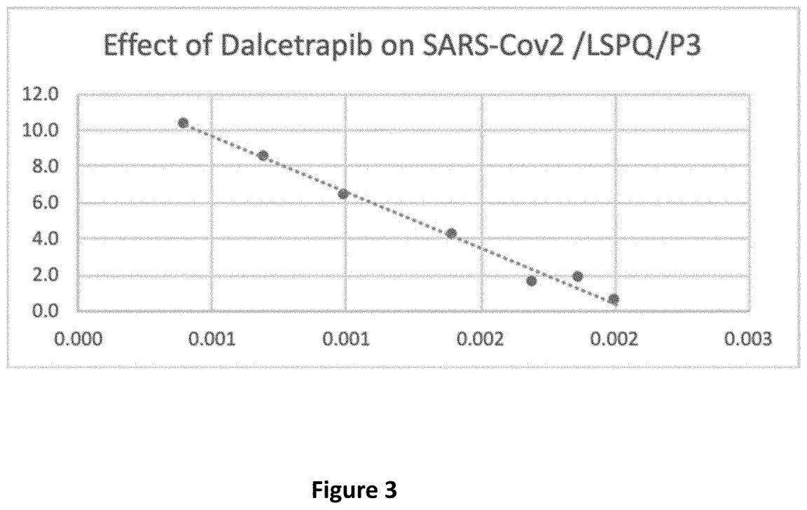

SARS-CoV-2 plaque formation was linearly reduced when Vero E6 cells were pre-incubated with increasing concentrations of dalcetrapib for one hour before the addition of the SARS-CoV-2 coronavirus resulting in an IC₅₀ of 17.8 μM (FIG. 3).

Dalcetrapib in its thiol form was shown in the current study to bind to the catalytic site of the main 3CL protease with a delta G value of −8.4 Kcal/mol, and inhibit both its activity in vitro and SARS-CoV-2 replication with IC₅₀s of 14.4±3.6 μM and about 17.8 μM, respectively. Persistence of near-complete inhibition of protease activity despite elution of dalcetrapib suggests stable protease-drug interaction.

Dalcetrapib thiol binds in the active site of the SARS-CoV-2 main 3CL protease with its carbonyl group anchored to the amide group of Glu166. The latter amino acid is necessary for substrate recognition, protease dimerization and altering proteolytic activity. The van der Waals contacts with Met49, Met165, His41 and GIn189 appear to be crucial for the stabilization of interaction with dalcetrapib thiol. The feasibility of a disulfide bond formation between dalcetrapib thiol and Cys145 of the main 3CL protease is supported by the distance between their sulfur atoms (3.7 Å). This distance was shortened to 2.1 Å in the SCAR protocol. The docking of dalcetrapib thiol dimer indicated that this dimer molecule with its larger size could utilize all the S1′, S1, S2 and S4 sites to achieve its fitting in the active site pocket, which is somewhat similar to the situation observed with the N3 inhibitor. Despite the presence of four polar atoms in the dalcetrapib thiol dimer molecule, the major driving forces are nevertheless likely hydrophobic interactions.

Dalcetrapib inhibited the enzymatic activity of the main 3CL protease with an IC₅₀ of 14.4 μM when they were pre-incubated for 20 hours (FIGS. 2 and 4). The IC₅₀ increased to 100 μM when pre-incubation time was shortened to one hour. A similar behaviour of dalcetrapib was previously observed in the inhibition of cholesteryl ester transfer protein, with the IC₅₀ decreasing from 1178 nM to 45 nM with prolongation of the pre-incubation time from 1 to 24 hours. This time-dependent inhibition of activity in vitro may reflect the time necessary for de-esterification of dalcetrapib and formation of stable interaction with the target protein. The persistence of almost complete inhibition of enzymatic activity once the main 3CL protease had been previously exposed to a high concentration of dalcetrapib despite reduction of the drug concentration down to 0.2 μM suggests stable protease-drug interaction.

Dalcetrapib decreased plaque formation by SARS-CoV-2 grown on Vero E6 cells linearly with increasing drug concentrations, with an IC₅₀ of 17.8 μM. Inhibition of the main 3CL protease and SARS-CoV-2 replication thus follows the interaction of dalcetrapib with this rate-limiting enzyme. The general concern that the formation of covalent protein adducts may result in potential safety issues has not been observed in extensive and large chronic animal and human safety studies of dalcetrapib. This may be explained by the relatively high lipophilicity of dalcetrapib, which preferentially occupies the lipophilic pocket within the protein and not exposed at the surface.

Radioactive dalcetrapib administered orally to rodents distributes in all tissues including the lungs and gastrointestinal tract (unpublished data). In rats, the percentages of an administered oral dose excreted into the feces as dalcetrapib-ester and dalcetrapib thiol plus dalcetrapib thiol dimer are 41.1% and 17.8%, respectively. The corresponding values following oral administration in monkeys are 11.6% and 22.2%. These results suggest that the intestine is exposed to high concentrations of the active compound, which may contribute to potential antiviral activity of dalcetrapib at the gastrointestinal level.

### Example 5: Treating or Preventing a SARS—Co-V2 Infection in a Subject in Need Thereof

A subject diagnosed with SARS-CoV-2 infection is treated with an effective amount of an antiviral compound, such as dalcetrapib, dalcetrapib thiol or dalcetrapib thiol dimer, or a pharmaceutically acceptable salt, hydrate or solvate thereof. The antiviral compound is administered orally, once per day, for approximately 30 days. The subject is tested periodically to measure viral titers.

If the subject's symptoms do not improve after 30 days, the treatment period may be extended. Optionally, an additional antiviral agent may also be administered to the subject, in addition to the antiviral compound.

The foregoing is illustrative of the present invention, and is not to be construed as limiting thereof. The invention is defined by the following claims, with equivalents of the claims to be included therein.

What is claimed is:

1. A method for treating or preventing a viral infection, the method comprising administering to a subject in need thereof an effective amount of dalcetrapib, dalcetrapib thiol or dalcetrapib thiol dimer, or a pharmaceutically acceptable salt, hydrate or solvate thereof.

2. The method of claim 1, wherein the viral infection is COVID-19.

3. The method of claim 2, wherein the COVID-19 is caused by SARS-CoV-2.

4. The method of claim 3, wherein the SARS-CoV-2 is an L-strain.

5. The method of claim 3, wherein the SARS-CoV-2 is an S-strain.

6. The method of claim 1, wherein the viral infection is caused by SARS-CoV, SARS-CoV-2, Enterovirus, Coxsackievirus, Echovirus, Poliovirus, Rhinovirus, Aphthovirus, Cardiovirus, Hepatovirus, Alphavirus, Poxyvirus, Rubivirus, Parainfluenza, or Respiratory Syncytial virus (RSV).

7. The method of claim 1, wherein the administering is orally administering, intranasally administering, or administering via inhalation.

8. The method of claim 1, wherein the administering occurs daily.

9. The method of claim 1, wherein the effective amount is about 10 mg per day to about 1200 mg per day.

10. The method of claim 1, wherein the effective amount is about 100 mg per day, about 200 mg per day, about 300 mg per day, about 400 mg per day, about 500 mg per day, about 600 mg per day, about 700 mg per day, about 800 mg per day, about 900 mg per day, about 1000 mg per day, about 1100 mg per day, or about 1200 mg per day.

11. The method of claim 1, wherein the administering occurs at least 2 times per week.

12. The method of claim 1, further comprising administering to the subject an additional antiviral agent.

13. The method of claim 1, wherein the method does not comprise administering to the subject an additional antiviral agent.

14. The method of claim 1, wherein the subject is a mammal.

15. The method of claim 1, wherein the subject is a human.

16. The method of claim 1, wherein the dalcetrapib, dalcetrapib thiol or dalcetrapib thiol dimer, or pharmaceutically acceptable salt, hydrate or solvate thereof, inhibits (i) the replication of the virus in the subject, or (ii) the activity of a virulence factor in the subject.

17. A method for reducing risk of acquiring a viral infection, the method comprising administering to a subject in need thereof an effective amount of dalcetrapib, dalcetrapib thiol or dalcetrapib thiol dimer, or a pharmaceutically acceptable salt, hydrate or solvate thereof.

18. The method of claim 17, wherein the viral infection is COVID-19.

19. The method of claim 18, wherein the COVID-19 is caused by SARS-CoV-2.

20. The method of claim 19, wherein the SARS-CoV-2 is an L-strain.

21. The method of claim 19, wherein the SARS-CoV-2 is an S-strain.

22. The method of claim 17, wherein the viral infection is caused by SARS-CoV, SARS-CoV-2, Enterovirus, Coxsackievirus, Echovirus, Poliovirus, Rhinovirus, Aphthovirus, Cardiovirus, Hepatovirus, Alphavirus, Poxyvirus, Rubivirus, Parainfluenza, or Respiratory Syncytial virus (RSV).

23. The method of claim 17, wherein the administering is orally administering, intranasally administering, or administering via inhalation.

24. The method of claim 17, wherein the administering occurs daily.

25. The method of claim 17, wherein the administering occurs at least 2 times per week.

26. The method of claim 17, wherein the subject is a mammal.

27. The method of claim 17, wherein the subject is a human.
